(19) United States
(12) Patent Application Publication
(10) Pub. No.: US 2024/0207144 A1
(43) Pub. Date: Jun. 27, 2024

(54) OIL-IN-WATER DESPERSIONS OF HYDROPHOBIC AGENTS AND POLYMER OR COPOLYMER FRAGMENTS, OR COMBINATIONS THEREOF, AND METHODS OF USE THEREOF

(71) Applicant: LEADING EDGE INNOVATIONS, LLC, Branchburg, NJ (US)

(72) Inventors: James Michael Wilmott, Banger, PA (US); Michael Ross, Easton, PA (US); Tamara Babenko, Bridgewater, NJ (US); Purvesh Patel, Piscataway, NJ (US)

(73) Assignee: LEADING EDGE INNOVATIONS, LLC, Branchburg, NJ (US)

(21) Appl. No.: 18/542,085

(22) Filed: Dec. 15, 2023

Related U.S. Application Data
(60) Provisional application No. 63/433,656, filed on Dec. 19, 2022.

(51) Int. Cl. A61K 8/04; A61K 8/81; A61K 9/10; A61Q 19/00

(52) U.S. Cl. CPC A61K 8/044; A61K 8/8147; A61K 9/10; A61Q 19/00; A61K 2800/10

(57) ABSTRACT

Compositions having one or more oil-in-water dispersions of (i) particles of one or more hydrophobic agent(s), (ii) one or more polymer or copolymer fragments, or combinations thereof, and (iii) an aqueous-solute fluid. The particles of one or more hydrophobic agent(s) are present in an amount from about 0.01% wt. to about 70% wt., the one or more polymer or copolymer fragments, or combinations thereof are present in an amount from about 0.01% wt. to about 10% wt., and the aqueous-solute fluid is present in an amount from about 1.0% wt. to about 98.5% wt., all based on the total weight of the composition. The one or more polymer or copolymer fragments, or combinations thereof, are sufficient to stabilize the particles of one or more hydrophobic agent(s) in the dispersion, at a level from about 0.05% w/w to about 70% w/w of the one or more hydrophobic agent(s), in the dispersion. Processes of making the dispersions, and method of use of the dispersions. The dispersions are useful, for example, in nutritional, pharmaceutical, biomedical, cosmetic, food, animal care, veterinary care, household, pet care, and other applications.

### CORRESPONDING APPLICATION

This application claims priority to U.S. Provisional Patent Application No. 63/433,656, filed on Dec. 19, 2022, which is incorporated herein in its entirety by reference thereto.

### BACKGROUND OF THE DISCLOSURE

### 1. Field of the Disclosure

This disclosure generally relates to oil-in-water dispersions of hydrophobic agent(s), in particular, to dispersions of (i) particles of one or more hydrophobic agent(s) having an average particle size of about 5 m or less, (ii) one or more polymer or copolymer fragments, or combinations thereof, and (iii) an aqueous-solute fluid. The dispersions are useful in nutritional, pharmaceutical, biomedical, cosmetic, food, animal care, veterinary care, household, pet care, and other applications.

### 2. Discussion of the Background Art

The current practices for combining a hydrophobic material (such as liquid, semi-solid, or solid) with a hydrophilic liquid requires the addition of agents that change the native properties of both the hydrophobic material and the hydrophilic liquids so that they more closely resemble one another. As the properties of the two phases converge because of the additives, they have a greater propensity to be stable for a commercially viable period of time. An important class of additives that can be used in these hydrophobic phase/hydrophilic phase combinations is the surface-active agent, which is typically referred to as a “surfactant”. These surfactants have both hydrophobic and hydrophilic properties.

When one or more of these agents are incorporated into the hydrophobic phase, the hydrophilic phase, or both the agents will align themselves at the hydrophobic phase-hydrophilic phase interface or at the interface between the composition and the surrounding air. The force that exists at the hydrophobic phase-hydrophilic phase (“Interfacial Tension”) is reduced, allowing the two phases to more favorably coexist. Similarly, the force that exists at the air-composition interface (“surface tension”) is also reduced.

A special sub-category of “surfactants” is called an emulsifier. When carefully selected, such emulsifiers have a wide range of surface-active properties. These materials not only lower the interfacial tension at the hydrophobic phase-hydrophilic phase interface but, with the input of shearing energy, they enable the formation of stable droplets of one phase within the other. The resulting product is called an emulsion. In many cases such emulsions are prepared by heating the hydrophobic and hydrophilic phases to a temperature of 70° C. or greater before combining the two phases. The purpose of heating the phases is to ensure that all semi-solid and solid hydrophobic materials used are melted, and that the two phases have a low enough viscosity so the two phases can mix freely. The hydrophobic and hydrophilic phases are typically mixed until they achieve a homogeneous appearance. Thereafter, they are cooled to ensure the formation of appropriately sized droplets, which is usually on average in the 3 micron to 10 micron range. Such emulsions typically have a homogeneous, opaque, white appearance due to their particle size.

These emulsions present difficulties in that the stability of these emulsions is particularly problematic when the hydrophilic phase contains one or more water-miscible solvents. Also, the processing that creates stable emulsions is difficult to scale from the laboratory to production, and they are not amenable to maintaining emulsion stability upon dilution.

### SUMMARY OF THE DISCLOSURE

It has been found that compositions containing dispersions of (i) hydrophobic agents having an average particle size of about 5 m or less, (ii) polymer or copolymer fragments, or combinations thereof, and (iii) an aqueous-solute fluid, are stable for a commercially viable period of time. The dispersions of hydrophobic agents can greatly enhance the aesthetic and therapeutic properties of the composition. Further, the dispersions of hydrophobic agents can be easily diluted in the composition post-production to deliver the preset or desired level of therapeutic agents and the preset or desired aesthetic properties. The compositions can easily be scaled from the laboratory to production.

This disclosure relates in part to a composition comprising: a dispersion comprising (i) particles of one or more hydrophobic agent(s) having an average particle size of about 5 m or less, (ii) one or more polymer or copolymer fragments, or combinations thereof, and (iii) an aqueous-solute fluid. The one or more polymer or copolymer fragments, or combinations thereof, are present in an amount sufficient to stabilize the particles of one or more hydrophobic agent(s) in said dispersion.

This disclosure also relates in part to a composition comprising a dispersion. The dispersion comprises (i) particles of one or more hydrophobic agent(s), (ii) one or more polymer or copolymer fragments, or combinations thereof, and (iii) an aqueous-solute fluid. The particles of one or more hydrophobic agent(s) are present in an amount from about 0.01% wt. to about 70% wt., the aqueous-solute fluid is present in an amount from about 1.0% wt. to about 98.5% wt., and the one or more polymer or copolymer fragments, or combinations thereof are present in an amount from about 0.01% wt. to about 10% wt., all based on the total weight of the composition. The one or more polymer or copolymer fragments, or combinations thereof, are sufficient to stabilize the particles of one or more hydrophobic agent(s) in said dispersion, at a level from about 0.05% w/w to about 70% w/w of said one or more hydrophobic agent(s), in said dispersion.

This disclosure further relates in part to a process comprising: preparing a premix comprising (i) one or more hydrophobic agent(s), (ii) one or more polymers or copolymers, or combinations thereof, (iii) an aqueous-solute fluid, and optionally (iv) one or more additive(s); subjecting the premix to low energy mixing to form a first dispersion; and subjecting the first dispersion to ultra-high energy mixing to form a second dispersion. The second dispersion comprises (i) particles of one or more hydrophobic agent(s) having an average particle size of about 5 m or less, (ii) one or more polymer or copolymer fragments, or combinations thereof, and (iii) an aqueous-solute fluid. The one or more polymer or copolymer fragments, or combinations thereof, are present in an amount sufficient to stabilize the particles of one or more hydrophobic agent(s) in said second dispersion.

This disclosure yet further relates in part to a composition comprising: a dispersion comprising (i) particles of one or more hydrophobic agent(s) having an average particle size of about 5 m or less, (ii) one or more polymer or copolymer fragments, or combinations thereof, and (iii) an aqueous-solute fluid. The one or more polymer or copolymer fragments, or combinations thereof, are present in an amount sufficient to stabilize the particles of one or more hydrophobic agent(s) in said dispersion. The composition produced by a process comprising: preparing a premix comprising (i) one or more hydrophobic agent(s), (ii) one or more polymers or copolymers, or combinations thereof, (iii) an aqueous-solute fluid, and optionally (iv) one or more additive(s); subjecting the premix to low energy mixing to form a first dispersion; and subjecting the first dispersion to ultra-high energy mixing to form a second dispersion. The composition comprises the second dispersion.

The composition comprises the second dispersion.

This disclosure further relates in part to a method of treating disorders of human or animal skin, hair or mucosal tissue. The method comprises applying to the skin, hair or external mucosa of a human or animal a composition. The one or more hydrophobic agent(s) comprise one or more therapeutic agent(s). The one or more polymer or copolymer fragments, or combinations thereof, are present in an amount sufficient to stabilize the particles of one or more hydrophobic agent(s) in said dispersion.

This disclosure yet further relates in part to a method of imparting a desirable tactile, olfactory, or visual property to a skin, hair, or mucosal surface of a human or animal, or to a surface or substrate. The method comprises applying to the skin, hair or external mucosa of a human or animal, or to a surface or substrate, a composition. The composition comprises: a dispersion comprising (i) particles of one or more hydrophobic agent(s) having an average particle size of about 5 m or less, (ii) one or more polymer or copolymer fragments, or combinations thereof, and (iii) an aqueous-solute fluid. The one or more hydrophobic agent(s) comprise one or more aesthetic modifying agent(s). The one or more polymer or copolymer fragments, or combinations thereof, are present in an amount sufficient to stabilize the particles of one or more hydrophobic agent(s) in the dispersion.

This disclosure also relates in part to a method of delivering one or more active or therapeutic ingredients to a human or animal. The method comprises providing a dispersion comprising (i) particles of one or more hydrophobic agent(s) having an average particle size of about 5 m or less, (ii) one or more polymer or copolymer fragments, or combinations thereof, and (iii) an aqueous-solute fluid. The one or more polymer or copolymer fragments, or combinations thereof, are present in an amount sufficient to stabilize the particles of one or more hydrophobic agent(s) in said dispersion. The dispersion acts as a multifunctional delivery vehicle for active or therapeutic ingredients. The method further comprises using the multifunctional delivery vehicle to deliver the one or more active or therapeutic ingredients to a human or animal.

The one or more active or therapeutic ingredients include, for example, anti-acne agents, antimicrobial agents, anti-inflammatory agents, analgesics, anti-erythemal agents, antipruritic agents, antiedemal agents, anti-psoriatic agents, antifungal agents, skin protectants, sunscreen agents, vitamins, antioxidants, anti-irritants, anti-bacterial agents, antiviral agents, antiaging agents, photoprotection agents, exfoliating agents, wound healing agents, sebum modulators, immunomodulators, hormones, botanicals, moisturizing agents, hand sanitizing agents, astringents, sensates, antibiotics, anesthetics, steroids, tissue healing substances, tissue regenerating substances, amino acids, peptides, minerals, ceramides, hyaluronic acids, skin bleaching ingredients, pre-biotics, probiotics, hemp oils, cannabinoids, and any derivatives or combinations thereof.

This disclosure further relates in part to a method for reducing transient flora on skin and improving the condition of the skin. The method comprises applying to the skin of a human or animal a composition. The composition comprises: a dispersion comprising (i) particles of one or more hydrophobic agent(s) having an average particle size of about 5 m or less, (ii) one or more polymer or copolymer fragments, or combinations thereof, and (iii) an aqueous-solute fluid. The one or more hydrophobic agent(s) comprise one or more therapeutic agent(s) and/or one or more aesthetic modifying agent(s). The one or more polymer or copolymer fragments, or combinations thereof, are present in an amount sufficient to stabilize the particles of one or more hydrophobic agent(s) in said dispersion.

In the method for reducing transient flora on skin and improving the condition of the skin, the compositions of this disclosure counteract the negative effects of high alcohol concentration on skin and help restore balance in the skin barrier. For example, the compositions of this disclosure supply the skin with moisturization and replenishing natural fatty acids that are stripped away during the use of a high alcohol product.

This disclosure yet further relates in part to a method of using a composition to enhance a physical, chemical, nutritional and/or sensory property of a food. The method comprises applying an edible composition into or onto the food. The edible composition comprises a dispersion comprising (i) particles of one or more edible hydrophobic agent(s) having an average particle size of about 5 m or less, (ii) one or more edible polymer or copolymer fragments, or combinations thereof, and (iii) an aqueous-solute fluid. The one or more edible hydrophobic agent(s) comprise one or more edible therapeutic agent(s) and/or one or more edible aesthetic modifying agent(s). The one or more edible polymer or copolymer fragments, or combinations thereof, are present in an amount sufficient to stabilize the particles of one or more edible hydrophobic agent(s) in said dispersion. The particles of the one or more edible hydrophobic agent(s) in the dispersion increase an extent of penetration of the dispersion throughout a water phase of a substrate of the food, thereby producing a bloom effect distributing the particles of the one or more edible hydrophobic agent(s) uniformly throughout the water phase of the substrate that enhances a physical, chemical, nutritional and/or sensory property of the food.

This disclosure also relates in part to a method of using a composition to enhance a physical, chemical, nutritional and/or sensory property of a beverage. The method comprises applying an edible composition into the beverage. The edible composition comprises a dispersion comprising (i) particles of one or more edible hydrophobic agent(s) having an average particle size of about 5 m or less, (ii) one or more edible polymer or copolymer fragments, or combinations thereof, and (iii) an aqueous-solute fluid. The one or more edible hydrophobic agent(s) comprise one or more edible therapeutic agent(s) and/or one or more edible aesthetic modifying agent(s). The one or more edible polymer or copolymer fragments, or combinations thereof, are present in an amount sufficient to stabilize the particles of one or more edible hydrophobic agent(s) in said dispersion. The particles of the one or more edible hydrophobic agent(s) in the dispersion increase an extent of penetration of the dispersion throughout a water phase of the beverage, thereby producing a bloom effect distributing the particles of the one or more edible hydrophobic agent(s) uniformly throughout the water phase of the beverage that enhances a physical, chemical, nutritional and/or sensory property of the beverage.

The small average particle size of about 5 m or less, monodispersity, and force of repulsion of the particles of the hydrophobic agent in the dispersion increases the extent of penetration and accelerates diffusion throughout the water phase of a substrate of the food or beverage, producing a bloom effect that enhances the physical, chemical, nutritional and/or sensory property of a food or beverage.

The dispersions of this disclosure can be applied into or onto a food and/or a beverage to enhance the physical, chemical, nutritional, and/or sensory properties of the food or beverage, and also to prevent freezer burn.

This disclosure further relates in part to a method of enhancing food. The method comprises contacting the food with a composition. The composition comprises a dispersion comprising (i) particles of one or more edible hydrophobic agent(s) having an average particle size of about 5 m or less, (ii) one or more edible polymer or copolymer fragments, or combinations thereof, and (iii) an aqueous-solute fluid.

Further, the dispersions of this disclosure are suitable for use on a variety of surfaces and substrates, including but not limited to, furniture, ingestibles, plants, trees, and the like.

It has been surprisingly found, in accordance with this disclosure, that the preparation of dispersion compositions utilizing the combination of low energy mixing followed by ultra-high energy mixing provides dispersions having reduced viscosity and enhanced stability. In the process of this disclosure, the one or more polymer or copolymer fragments, or combinations thereof, of the second dispersion exhibit at least a 10%, or 20%, or 30%, or 40%, or 50%, or 60%, or 70%, or 80%, or 90%, reduction in viscosity, as compared to the viscosity of the same one or more polymers, copolymers, or combinations thereof, of the first dispersion, under standard conditions. Also, in the process of this disclosure, the one or more polymer or copolymer fragments, or combinations thereof, of the second dispersion stabilize the particles of one or more hydrophobic agent(s) in the second dispersion for a greater period of time, when set to the same viscosity level, as compared to stabilization of the one or more hydrophobic agent(s) by the same one or more polymers, copolymers, or combinations thereof, of the first dispersion, under standard conditions. Further, in the process of this disclosure, the ultra-high energy mixing imparts a repulsive force that causes the particles of the one or more hydrophobic agent(s) to repel or move away from each other in the second dispersion, thus enhancing the stability and dispersibility of the second dispersion.

The surprising reduced viscosity and enhanced stability exhibited by the dispersions of this disclosure are attributable to several unique features including, but not limited to, the small and substantially homogeneous particle size of each hydrophobic particle, the uniform distribution or disperstivity of the hydrophobic particles minimizing the tendency of the hydrophobic particles to coalesce as would be predicted by the Ostwald Ripening equation, the hydrophobic particles possessing a net negative charge which repulse one another thereby exhibit an anti-coalescent tendency, and the polymer and copolymer fragments providing a matrix or organized fluid that reduces Newtonian flow thereby reducing the number of hydrophobic particle collisions and potential coalescence. In addition, the dispersions of this disclosure are free of surfactants.

### BRIEF DESCRIPTION OF THE DRAWINGS

FIG. 1 depicts an illustrative process flow diagram according to the present disclosure.

FIG. 2 shows the product type and hydrophobe for dispersion compositions of this disclosure, in accordance with the Examples.

FIG. 3 shows dispersion assignment tracker data generated for compositions of this disclosure, in accordance with the Examples. The data shows dispersion stability of the compositions of this disclosure.

FIG. 4 shows viscosity versus shear data generated for compositions of this disclosure, in accordance with the Examples. The data shows that putting compositions containing the polymeric and copolymeric rheology modifiers of this disclosure through ultra-high energy mixing substantially decreases the viscosity of the polymeric and copolymeric rheology modifiers.

### DETAILED DESCRIPTION OF THE EMBODIMENTS

The oil-in-water dispersions of one or more hydrophobic agent(s) of this disclosure usefully employ one or more polymer or copolymer fragments, or combinations thereof, and as a solvent, water or a mixture of water and a water miscible solvent, such as ethanol or glycerin. These compositions can contain as hydrophobic agents, aesthetic modifying agents that impart a desirable tactile, olfactory, or visual property to an animal (such as a human) skin, hair or mucosal surface to which the compositions are applied. Further, these compositions can contain as hydrophobic agents, therapeutic agents that treat disorders of human (or animal) skin, hair or mucosal tissue to which they are applied. However, as discussed above, it is because of the water-miscible solvent content of the compositions, standard emulsification practices are problematic, in particular, the stability of the emulsions is particularly problematic when the hydrophilic phase contains one or more water-miscible solvents. As a consequence, the emulsions are aesthetically unappealing to the user, and the base selections for the hydrophobic therapeutic agents are very limited.

It has now been unexpectedly found that oil-in-water dispersions of hydrophobic agents having an average particle size of about 5 m or less, polymer or copolymer fragments, or combinations thereof, and an aqueous-solute fluid, are stable for a commercially viable period of time. These dispersions of hydrophobic agents can greatly enhance the aesthetic and therapeutic properties of the composition. Further, these dispersions of hydrophobic agents can be easily diluted in the composition post-production to deliver the preset or desired level of therapeutic agents and the preset or desired aesthetic properties. When the aqueous-solute fluid is described herein, it will be recognized that the aqueous fluid or water miscible solvent can be sourced from a concentrated starting dispersion, or from materials used to dilute such a starting dispersion of particles. The compositions can easily be scaled from the laboratory to production. In addition, the compositions of this disclosure can contain one or more surface active agent(s) as a functional agent.

The compositions can contain hydrophilic aesthetic modifying agents and therapeutic agents which are believed to reside in the composition outside of the dispersion particles of hydrophobic agents.

The compositions of the present disclosure contain one or more dispersions of particles of one or more hydrophobic agents having an average particle size of about 5 m or less, and one or more polymer or copolymer fragments, or combinations thereof, in an aqueous continuous phase. Such dispersions are referred to herein as hydrophobe-in-water dispersions. Oil-in-water dispersions are examples of hydrophobe-in-water dispersions. Hydrophobe-in-water dispersions can also have, for example, silicone or Omega-3-6-9 Fatty Acids as the hydrophobes dispersed in an aqueous continuous phase.

A “hydrophobic agent” according to the disclosure has a solubility of less than about 0.1% by weight in water under standard conditions. Generally, the dielectric constant of the solvent provides a rough measure of a solvent's polarity. The strong polarity of water is indicated, at 20° C., by a dielectric constant of 80.10. Materials with a dielectric constant of less than 15 are generally considered to be nonpolar. In embodiments, the “hydrophobic agent” component(s) are substantially non-polar, in that 90% wt. or more are non-polar by this dielectric constant measure. In embodiments, 95% or 99% wt. or more of the hydrophobic agent component(s) are non-polar.

A “therapeutic agent(s)” according to this disclosure is used to treat disorders of human (or animal) skin, hair or mucosal tissue to which the compositions are applied. As used herein, the term “therapeutic agent(s)” includes pharmaceutical therapeutic agent(s).

An “aesthetic modifying agent(s)” according to this disclosure imparts a desirable tactile, olfactory, or visual property to an animal (such as a human) skin, hair or mucosal surface, or to a surface or substrate, to which the compositions are applied

A “functional agent(s)” according to this disclosure are additives, imparts a functionality (e.g., cleaning, coloring, fragrancing, styling, and the like), to human (or animal) skin, hair or mucosal tissue, or to a surface or substrate, to which the compositions are applied. Functional agent(s) include, for example, surfactants, neutralizing agents, chelating agents, foaming agents, rheological modifying agents, sensates, and the like. Surfactants can be added to the dispersions of this disclosure for cleansing or foaming purposes.

“Polymer fragment(s)” and “copolymer fragment(s)” according to this disclosure are formed by ultra-high energy mixing using, for example, a high-pressure high-shear device, sonicator, or combinations thereof. The polymer and copolymer fragments, and any derivatives or combinations thereof exhibit at least a 10%, or 15%, or 20%, or 25%, or 30%, or 35%, or 40%, or 45%, or 50%, or 55%, or 60%, or 65%, or 70%, or 75%, or 80%, or 85%, or 90%, reduction in viscosity, as compared to the viscosity of the same unfragmented polymers or copolymers, or combinations thereof, under standard conditions. The viscosity of the polymer or copolymer fragments, or combinations thereof, is less than or equal to 90%, or 85%, or 80%, or 75%, or 70%, or 65%, or 60%, or 55%, or 50%, of the same unfragmented polymers or copolymers, or combinations thereof, under standard conditions.

“Miscibility” or “miscible” according to this disclosure refers to the ability of a substance or solute (e.g., ethanol) to mix in all proportions with another substance or solvent (e.g., water), forming a homogeneous mixture or solution. “Miscibility” or “miscible” is associated with two liquids and indicates that the solvent (e.g., water) and the solute (e.g., ethanol) are soluble with each other at any ratio. As used herein, “miscibility” or “miscible” include “solubility” or “soluble”.

“Solubility” or “soluble” according to this disclosure refers to the ability of a substance or solute to form a solution with another substance or solvent (e.g., water), in which the solute has a solubility in the solvent (e.g., water) of 0.1% by weight or greater. “Solubility” or “soluble” is associated with both liquids and solids. As used herein, “solubility” or “soluble” include “miscibility” or “miscible”.

Parameters involved with solubility or the dissolution process include polarity (dielectric constant), temperature, and pressure. Assuming temperature and pressure are constant at standard temperature and pressure, then polarity is a critical parameter. This is typically measured by taking the dielectric constant of the solvent or solution.

The dielectric constant of a solvent is a measure of its polarity. The higher the dielectric constant of a solvent, the more polar it is. Polar solvents dissolve polar solutes and nonpolar solvents dissolve nonpolar solutes. The dielectric constant of a solvent can help predict how well a solute molecule will dissolve in it.

The polarity of a solute is determined by the presence of polar functional groups such as hydroxyl (—OH), carbonyl (>C═O), and amino (—NH2) groups. The polarity of a solvent is determined by its dielectric constant. A substance with a high dielectric constant is easily polarized, allowing countercharges to be placed around an ion, resulting in Coulombic interactions between solvent and ion, promoting solubilization of the ion by competing with interionic interactions.

The polarity and dielectric constant of both the solvent and solute play an important role in determining solubility. Polar solvents dissolve polar solutes and nonpolar solvents dissolve nonpolar solutes. The higher the dielectric constant of a solvent, the more polar it is, which promotes Coulombic interactions between solvent and ion, promoting solubilization of the ion by competing with interionic interactions.

An “aqueous-solute fluid” according to the disclosure can be water or a combination of water and one or more materials or solutes that have a solubility in water of 0.1% by weight or greater. As used herein, “aqueous-solute fluid” includes “polar solute”, “water miscible liquid or solute”, “water soluble liquid or solute”, and “water soluble solid or solute”.

A “polar solute” of the current disclosure is one that has a solubility in water of 0.1% by weight or greater. As used herein “polar solute” includes “water miscible solute”, and “water soluble solute”.

A “water miscible liquid or solute” of the current disclosure is one that can mix in all proportions with water, forming a homogeneous solution.

A “water soluble solid or solute” according to the disclosure is one or more materials or solutes that are solid at a temperature of 23° C. and a pressure of 100 kPa (1 bar), and have a solubility in water of 0.1% by weight or greater. For the solid solutes, the solubility product constant (Ksp) is used to represent the level at which a solute dissolves in solution. Ksp is the equilibrium constant for a solid substance dissolving in an aqueous solution. The more soluble a substance is, the higher the Ksp value it has. As used herein, Ksp is determined at a temperature of 23° C. and a pressure of 100 kPa (1 bar).

A “water soluble liquid or solute” according to the disclosure is one or more materials or solutes that are liquid at a temperature of 23° C. and a pressure of 100 kPa (1 bar), and have a solubility in water of 0.1% by weight or greater. The water soluble liquids or solutes are flowable, non-viscous, semi-viscous, or viscous liquids, at a temperature of 23° C. and a pressure of 100 kPa (1 bar).

An “aqueous fluid” according to the disclosure can be water or a combination of 50% or more water and from 0 to 50% solutes other than water miscible solutes.

“Hydrophobic agent particles” are colloidal droplets of hydrophobic agent(s), wherein at some temperature in the range of 20 to 90° C. the droplets would be liquid. A colloid is a substance microscopically dispersed throughout another substance. A colloidal system consists of two separate phases: a dispersed phase (or internal phase) and a continuous phase (or dispersion medium) in which the colloid is dispersed.

A “hydrophobic dispersion” is defined as a suspension of hydrophobic agent particles in an aqueous fluid or an aqueous-solute fluid with an average particle size of from 100 nm to 5 m. In embodiments of the disclosure, 85% or more, or 90% or more, of the hydrophobic agent particles by weight have a size within ±2.0 standard deviations, or within ±1.9 standard deviations, or within ±1.8 standard deviations, or within ±1.7 standard deviations, or within ±1.6 standard deviations, or within ±1.5 standard deviations, of the average particle size. The hydrophobic agent particles are not included in the water-solvent-solute weight percentages. The dispersion of hydrophobic agent particles can be reduced by the processes described herein, or as concentrated therefrom, or diluted therefrom.

To treat indications with a therapeutic agent or functional agent, an “effective amount” of a therapeutic agent or functional agent will be recognized by clinicians but includes an amount effective to treat, reduce, alleviate, ameliorate, eliminate or prevent one or more symptoms of the condition sought to be treated, or alternately, the condition sought to be avoided, or to otherwise produce a clinically recognizable favorable change in the condition or its effects.

A “dispersion” as used herein means, a suspension of hydrophobic agent particles having an average particle size of about 5 m or less, and polymer or copolymer fragments, or combinations thereof, in an aqueous fluid or an aqueous-solute fluid.

“Dispersion stability”, and variations thereof, refer to the ability of a dispersion to resist change in its properties over time. The changes may be physical or chemical and may be visible or invisible. For example, a lack of dispersion stability may manifest as a visible phase separation (i.e., sedimentation). Microfluidization or the small size of the dispersion hydrophobic particles (i.e., an average particle size of about 5 m or less), together with the polymer or copolymer fragments, or combinations thereof, are important for imparting dispersion stability.

An “agent” as used in this application, is a substance that brings about a chemical or physical effect or causes a chemical reaction.

A “hydrophobe” or “hydrophobic agent,” as used in this application, is a molecule or compound that is repelled by or has no attraction to water and hydrophobe has little or no solubility in water, for example less than 0.1%, less than 0.05%, or less than 0.03%. Examples include oils, alkanes, and esters of fatty acids.

An “edible” material according to this disclosure is one that is generally recognized as safe for human or animal consumption.

The dispersions of hydrophobic agent particles having an average particle size of about 5 m or less of this disclosure can be “contacted” with food products. The meaning of “contacted” will be understood by those of skill in the art, and includes being applied onto or into the food substrate using any commercially viable process.

“Food(s)” according to this disclosure is any food generally recognized for human or animal consumption including, but not limited to, meats such as chicken, turkey, beef, buffalo, pork, lamb, goat, fish, scallops, other seafood, or the like; beverages; processed foods; hydratable foods such as pastas, rice, other grains, dried fruits or vegetables (such as dried beans), drink concentrates, or the like; milk or milk substitutes; soups, sauces; grain flour; and the like.

“Sensate(s)” according to this disclosure are substances that impart a sensation to the mucous membranes, oral cavity, throat, or skin. These substances may be used as flavors or fragrances in a wide range of products such as personal care products (perfumes, deodorants, cosmetics, shampoos, skin creams, toothpastes, and the like), pharmaceuticals (such as cough syrups, cough drops and the like), and foods (such as chewing gum, soda, and the like). The sensation can be, for example, a cooling effect, a warming effect, a tingling effect, an emollient effect, and any, derivatives or combinations thereof.

“Pre-biotics” according to this disclosure are materials that can be ingested into the stomach to selectively support the growth of beneficial bacteria while reducing the ability of pathogenic bacteria to grow. These pre-biotic materials that favor the proliferation of beneficial bacteria at the expense of pathogenic microorganisms are beneficial for the maintenance of a protective barrier, proper metabolism, and maintenance of good health of the body including the skin.

“Standard condition(s)” according to this disclosure are ambient conditions, or a temperature of 23° C. and a pressure of 100 kPa (1 bar).

### Process

In accordance with the process of this disclosure, a premix is prepared comprising (i) one or more hydrophobic agent(s), (ii) an aqueous-solute fluid, (iii) one or more polymers, one or more copolymers, or combinations thereof, and optionally (iv) one or more additive(s). The premix is subjected to low energy mixing to form a first dispersion. The first dispersion is then subjected to ultra-high energy mixing to form a second dispersion. The second dispersion comprises particles of one or more hydrophobic agent(s) having an average particle size of about 5 m or less dispersed in an aqueous-solute fluid, and one or more polymer or copolymer fragments, or combinations thereof. The one or more polymer or copolymer fragments, or combinations thereof, are present in an amount sufficient to stabilize said second dispersion. The one or more polymer or copolymer fragments, or combinations thereof, are fluidized in the second dispersion.

As discussed herein, the dispersions of the present disclosure can be produced by a combination of low energy mixing and ultra-high energy mixing (e.g., high-pressure high-shear homogenization). It has been unexpectedly found by the present disclosure that the initial particle size obtained by low energy mixing, prior to ultra-high energy mixing is, in part, critical to achieving the stability of the combined continuous phase with the dispersions, together with the polymer or copolymer fragments, or combinations thereof.

Each hydrophobic material has a terminal particle size achievable by ultra-high energy mixing. The terminal particle size varies based on the material. Surfactant processes yield a different terminal particle size with undesirable chemical additives or processing conditions.

In an exemplary embodiment, prior to ultra-high energy mixing to create the dispersions of the present disclosure, an initial average particle size of the raw material components for the dispersion is on the order of several microns.

After ultra-high energy mixing, an average particle size of the components of the dispersion can be, about 200 nm, about 205 nm, about 210 nm, about 215 nm, about 220 nm, about 225 nm, about 250 nm, about 275 nm, about 300 nm, about 325 nm, about 350 nm, about 375 nm, about 400 nm, about 425 nm, about 450 nm, about 475 nm, about 480 nm, about 500 nm, about 525 nm, about 550 nm, about 575 nm, 600 nm, about 625 nm, about 650 nm, about 675 nm, about 700 nm, about 725 nm, about 750 nm, about 1000 nm, about 3000 nm, and any ranges or subranges between any of the foregoing and including endpoints.

As used herein, “low energy mixing” refers to low shear mixing in which the mixing is mechanical. Size of particles produced limited to the mechanical equipment tolerances unless significant amounts of emulsifier/surfactant are used. Illustrative mechanical equipment useful for low energy mixing includes, but not limited to, side-sweep mixing, counter-rotational mixing oars, propeller/fixed shaft+ attached mixing head (prop/paddle/saw-tooth/etc.), media mills (sand, beads, etc.), roller mills (physical rollers that can be moved closer/further from one another), homogenizer (rotor-stator set-up with variable/interchangeable stators—large holes, medium holes, small holes, slotted, square holes of variable size, diamond shaped), and the like.

As used herein, “ultra-high energy mixing” refers to ultra-high shear mixing in which the mixing is non-mechanical. Illustrative non-mechanical equipment useful for ultra-high energy mixing includes, but not limited to, microfluidizer, sonicator, and the like. A microfluidizer relies on pressure/volume (up to 30,000 psi), impingement of two fluid streams colliding with each other, forced through fixed geometry chambers with entry orifice of a larger size than the exit orifice to allow for particle expansion and diffuse distribution upon exit of chamber, discrete processing capability allows for uniform particles sizes and tight particle distributions. A microfluidizer is highly reproducible. Sonication relies on a probe/horn that translates electrical current into vibrational energy (ultrasonic waves), difficult to achieve overall homogeneity of particles produced and generally produces multiple particle size peaks, difficulties with reproducibility of particle size and distributions curves.

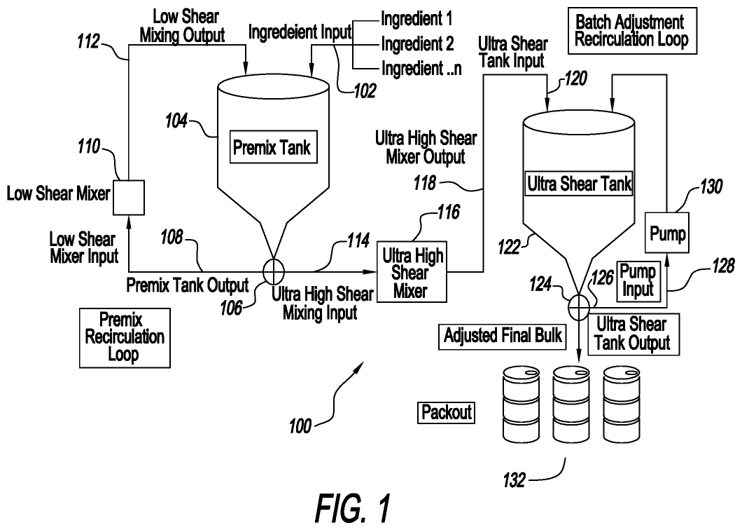

Referring to FIG. 1, in an example, the dispersions can be created by a dispersion process 100 that uses multi-step mixing, namely low energy mixing followed by ultra-high energy mixing.

The ingredients for the dispersion are fed into a premix tank 104 via ingredient input 102. The ingredients include water, or water and one or more miscible solvents, one or more hydrophobes, and optionally other additives. The premix tank 104 has a valve 106 for directing the premix tank output 108 to a low shear mixer 110, or for directing an ultra-high shear mixing input 114 to an ultra-high shear mixer 116. The premix tank output 108 is mixed with the low shear mixer 110 to a preset or desired particle size, to produce a low shear mixing output 112, which is returned to the premix tank 104 for holding or until ready for ultra-high shear mixing. This constitutes a premix recirculation loop. Nonlimiting examples of the low shear mixer 110 include, for example, propeller mixing, pump recirculation, rotor stator homogenization, media mills, and colloid mills.

In examples, a mixture of water, or water and one or more miscible solvents, one or more hydrophobes, and optionally one or more additives, are low energy mixed until an average particle size of the hydrophobes in the mixture is optimally less than 150 microns to yield a premix tank output 108 or a first dispersion. Mixing by low shear mixer 110 is mechanical and is performed at ambient pressure and temperature. Mixing is performed until an average particle size of the hydrophobes in the mixture is less than 150 microns, less than 100 microns, less than 80 microns, less than 50 microns, less than 40 microns, less than 30 microns, less than 25 microns, preferably less than 20 microns, more preferably less than 12 microns, still more preferably less than 10 microns, and most preferably less than 8 microns. Surprisingly, such low energy mechanical mixing prior to ultra-high energy mixing achieves optimal hydrophobe-in-water dispersions.

As described above, premix tank 104 has a valve 106 for directing the ultra-high shear mixing input 114 to a fluidizer or ultra-high shear mixer 116. The ultra-high shear mixing input 114 is mixed with the ultra-high shear mixer 116 to a preset or desired particle size, to produce an ultra-high shear mixer output 118, which is fed, as ultra shear tank input 120, to an ultra shear tank 122 for holding, reworking, or until ready for packaging. The ultra-high shear mixer 116 can have a heat exchanger (not shown) to maintain temperature of the ultra high shear mixer output 118. Non-limiting examples of ultra-high shear mixer 116 include, for example, high-pressure and high-shear devices, sonication, and the like.

The ultra shear tank 122 has a valve 124 for directing the ultra shear tank output 126 to packout as final bulk 132 or to a pump 130 through pump input 128. The pump output is returned to the ultra shear tank 122 for holding, further reworking, or until ready for packaging. This constitutes a batch adjustment recirculation loop.

The heat exchanger maintains temperatures in ultra high shear mixer 116 below 35 C°, preferably below 32 C°, more preferably below 29 C°, and most preferably below 27 C°.

In one example, the dispersions are produced by a combination of shear forces, impact forces, and energy dissipation forces.

Shear forces are unaligned forces that push a portion of the particle body in one specific direction, and another portion the particle body in the opposite direction. Thereby, the particles are caused to fracture and be broken up into smaller particles.

Impact forces occur when two particles collide with each other or with another object/body. Non-homogeneous particles result in inelastic collisions. Conversely, homogeneous particles result in elastic collisions and a more uniform final particle size. High velocity collisions between the particles cause the particles to exhibit a brittle behavior causing them to fracture and be broken up into smaller particles.

Dissipation forces increase the entropy of the system. Viscous forces, for example are the force that act on the particles in the direction in which the particles are moving relative to other particles and hence opposite to the direction in which the particles are moving relative to each other.

In an exemplary embodiment, dispersion process 100 will produce a dispersion of particles of the one or more hydrophobic agent(s) in which particles at least 79 wt % of the total hydrophobic particles in the dispersion are ±1.50 standard deviations of the value for the average particle size.

In exemplary embodiments, at least 79 wt % of the total hydrophobic particles in the dispersion are ±2.0 standard deviations of the value for the average particle size, preferably are ±1.75 standard deviations of the value for the average particle size, more preferably are ±1.5 standard deviations of the value for the average particle size.

In exemplary embodiments, dispersion process 100 of the present disclosure will produce a suspension of particles of the one or more hydrophobic agent(s) in which particles at least 85 wt % of the total hydrophobic particles in the dispersion are ±1.50 standard deviations of the value for the average particle size.

In exemplary embodiments, at least 85 wt % of the total hydrophobic particles in the dispersion are ±2.0 standard deviations of the value for the average particle size, preferably are ±1.75 standard deviations of the value for the average particle size, more preferably are ±1.5 standard deviations of the value for the average particle size.

In exemplary embodiments, dispersion process 100 of the present disclosure will produce a suspension of particles of the one or more hydrophobic agent(s) in which at least 87 wt % of the total hydrophobic particles in the dispersion are ±1.50 standard deviations of the value for the average particle size.

In exemplary embodiments, at least 87 wt % of the total hydrophobic particles in the dispersion are ±2.0 standard deviations of the value for the average particle size, preferably are ±1.75 standard deviations of the value for the average particle size, more preferably are ±1.5 standard deviations of the value for the average particle size.

In exemplary embodiments, dispersion process 100 of the present disclosure will produce a suspension of particles of the one or more hydrophobic agent(s) in which at least 90 wt % of the total hydrophobic particles in the dispersion are ±1.50 standard deviations of the value for the average particle size.

In exemplary embodiments, at least 90 wt % of the total hydrophobic particles in the dispersion are ±2.0 standard deviations of the value for the average particle size, preferably are ±1.75 standard deviations of the value for the average particle size, more preferably are ±1.5 standard deviations of the value for the average particle size.

In exemplary embodiments, dispersion process 100 of the present disclosure will produce a suspension of particles of the one or more hydrophobic agent(s) in which at least 93 wt % of the total hydrophobic particles in the dispersion are ±1.50 standard deviations of the value for the average particle size.

In exemplary embodiments, at least 93 wt % of the total hydrophobic particles in the dispersion are ±2.0 standard deviations of the value for the average particle size, preferably are ±1.75 standard deviations of the value for the average particle size, more preferably are ±1.5 standard deviations of the value for the average particle size.

In exemplary embodiments, dispersion process 100 of the present disclosure will produce a suspension of particles of the one or more hydrophobic agent(s) in which at least 95 wt % of the total hydrophobic particles in the dispersion are ±1.50 standard deviations of the value for the average particle size.

In exemplary embodiments, at least 95 wt % of the total hydrophobic particles in the dispersion are ±2.0 standard deviations of the value for the average particle size, preferably are ±1.75 standard deviations of the value for the average particle size, more preferably are ±1.5 standard deviations of the value for the average particle size.

Dispersion process 100 according to the present disclosure can impart a net negative charge on the particles of the dispersion. The absolute value of the negative charge can be at least −15 mV, or −32 mV, or −35 mV or greater.

The dispersions of the disclosure may be produced by mixing an aqueous fluid, polymer or copolymer fragments or combinations thereof, and hydrophobic agents using processing conditions known in the art including, but not limited to, sonication (Sonic Man, Matrical Bioscience, Spokane, WA), high pressure/high shear (e.g., utilizing Microfluidizer, Microfluidics Company, Newton, Massachusetts), freeze drying (Biochima Biophys Acta 1061:297-303 (1991)), reverse phase evaporation (Microencapsulation 16:251-256 (1999)), and bubble method (J Pharm Sci 83(3):276-280).

In sonication, for example, high intensity sound waves bombard the product for predetermined period of time. In direct sonication, the sonication probe is directly applied into the composition for processing. In indirect sonication, the composition is immersed into an ultrasonic bath, where it is exposed to the processing conditions for a predetermined period of time.

Precipitation utilizes compounds that are poorly soluble in water, but soluble in organic solvents and surfactants that are water-soluble, to create emulsions. Two separate solutions are formed, one of an organic solvent and compounds, the other a mixture of surfactant dissolved in water. The two solutions are combined, and an emulsion is created. The organic solvent is then evaporated out of the emulsion, causing the small spherical particles to precipitate, creating a suspension of particles.

High pressure/high shear utilizes an aqueous phase and a hydrophobic phase. The aqueous phase is prepared into a solution with any other water-soluble ingredients. Further, water miscible solvents are optionally added to create an aqueous-solute phase. The hydrophobic phase is prepared into a mixture with any other non-water miscible or non-water soluble components. The two phases are subjected to pressure ranging from 10,000-50,000 psi. The resulting dispersion contains suspended particles of hydrophobic agents, together with polymer or copolymer fragments, or combinations thereof.

In freeze drying, two available methods are thin film freezing and spray freeze drying. In spray freeze drying, for example, an aqueous solution containing active or therapeutic ingredients is atomized into the cold gas above a cryogenic liquid. The atomized particles adsorb onto the gas-liquid interface and aggregate there as particles.

The production process is adapted to obtain hydrophobic particles of the appropriate size. The hydrophobic agent particles of the disclosure, which are typically non-mechanically created, differ from the typical micelles whose creation is dependent on surfactant. The particles of the dispersion of the disclosure are believed to be stable primarily due to small size, rather than surfactant effects. This stability enhancement is defined by Stokes' Law which is illustrated in an equation relating the terminal settling or rising velocity of a smooth sphere in a viscous fluid of known density and viscosity to the diameter of the sphere when subjected to a known force field. This equation is V=(2 gr²)(d1−d2)/9μ, where V=velocity of fall (cm/sec), g=acceleration of gravity (cm/sec²), r=radius of particle (cm), d1=density of particle (g/cm³), d2=density of medium (g/cm³), and μ=viscosity of the medium (dyne sec/cm²). Using this equation, with all other factors being constant, a 200 nm hydrophobic agent particle has a velocity of fall that is 680 times slower than one of identical composition having a 5 micron particle size of a standard dispersion.

The dispersions of this disclosure are free of surfactants. Surfactants are defined herein to be amphiphiles or amphiphilic compounds having a Critical Micelle Concentration (CMC) greater than 10⁻⁸ mol/L or micelle forming amphiphiles or amphiphilic compounds. The dispersions according to this disclosure are free of amphiphilic compounds with a CMC greater than 10′ mol/L.

The dispersion can be created by mixing the hydrophobic agents with polymer or copolymer fragments, or combinations thereof, an aqueous fluid or an aqueous-solute fluid. The precursor form is generally of higher concentration of hydrophobic agent, and can be, without limitation, diluted with a mixture of solvent, water, and optionally a rheological modifying agent.

The composition may be produced with a shear that creates in combination with pressure an average particle size of between about 100 nm to about 5 m or less, such as between about 100-1000 nm, or 150-900 nm. The process can, for example, without limitation, include a rapid return to atmospheric pressure. Embodiments include wherein 85% or more, or 90% or more, of the particles by weight or, in other embodiments, by volume, are within one of the above-cited ranges.

Size distribution for a dispersion can be measured by a Nanotrac particle size analyzer (Microtrac, Montgomeryville, PA), or a Malvern ZetaSizer particle size analyzer (Malvern Instruments Ltd. Malvern, UK). Sizes recited herein are those determined by dynamic light scattering for spectrum analysis of Doppler shifts under Brownian Motion. Measurements are made using Mie scattering calculations for spherical particles. This reproducible methodology can be conducted with other available instruments for measuring average particle size and particle size distribution, including instruments from Horiba Scientific (Edison, NJ).

The temperature of operation used to produce the dispersion of hydrophobic agents having an average particle size of about 5 m or less is generally between about 15° C. and about 30° C. In certain embodiments, the process avoids temperatures in excess of about 50° C., or in excess of about 60° C. However certain embodiments may require a temperature exceeding 60° C. to melt the hydrophobic agent.

Without wishing to be bound by a single theory, it is believed that the non-mechanical processing, together with the polymer or copolymer fragments, or combinations thereof, imparts the stability. The dispersions offer manufacturing flexibility because the processing makes them compatible with a wide variety of base compositions, unlike conventional emulsions that require specifically tailored processing, such as chemical or heating.

The dispersions of the present disclosure are produced by a non-mechanical process that imparts a small and substantially homogeneous particle size (i.e., an average particle size of about 5 m or less) to each particle of a hydrophobic agent.

A dispersion of one or more hydrophobic agent(s) used in the present disclosure can possess a net negative charge after non-mechanical processing, such as by high-pressure high-shear processing. In one example, the absolute value of the negative charge can be at least −15 mV. In another example, my, the absolute value of the negative charge can be at least −32 mV. In yet another example, my, the absolute value of the negative charge can be at least −35 mV or greater.

Preferred methods of non-mechanical processing impart a slight repulsive force that causes the particles of the one or more hydrophobic agent(s) to repel or move away from each other in the dispersion, thus enhancing the stability and dispersibility of the dispersion.

The compositions and dispersions are mechanically and non-mechanically processed under temperature conditions different from traditional emulsions that require heat. In addition to surfactants, traditional emulsions typically also require temperatures of 70-99° C. Preparation of these traditional emulsions further require an activation energy to be achieved in addition to mechanical energy.

The compositions and dispersions are prepared under ambient temperatures. Processing can be below 50° C., below 45° C., below 40° C. and below 30° C. Advantageously, compositions and dispersions of the present disclosure require mechanical and non-mechanical energy to be formed.

Moreover, the amount of heat required for traditional emulsion preparation is unsafe for preparing compositions according to the present disclosure because for compositions containing flammable materials such as the high level of alcohol content. Alcohols have a low flashpoint and are flammable at high concentrations. For example, above about 11° C. to 15° C. ethanol or isopropanol can emanate vapors in a quantity sufficient to form an ignitable mixture with the air.

The dispersion used in the composition of the present disclosure can be non-mechanically processed until most or all particles of the hydrophobic agent(s) are sufficiently small and essentially monodispersed to be on the side of a dispersity barrier (i.e., Ostwald ripening), where a sufficient quantity of the particles are at their smallest size (critical or terminal particle size) to minimize the risk of sedimentation or creaming, and to make the dispersion stable for commercial applications.

The dispersity barrier is a different value for each hydrophobic agent and depends on the physical and chemical properties of the hydrophobic agent. The particles can also possess a net negative charge which repulse one another. The stability of the dispersion and the diffusion of the hydrophobic agent(s) throughout the aqueous continuous phase can be further enhanced when a sufficient number of particles exceed the electrostatic barrier where the magnitude of the charge creates a force of repulsion that is greater than the force on the particles to coalesce. The more particles of hydrophobic agent that exceed both the dispersity barrier and the electrostatic barrier, the greater the stability of the dispersion.

### Dispersions

The particles of the dispersions of the present disclosure can have a net negative charge so that the particles exhibit an anti-coalescent tendency. Each particle can be acted upon by a repulsive force from each surrounding particle in a 3-dimensional space or volume such as the base or initial composition.

The portion (or alternatively, the ratio) of particles that are “over” the electrostatic barrier (i.e. the point at which repulsion forces exceed the coalescing forces in the dispersion), in relation to the total number of particles, can be a measure of the stability and quality of the dispersion. The electrostatic barrier can have a different value for each hydrophobic agent and depends on the physical and chemical properties of the hydrophobic agent. However, the value of the electrostatic barrier for hydrophobic agents can fall within the same range. In addition, in some instances the value of the electrostatic barrier for a hydrophobic agent can be moved somewhat by the selection of processing conditions.

In an exemplary embodiment, at least 20 wt % of the total hydrophobic particles in the dispersion can be over the electrostatic barrier (meaning that repulsion forces exceed coalescing forces for 20 wt % of the particles), indicating that the dispersion is stable. In another preferred embodiment, 50 wt % or more of the particles can be over the electrostatic barrier, indicating that the dispersion is more stable relative to the earlier embodiment. In a preferred embodiment, 75 wt % or more of the particles can be over the electrostatic barrier, indicating that the dispersion is even more stable. In increasingly preferred embodiments, 87 wt % or more, 90 wt % or more, 95 wt % or more, and 97 wt % or more of the particles of the hydrophobic agent can be over the electrostatic barrier, respectively, indicating dispersions that are increasingly stable.

The dispersions of one or more hydrophobic agent(s) of the present disclosure can possess a net negative charge after non-mechanical processing, such as by high-pressure high-shear processing. In one example, the absolute value of the net negative charge can be 30 mV or lower. In another example, my, the absolute value of the net negative charge can be 32 mV or lower. In yet another example, my, the absolute value of the net negative charge can be 35 mV or lower.

The particle sizes in the dispersions according to the present disclosure are maintained above 100 nm by dispersion process 100 in FIG. 1, so that the average particle size is greater than 100 nm, preferably 120 nm.

Dispersions having an average particle size that is greater than 100 nm have the additional benefit of being regulatory compliant with guidelines that define nanotechnology as particles with an average particle size of less than 100 nm, i.e. that are smaller than the low end of the particle size range of the present disclosure.

The dispersions of the present disclosure containing particles of one or more hydrophobic agents having an average particle size of about 5 m or less, can be stored in a concentrated form prior to use, such as about 30 wt % to about 70 wt %. Advantageously, the concentrated dispersion can be diluted nearer to the time when it is added to the base or initial composition. For example, the concentrate can be diluted 1.5-fold, 2-fold, 5-fold, 10-fold, 50-fold, 100-fold, 200-fold, and even 1000-fold.

Dilution of the concentrate to the preset or desired concentration can be used to optimize the benefits of the dispersion for various applications (e.g., nutritional, pharmaceutical, biomedical, cosmetic, food, animal care, household, pet care, and the like). The dispersions of this disclosure can be edible dispersions.

Advantageously, the first (or second, third etc.) dispersion can be diluted to a preset or desired concentration without upsetting stability, namely without causing flocculation, Ostwald ripening, sedimentation, coalescence, creaming, and phase inversion.

The method can also include preparing a second dispersion. The second dispersion having an average particle size of about 5 m or less, can be mixed into the first dispersion having an average particle size of about 5 m or less, prior adding to the base or initial composition. Alternatively, the first and second dispersions can be added directly to the base or initial composition.

Advantageously, the first (or second, third etc.) dispersion can be mixed in various ratios without upsetting stability, namely without causing flocculation, Ostwald ripening, sedimentation, coalescence, creaming, and phase inversion.

Subjecting the mixture of components to be dispersed to one or more preparatory steps, such as low energy mixing in low shear mixer 110, can facilitate increasing the number of elastic collisions in ultra-high energy mixing in ultra high shear mixer 116 so that the particles of the hydrophobic agent are approximately the same size and mass before high-pressure high-shearing, and their elastic collision produces particles that that are smaller but remain approximately equal to each other in size and mass. The resulting particles are then analyzed for particle size, degree of monodispersity, and magnitude of the electrostatic charge. The desired properties of the particles in the dispersion are thus attained more quickly, and with less fuel, less energy, and less cost than conventional techniques, and so manufacturing is more commercially viable.

In accordance with this disclosure, a period of time for stability can be at least one month, or at least two months, or at least three months, or at least six months, or at least one year, and longer, and ranges therebetween, at standard conditions.

A commercially viable period of time for stability according to the present disclosure can be 28 days, one month, two months, three months, six months, one year, and longer, and ranges therebetween, at standard conditions.

It is the small size of the dispersion particles, together with the polymer or copolymer fragments, or combinations thereof, that imparts stability. The small size minimizes the tendency of hydrophobic particles to coalesce. The commercially viable stability described above allows a useful amount of time in which to store compositions to maintain product integrity.

The stability is further manifested in that two or more distinct dispersions can be mixed without decreasing the stability of the various component hydrophobic agent particles, or a dispersion can be diluted into aqueous fluid or aqueous-solute fluid without decreasing the stability of the component hydrophobic agent particles.

In the dispersions of the present disclosure, the hydrophobic agent(s) are sufficiently small and monodispersed to be on the side of a dispersity barrier, where a sufficient quantity of the particles are at their smallest size (critical or terminal particle size) to minimize the risk of sedimentation or creaming, and to make the dispersion stable for commercial applications. The dispersity barrier is a different value for each hydrophobic agent and depends on the physical and chemical properties of the hydrophobic agent. The particles can also possess a net negative charge which repulse one another. As described above, the stability of the dispersion and the diffusion of the hydrophobic agent(s) throughout the aqueous continuous phase can be further enhanced when a sufficient number of particles exceed the electrostatic barrier where the magnitude of the charge creates a force of repulsion that is greater than the force on the particles to coalesce. The more particles of hydrophobic agent that exceed both the dispersity barrier and the electrostatic barrier, the greater can be the stability of the dispersion.

### Hydrophobic Agents

Examples of hydrophobic agents include but are not limited to, mono, di, tri, or poly alkyl (or alkenyl) esters or ethers of a di-, tri-, or polyhydroxy compound, such as glycerin, sorbitol or other polyol compound. Examples of such esters or ethers include but are not limited to, saturated and unsaturated, linear and branched vegetable oils, such a soybean oil, almond oil, castor oil, canola oil, cottonseed oil, grapeseed oil, rice bran oil, palm oil, coconut oil, palm kernel oil, olive oil, linseed oil, sunflower oil, safflower oil, peanut oil and corn oil. Useful saturated and unsaturated oils include those having 90% or more (molar) fatty acyl components with 6 to 30 carbon atoms, such as 6 to 24 carbons, or 12 to 24 carbons.

Examples of fatty acids providing fatty acyl components, or which provide hydrophobic agents include, without limitation, for example (from www.scientificpsychic.com/fitness/fattyacids.html):

TABLE A
Common Fatty Acids

| | Carbon | Double | | |
|---|---|---|---|---|
| Common Name | Atoms | Bonds | Scientific Name | Sources |
| Butyric acid | 4 | 0 | butanoic acid | butterfat |
| Caproic Acid | 6 | 0 | hexanoic acid | butterfat |
| Caprylic Acid | 8 | 0 | octanoic acid | coconut oil |
| Capric Acid | 10 | 0 | decanoic acid | coconut oil |
| Lauric Acid | 12 | 0 | dodecanoic acid | coconut oil |
| Myristic Acid | 14 | 0 | tetradecanoic acid | palm kernel oil |
| Palmitic Acid | 16 | 0 | hexadecanoic acid | palm oil |
| Palmitoleic Acid | 16 | 1 | 9-hexadecenoic acid | animal fats |
| Stearic Acid | 18 | 0 | octadecanoic acid | animal fats |
| Oleic Acid | 18 | 1 | 9-octadecenoic acid | olive oil |
| Ricinoleic acid | 18 | 1 | 12-hydroxy-9-octadecenoic | castor oil |
| | | | acid | |
| Vaccenic Acid | 18 | 1 | 11-octadecenoic acid | butterfat |
| Linoleic Acid | 18 | 2 | 9,12-octadecadienoic acid | grape seed oil |
| Alpha-Linolenic | 18 | 3 | 9,12,15-octadecatrienoic acid | flaxseed |
| Acid | | | | (linseed) |
| (ALA) | | | | oil |
| Gamma-Linolenic | 18 | 3 | 6,9,12-octadecatrienoic acid | borage oil |
| Acid | | | | |
| (GLA) | | | | |
| Arachidic Acid | 20 | 0 | eicosanoic acid | peanut oil, |
| | | | | fish oil |
| Gadoleic Acid | 20 | 1 | 9-eicosenoic acid | fish oil |
| Arachidonic Acid | 20 | 4 | 5,8,11,14-eicosatetraenoic acid | liver fats |
| (AA) | | | | |
| EPA | 20 | 5 | 5,8,11,14,17-eicosapentaenoic | fish oil |
| | | | acid | |
| Behenic acid | 22 | 0 | docosanoic acid | rapeseed oil |
| Erucic acid | 22 | 1 | 13-docosenoic acid | rapeseed oil |
| DHA | 22 | 6 | 4,7,10,13,16,19- | fish oil |
| | | | docosahexaenoic | |
| | | | acid | |
| Lignoceric acid | 24 | 0 | tetracosanoic acid | small amounts |
| | | | | in most fats |

Fatty acyl compositions of some oils useful in the disclosure, reciting the rounded wt. percentage of some leading natural fatty acids, include without limitation the following (from www.scientificpsychic.com/fitness/fattyacids1.html):

TABLE B
Fatty Acid Compositions of Hydrophobic Agents

| | | | | | | | | Poly unsaturated | |
|---|---|---|---|---|---|---|---|---|---|
| | | Saturated | | | | | Mono | | Alpha |
| | Unsat/ | Capr. | Laur. | Mryis. | Palm. | Stear. | unsatur. | Linoleic | Linolenic |
| | Sat. | Acid | Acid | Acid | Acid | Acid | Oleic Acid | Acid (ω6) | Acid (ω3) |
| Oil or Fat | ratio | C10:0 | C12:0 | C14:0 | C16:0 | C18:0 | C18:1 | C18:2 | C18:3 |
| Almond Oil | 9.7 | — | — | — | 7 | 2 | 69 | 17 | — |
| Beef Tallow | 0.9 | — | — | 3 | 24 | 19 | 43 | 3 | 1 |
| Butterfat (cow) | 0.5 | 3 | 3 | 11 | 27 | 12 | 29 | 2 | 1 |
| Butterfat (goat) | 0.5 | 7 | 3 | 9 | 25 | 12 | 27 | 3 | 1 |
| Butterfat (human) | 1.0 | 2 | 5 | 8 | 25 | 8 | 35 | 9 | 1 |
| Canola Oil | 15.7 | — | — | — | 4 | 2 | 62 | 22 | 10 |
| Cocoa Butter | 0.6 | — | — | — | 25 | 38 | 32 | 3 | — |
| Cod Liver Oil | 2.9 | — | — | 8 | 17 | — | 22 | 5 | — |
| Coconut Oil | 0.1 | 6 | 47 | 18 | 9 | 3 | 6 | 2 | — |
| Corn Oil (Maize Oil) | 6.7 | — | — | — | 11 | 2 | 28 | 58 | 1 |
| Cottonseed Oil | 2.8 | — | — | 1 | 22 | 3 | 19 | 54 | 1 |
| Flaxseed Oil | 9.0 | — | — | — | 3 | 7 | 21 | 16 | 53 |
| Grape seed Oil | 7.3 | — | — | — | 8 | 4 | 15 | 73 | — |
| Illipe | 0.6 | — | — | — | 17 | 45 | 35 | 1 | — |
| Lard (Pork fat) | 1.2 | — | — | 2 | 26 | 14 | 44 | 10 | — |
| Olive Oil | 4.6 | — | — | — | 13 | 3 | 71 | 10 | 1 |
| Palm Oil | 1.0 | — | — | 1 | 45 | 4 | 40 | 10 | — |
| Palm Olein | 1.3 | — | — | 1 | 37 | 4 | 46 | 11 | — |
| Palm Kernel Oil | 0.2 | 4 | 48 | 16 | 8 | 3 | 15 | 2 | — |
| Peanut Oil | 4.0 | — | — | — | 11 | 2 | 48 | 32 | — |
| Safflower Oil* | 10.1 | — | — | 1 | 7 | 2 | 13 | 78 | — |
| Sesame Oil | 6.6 | — | — | — | 9 | 4 | 41 | 45 | — |
| Shea nut | 1.1 | — | 1 | — | 4 | 39 | 44 | 5 | — |
| Soybean Oil | 5.7 | — | — | — | 11 | 4 | 24 | 54 | 7 |
| Sunflower Oil* | 7.3 | — | — | — | 7 | 5 | 19 | 68 | 1 |
| Walnut Oil | 5.3 | — | — | — | 11 | 5 | 28 | 51 | 5 |

*Not high-oleic variety

The hydrophobic agents can be colorants, such as for example annatto oil, paprika oil, chlorophyll, lycopene, carotenoids. xanthophylls or the like. The hydrophobic agents can be essential nutrients, such as for example, vitamins such as Vitamin D and its derivatives, Vitamin A and its derivatives, Vitamin E and its derivatives, Vitamin K, Vitamin F, Vitamin P, and the like. Other such nutrients include for example lipoic acid, lycopene, phospholipids, ceramides, ubiqinone, sterols, flavonoids, cholesterol, sphingolipids, prostaglandins, docosahexaenoic acid, and the like.

The hydrophobic agents can be fragrances or flavors, such as for example terpenes, isoterpenenes, alkyl lactones, essential oils, natural oils such as vanilla, and the like. The hydrophobic agents can be aroma providers that impart aroma to or modify aroma of a topical composition.

The hydrophobic agents (including aesthetic modifying agents if present) can be present in the dispersion composition in an amount of 0.01% wt. to 70%, or 0.1% wt. to 70%, or 5% to 65%, or 0.2% wt. to 60%, or 10% to 60%, by wt., or 0.3% wt. to 55%, or 0.4% wt. to 50%, based on the total weight of the dispersion composition.

The hydrophobic agents can include aesthetic modifying agents or therapeutic agents. Therapeutic agents and aesthetic modifying agents can include hydrophobic agents and hydrophilic agents.

For example, 0.01% wt. to 70%, or 0.1% wt. to 70%, or 0.5% to 65%, or 0.2% wt. to 60%, or 1% to 60%, or 0.3% wt. to 55%, by wt., based on the total weight of the dispersion composition, can be hydrophobic agents.

In embodiments, the skin, hair or mucosal composition can be for example 0.01% wt. to 70% wt, or 0.1% wt. to 65%, or 0.5% to 60%, or 0.25% wt. to 55%, or 1% to 50%, based on the total weight of the composition, of hydrophobic agents.

As used herein, therapeutic agents can be hydrophobic, in which case they will associate with the hydrophobic agent particles, or hydrophilic, in which case they will associate with the aqueous-solute fluid.

Suitable therapeutic agents, both hydrophobic and hydrophilic, include, but are not limited to, anti-acne agents, antimicrobial agents, anti-inflammatory agents, analgesics, anti-erythemal agents, anti-pruritic agents, anti-edemal agents, anti-psoriatic agents, anti-fungal agents, skin protectants, sunscreen agents, vitamins, antioxidants, scavengers, anti-irritants, anti-bacterial agents, antiviral agents, antiaging agents, photoprotection agents, hair growth enhancers, hair growth inhibitors, hair removal agents, antidandruff agents, anti-seborrheic agents, exfoliating agents, wound healing agents, anti-ectoparasitic agents, sebum modulators, immunomodulators, hormones, botanicals, moisturizing agents, hand sanitizing agents, astringents, sensates, antibiotics, anesthetics, steroids, tissue healing substances, tissue regenerating substances, amino acids, peptides, minerals, ceramides, biohyaluronic acids, skin bleaching ingredients, and any derivatives or combinations of the foregoing.

Suitable therapeutic agents that are anti-acne agents include, but are not limited to, salicylic acid, retinoic acid, alpha hydroxy acid, benzoyl peroxide, sodium sulfacetamide, clindamycin, hydrocortisone, tetrahydrozoline, and any derivatives or mixtures thereof.

Suitable therapeutic agents that are antimicrobial agents include, but are not limited to, benzalkonium chloride, benzethonium chloride, chlorhexidine gluconate, chloroxylenol, clindamycin, cloflucarban, erythromycin, fluorosalan, hexachlorophene, hexylresorcinol, iodine complex, iodine tincture, para-chloromercuriphenol, phenylmercuric nitrate, thimerosal, vitromersol, zyloxin, triclocarban, triclosan, methyl-benzethonium chloride, nonyl phenoxypoly (ethyleneoxy) ethanol-iodine, para-chloro-meta-xylenol, providone-iodine complex, poloxamer-iodine complex, undecoylium chloride-iodine complex, and any derivatives or combinations of the foregoing.

Suitable therapeutic agents that are anti-inflammatory agents include, but are not limited to, alidoxa, allantoin, aloe vera, aluminum acetate, aluminum hydroxide, bismuth subnitrate, boric acid, calamine, casein, microporous cellulose, cholecalciferol, cocoa butter, cod liver oil, colloidal oatmeal, cysteine hydrochloride, dexpanthenol, dimethicone, glycerin, alpha-bisabolol, sea whip extract, glycyrrhetinic acid and its salts and derivatives, kaolin, lanolin, live yeast cell derivative, mineral oil, Peruvian balsam, petrolatum, protein hydrolysate, racemethionine, shark liver oil, sodium bicarbonate, sulfur, talc, tannic acid, topical starch, vitamin A, vitamin E, white petrolatum, zinc acetate, zinc carbonate, zinc oxide, hydrocortisone, betamethasone, ibuprofen, indomethacin, acetylsalicylic acid, tacrolimus, fluocinolone acetonide, sodium sulfacetamide, and any derivatives or combinations of the foregoing.

Suitable therapeutic agents that are analgesics include, but are not limited to, diphenhydramine, tripelennamine, benzocaine, dibucaine, lidocaine, tetracaine, camphor, menthol, phenol, resorcinol, matacresol, juniper tar, methylsalicylate, turpentine oil, capsicum, methyl nicotinate, beta-glucan, and any derivatives or combinations of the foregoing.

Suitable therapeutic agents that are anti-erythemal agents include, but are not limited to, tetrahydrozoline and hydrocortisone, and any derivatives or combinations of the foregoing.

Suitable therapeutic agents that are antipruritic agents include, but are not limited to, diphenhydramine, pramoxine, antihistamines, and any derivatives or combinations of the foregoing.

Suitable therapeutic agents that are anti-edema agents, include, but are not limited to, pregnenolone acetate, tannin glycosides, and any derivatives or combinations of the foregoing.

Suitable therapeutic agents that are antipsoriatic agents include, but are not limited to, calcipotriene, coal tar, anthralin, vitamin A, hydrocortisone, retinoic acid, alpha hydroxy acid, dovonex, salicylic acid, sunscreen agents, indomethacin, urea; anthralin, and any derivatives or combinations of the foregoing.

Suitable therapeutic agents that are antifungal agents include, but are not limited to, clioquinol, haloprogin, miconazole nitrate, clotrimazole, metronidazole, tolnaftate, undecylenic acid, iodoquinol, and any derivatives or combinations of the foregoing.

Suitable therapeutic agents that are skin protectants include, but are not limited to, cocoa butter, dimethicone, petrolatum, white petrolatum, glycerin, shark liver oil, allantoin, and any derivatives or combinations of the foregoing.

Suitable therapeutic agents that are sunscreen agents or active pharmaceutical ingredients (APIs) include, but are not limited to, ethylhexyl methoxycinnamate, avobenzone, benzophenones, octocrylene, ethylhexyl salicylate, homomenthyl salicylate, triethanolamine salicylate, menthyl anthranilate, PABA, octyl dimethyl para amino acid PABA, 2-ethoxyethyl p-methoxycinnamate, phenylbenzimidazole sulfonic acid, titanium dioxide, zinc oxide, and any derivatives or combinations of the foregoing.

The one or more sunscreen active agents provide adsorption or blocking of UV radiation, before it reaches the skin. Illustrative sunscreen active agents include, for example, avobenzone, cinoxate, dioxybenzone, homosalate, menthyl anthranilate, octocrylene, octisalate, octyl methoxycinnamate, octyl salicylate, oxybenzone, padimate O, phenylbenzimidazole sulfonic acid, sulisobenzone, titanium dioxide, trolamine salicylate, zinc oxide, benzophenone-3, ethylhexyl methoxycinnamate, octocrylene, butyl methoxydibenzoylmethane (BMBM), diethylamino hydroxybenzoyl hexyl benzoate, diethylhexyl butamido triazone, PABA, camphor benzalkonium methosulfate, phenylbenzimidazole sulfonic acid, terephthalidene dicamphor sulfonic acid, benzylidene camphor sulfonic acid, polyacrylamidomethyl benzylidene camphor, PEG-25 PABA, isoamyl p-methoxycinnamate, ethylhexyl triazone, drometrizole trielloxane, 4-methylbenzylidene camphor, 3-benzylidene camphor, ethylhexyl salicylate, ethylhexyl dimethyl PABA, benzophenone-4, methylene bis-benztriazolyl tetramethylbutylphenol, disodium phenyl dibenzimidazole tetrasulfonate, bis-ethylhexyloxyphenol methoxyphenol triazine, methylene bisbenzotriazolyl tetramethylbutylphenol, bisethylhexyloxyphenol methoxyphenyl triazine, and any combination thereof.

In an embodiment, the sunscreen active agent is selected from homosalate, octocrylene, avobenzone, octisalate, ethylhexyl methoxycinnamate, butyl methoxydibenzoylmethane (BMBM), diethylamino hydroxybenzoyl hexyl benzoate, diethylhexyl butamido triazone, or any combination thereof.

In another embodiment, the sunscreen active agent comprises homosalate, octocrylene, ethylhexyl methoxycinnamate, butyl methoxydibenzoylmethane (BMBM), diethylamino hydroxybenzoyl hexyl benzoate, and diethylhexyl butamido triazone.

Approved sunscreen active agents in the United States and elsewhere include, for example, paraaminobenzoic acid, avobenzone, cinoxate, dioxybenzone, homosalate, menthyl anthranilate, octocrylene, octyl methoxycinnamate, octyl salicylate, oxybenzone, 2-ethylhexyl 4-(dimethylamino)benzoate (e.g., Padimate O), phenylbenzimidazole sulfonic acid, octisalate, sulisobenzone, trolamine salicylate, titanium dioxide, and zinc oxide. Several other sunscreen active or therapeutic ingredients are accepted for use in other countries. Examples from outside the United States include Tinosorb M, Tinosorb S, Uvinul T-150, UVA sorb HEB, Uvinul A Plus, Neo Heliopan AP, and Neo Heliopan MBC.

Suitable therapeutic agents that are antioxidants include, but are not limited to, scavengers for lipid free radicals and peroxyl radicals, quenching agents, astaxanthin, tocopherol, butylated hydroxytoluene (BHT), beta carotene, vitamin A, ascorbic acid and aliphatic derivatives, ubiquinol, ferulic acid, azelaic acid, thymol, catechin, sinapic acid, ethylenediaminetetraacetic acid (EDTA), lactoferrin, rosmariquinone, hydroxytyrosol, sesamol, 2-thioxanthine, nausin, malvin, carvacone, chalcones, glutathione isopropyl ester and other aliphatic derivatives, xanthine, melanin, guanisone, loporphyrins, 8-hydroxyxanthine, 2-thioxanthione, vitamin B12, plant alkaloids, catalase, quercetin, superoxide dismutase (SOD), cysteine, methionine, genistein, nordihydroguaiaretic acid (NDGA), procyanidin, hamamelitannin, ubiquinone, trolox, licorice extract, propyl gallate, and any derivatives or combinations of the foregoing.

Suitable therapeutic agents that are vitamins include, but are not limited to, vitamin E, vitamin A palmitate, vitamin D, vitamin F, vitamin B6, vitamin B3, vitamin B12, vitamin C (ascorbic acid or water soluble derivatives of ascorbic acid), ascorbyl palmitate, vitamin E acetate, biotin, niacin, dl-panthenol, and any derivatives or combinations of the foregoing.

As described herein, the therapeutic agents can be hydrophobic, in which case they will associate with the hydrophobic agent particles, or hydrophilic, in which case they will associate with the aqueous-solute fluid.

In embodiments, hydrophobic therapeutic agents comprise 60% wt. or less of the hydrophobic agent dispersion composition.

Examples of aesthetic modifying agents include without limitation C2-C26 alkyls substituted with 2-24 hydroxyls, where all of the hydroxyls of the foregoing compounds are independently acylated with a saturated, unsaturated, linear, branched or cyclic C1-C24 alkane. In embodiments, the substituted C2-C26 alkyls are reduced sugars (i.e., of the general formula CᵢH₂ᵢ₊₂Oₙ).

An example of a hydrophobic agent is a compound having the formula A:

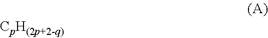

where p is an integer greater than or equal to 6 and q is 0 or an even integer no greater than p. Such compounds include, but are not limited to, saturated and unsaturated, linear, branched, cyclic hydrocarbon chains. Examples of such compounds include without limitation mineral oil, petrolatum, permethyl fluids, polybutenes, polyisobutenes, and any derivatives or mixtures thereof.

Another example of a hydrophobic aesthetic modifying agent has formula B:

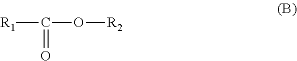

or formula C:

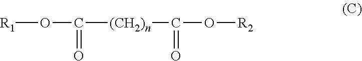

where R₁ is a saturated or unsaturated, linear, branched or cyclic C1-C23 acyl moiety having 0, 1, or more substituent groups; R₂ is hydrogen or a saturated or unsaturated, liner, branched or cyclic C1-C24 acyl moiety having 0, 1, or more substituent groups; and n is an integer from 0 to 20. Examples of such aesthetic modifying agents include, but are not limited to, isopropyl palmitate and diisopropyl adipate.

Another example of a hydrophobic aesthetic modifying agent has formula D:

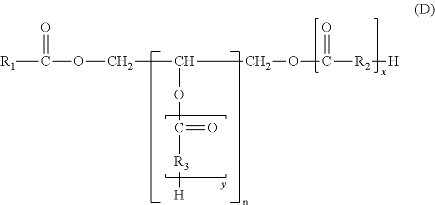

wherein R1 is a C₆ to C₂₄ acyloate group comprising saturated, unsaturated, cyclic, branched, substituted, oxidized, reduced, polymeric, or copolymeric hydrocarbon(s); R2 and R3 are independently a C₃ to C₂₄ acyloate group comprising saturated, unsaturated, cyclic, branched, substituted, oxidized, reduced, polymeric, or copolymeric hydrocarbon(s), less 1 hydrogen at the omega carbon; x is a value of 0 or 1; y is a value of 0 to n; and n is a value of 1 to 6.

Still another aesthetic modifying agent is silicone. Silicone may provide lubrication and/or shine to the formulation. Preferably, the silicone is insoluble in water. Suitable water-insoluble silicone materials include, but are not limited to, polysiloxanes, cyclic siloxanes, polyalkylsiloxanes, polyarylsiloxanes, polyalkylarylsiloxanes, polysiloxane gums, polyethersiloxane copolymers, and silicone crosspolymers. Examples of suitable silicone materials are disclosed in U.S. Pat. Nos. 4,788,006; 4,341,799; 4,152,416; 3,964,500; 3,208,911; 4,364,837 and 4,465,619, all of which are incorporated herein by reference for their teachings on silicone materials.

Another suitable hydrophobic material which can be suspended in the formulation has formula E:

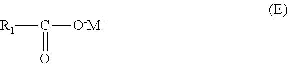

wherein M⁺ is N⁺R₃R₄R₅R₆; wherein R₃, R₄, and R₅, are each independently hydrogen or a saturated or unsaturated, linear or branched alkane or hydroxyalkane group having from 1 to 10 carbon atoms; and R₆ is a saturated or unsaturated, linear, branched or cyclic alkyl or substituted alkane group having 2 to 24 carbon atoms. An example of such a material is dimethyl lauramine oleate.

### Polymer and Copolymer Fragments, and any Derivatives or Combinations Thereof.

The polymer and copolymer fragments, and any derivatives or combinations thereof as used in this disclosure exhibit at least a 10%, or 15%, or 20%, or 25%, or 30%, or 35%, or 40%, or 45%, or 50%, or 55%, or 60%, or 65%, or 70%, or 75%, or 80%, or 85%, or 90%, reduction in viscosity, as compared to the viscosity of the same unfragmented polymers or copolymers, or combinations thereof, under standard conditions. The viscosity of the polymer or copolymer fragments, or combinations thereof, is less than or equal to 90%, or 85%, or 80%, or 75%, or 70%, or 65%, or 60%, or 55%, or 50%, of the same unfragmented polymers or copolymers, or combinations thereof, under standard conditions. In accordance with this disclosure, the polymer and copolymer fragments, and any derivatives or combinations thereof are formed by ultra-high energy mixing using, for example, a high-pressure high-shear device, sonicator, or combinations thereof.

The molecular weight and size of the polymer and copolymer fragments, and any derivatives or combinations thereof, can vary over a wide range depending on the particular polymers and copolymers. The polymer and copolymer fragments are polydisperse in that they contain polymer and copolymer fragment chains of unequal length, and so the molecular weight and size are not simple values. The polymer or copolymer fragments exist as a distribution of chain lengths and molecular weights. The molecular weight of the polymer and copolymer fragments is described as an average molecular weight calculated from the molecular weights of all the polymer and copolymer fragment chains. The molecular weight averages can be described as number average molecular weight (Mn) and weight average molecular weight (Mw). These molecular weight averages can be determined by gel permeation chromatography (GPC) and size exclusion chromatography (SEC), particle sizers such as a Zetasizer for particles less than 1250 nm and a Mastersize for particles from 1000 nm to 50,000 nm.

The number average molecular weight (Mn) is a statistical average molecular weight of all the polymer and copolymer fragment chains in a sample, and can be determined as follows:

$$Mn = \frac{\sum {N}_{i} {M}_{i}}{\sum {N}_{i}}$$

wherein Mᵢ is the molecular weight of a polymer or copolymer fragment chain and Nᵢ is the number of polymer or copolymer fragment chains of that molecular weight. Mn can be predicted by polymerization mechanisms and is measured by methods that determine the number of polymer or copolymer fragment chains in a sample of a given weight, for example, colligative methods such as end-group assay. If Mn is quoted for molecular weight distribution, there are equal number of polymer and copolymer fragment chains on either side of Mn in the distribution.

The polymer and copolymer fragments, and any derivatives or combinations thereof, as used in this disclosure, exhibit at least a 5%, or 10%, or 25%, or 40%, or 45%, or 50%, or 60%, or 75%, or greater, reduction in Mn, as compared to the Mn of the same unfragmented polymers or copolymers, or combinations thereof, under standard conditions.

The weight average molecular weight (Mw) can be determined as follows:

$$Mw = \frac{\sum {N}_{i} {M}_{i}^{2}}{\sum {N}_{i} {M}_{i}}$$

wherein Mᵢ and Nᵢ are as described above. Compared to Mn, Mw takes into account the molecular weight of a polymer and copolymer fragment chains in determining contributions to the molecular weight average. The more massive the polymer or copolymer fragment chain, the more the polymer or copolymer fragment chain contributes to molecular weight. Mw is determined by methods that are sensitive to molecular size rather than just their number, such as light scattering techniques. If Mw is quoted for a molecular weight distribution, there is an equal weight of polymer or copolymer fragment chains on either side of Mw in the distribution.

The polymer and copolymer fragments, and any derivatives or combinations thereof as used in this disclosure exhibit at least a 5%, or 10%, or 25%, or 40%, or 45%, or 50%, or 60%, or 75%, or greater, reduction in Mw, as compared to the Mw of the same unfragmented polymers or copolymers, or combinations thereof, under standard conditions.

The polydispersity index (PI) or dispersity index (DI) is a measure of the heterogeneity of polymer and copolymer sizes in a mixture, and can be determined as follows:

$$Polydispersity index = \frac{Mw}{Mn}$$

wherein Mw and Mn are as described above. PI values less than 0.05 are more common to monodisperse samples (where all the polymer and copolymer chain lengths are equal), while values greater than 0.7 are common to broad size (i.e., polydisperse) distribution of polymer and copolymer chain lengths.

The polymer and copolymer fragments, and any derivatives or combinations thereof as used in this disclosure exhibit at least a 5%, or 10%, or 15%, or 20%, or 25%, greater PI, as compared to the PI of the same unfragmented polymers or copolymers, or combinations thereof, under standard conditions.

The polymer and copolymer fragments, and any derivatives or combinations thereof as used in this disclosure have an average size less than about 100 nm.

The one or more polymer or copolymer fragments, or combinations thereof, are sufficient to stabilize the particles of one or more hydrophobic agent(s) having an average particle size of about m or less, in the dispersions, at a level from about 0.05% w/w to about 70% w/w, or from about 0.1% w/w to about 70% w/w, or from about 0.5% w/w to about 70% w/w, or from about 1% w/w to about 70% w/w, or from about 5% w/w to about 70% w/w, or from about 10% w/w to about 70% w/w, of the one or more hydrophobic agent(s), in the dispersions. Weight/weight equivalents can be calculated as known in the art.

### Aqueous-Solute Fluids

The aqueous-solute fluid according to the disclosure can be water or a combination of water and one or more materials or solutes that have a solubility in water of 0.1% by weight or greater. Aqueous-solute fluids include, for example, water miscible liquids or solutes, water soluble liquids or solutes, and water soluble solids or solutes.

Illustrative water soluble liquids or solutes useful in this disclosure include, for example, glyceraldehyde, erythrose, erythrulose, sedoheptulose, and the like.

Illustrative water soluble solids or solutes useful in this disclosure include, for example, carbohydrates selected from monosaccharides, reduced sugar alcohols, sugar acids, substituted monosaccharides, disaccharides, triglycerides, and polysaccharides (glycans); amino acids, peptides, and proteins; vitamins; minerals; and any derivatives or combinations thereof.

Illustrative monosaccharides include, for example, 1, 3-dihydroxy-2-propanone, arabinose, ribose, xylose, lyxose, ribulose, xylulose, psicose, sorbose, tagatose, threose, erythrulose, glucose, fructose, mannose, galactose, allose, altrose, gulose, indose, talose, and dihydroxacetone.

Illustrative reduced sugar alcohols include, for example, threitol, arabitol, xylitol, ribitol, mannitol, sorbitol, galactitol, fucitol, iditol, inositol, volemitol, isomalt, maltitol, and lactitol.

A sugar acid or acidic sugar is a monosaccharide with a carboxyl group at one end, or the other end, or both ends of its chain. Illustrative sugar acids include, for example, aldonic acids, ulosonic acids, uronic acids, and aldaric acids. With aldonic acids, the aldehyde group (—CHO) located at the initial end (position 1) of an aldose is oxidized. With ulosonic acids, the —CH₂(OH) group at the initial end of a 2-ketose is oxidized creating an α-ketoacid. With uronic acids, the —CH₂(OH) group at the terminal end of an aldose or ketose is oxidized. With aldaric acids, both ends (—CHO and —CH₂(OH)) of an aldose are oxidized.

Other examples of sugar acids include aldonic acids such as glyceric acid, xylonic acid, gluconic acid, threonic acid, and ascorbic acid; ulosonic acids such as neuraminic acid (5-amino-3,5-dideoxy-D-glycero-D-galacto-non-2-ulosonic acid), and ketodeoxyoctulosonic acid (KDO or 3-deoxy-D-manno-oct-2-ulosonic acid); uronic acids such as glucuronic acid, galacturonic acid, lactobionic acid, and iduronic acid; and aldaric acids such as tartaric acid, meso-galactaric acid (mucic acid), and D-glucaric acid (saccharic acid).

Still other examples of sugar acids include N-acetylneuraminic acid, N-acetyltalosaminuronic acid, aldonic acid, 3-deoxy-D-manno-oct-2-ulosonic acid, N-glycolylneuraminic acid, hexenuronic acid, isosaccharinic acid, lactobionic acid, muramic acid, pangamic acid, sialic acid, threonic acid, ulosonic acid, and uronic acid.

Illustrative substituted monosaccharides useful in this disclosure include, for example, sugar esters including phosphate sugar esters, amino sugar esters, acylate sugar esters, and any derivatives or combinations thereof.

Illustrative phosphate sugar esters include, for example, glucose-1-phosphate, fructose-1,6-diphosphate, and any derivatives or combinations thereof.

Illustrative amino sugar esters include, for example, 2-glucosamine, 2-galactosamine, N-acetylglucosamine, N-acetylmannosamine, neuraminic acid, N-acetyltalosaminuronic acid, and any derivatives or combinations thereof.

Illustrative acylate sugar esters include, for example, methyl-glucoside, muramic acid, N-acetyl-neuraminic acid, N-glycosyl-neuraminic acid, pangamic acid, and any derivatives or combinations thereof.

Illustrative disaccharides useful in this disclosure include, for example, sucrose (fructose-glucose), lactose (galactose-glucose), maltose (glucose-glucose), isomaltose, maltobiose, trehalose, cellobiose, and any derivatives or combinations thereof.

Illustrative triglycerides useful in this disclosure include, for example, raffinose (glucose-fructose-galactose), melizitose, and any derivatives or combinations thereof.

Illustrative polysaccharides (glycans) useful in this disclosure include, for example, starch, glycogen, amylopectin, amylose, cellulose, dextran, chitan, alginic acid, agarose, glycosylaminoglycans including chondroitin sulfate, heparin, hyaluronic acid, dermatan sulfate, keratan sulfate, ascorbic acid (vitamin C), the j-D form of glucuronic acid, and any derivatives or combinations thereof.

Illustrative amino acids, peptides, and proteins useful in this disclosure include, for example: alpha-amino acids including alanine, arginine, asparagine, aspartate, cysteine, glutamate, glutamine, glycine, histidine, isoleucine, leucine, lysine, methionine, phenylalanine, proline, serine, threonine, tryptophan, valine, selenocysteine, pipecolic acid, pyrrolysine, and any derivatives or combinations thereof; beta-amino acids including beta-alanine, beta-aminoisobutyric acid, and any derivatives or combinations thereof; gamma-amino acids including gamma-amino butyric acid, carnitine, and any derivatives or combinations thereof; dipeptides including cysteinyl-thionine, glycyl-glycine, alanyl-histidine, cysteinyl-glycine, and any derivatives or combinations thereof; tripeptides including glutathione, glycyl-glycyl-glycine, lysyl-lysyl-lysine, glutamyl-histeinyl-glycine, and any derivatives or combinations thereof; substituted amino acids and peptides including acetyl carnitine, acetyl cysteine, methylglycinate, glutathione methyl ester, and any derivatives or combinations thereof; proteins including enzymes, cytokines, growth factors, structural proteins such as collagen, elastin, keratin, and any derivatives or combinations thereof.

Illustrative vitamins useful in this disclosure include, for example: vitamin C (ascorbic acid or water soluble derivatives of ascorbic acid), vitamin B1 (thiamin), vitamin B2 (riboflavin), vitamin B3 (niacin), vitamin B5 (pantothenic acid), vitamin B6 (pyridoxin), vitamin B7 (biotin), vitamin B9 (folate), vitamin B12 (cobalamin), and any derivatives or combinations thereof.

Illustrative minerals useful in this disclosure include, for example: aluminum bromide, aluminum chlorate, aluminum chloride, aluminum nitrate, aluminum sulfate, ammonium acetate, ammonium bromide, ammonium carbonate, ammonium chlorate, ammonium chloride, ammonium fluoride, ammonium hydrogen carbonate, ammonium iodide, ammonium nitrate, ammonium phosphate, ammonium sulfate, ammonium sulfide, ammonium sulfite, barium acetate, barium bromide, barium chlorate, barium chloride, barium hydroxide, barium iodide, barium nitrate, barium nitrite, calcium acetate, calcium bromide, calcium chlorate, calcium chloride, calcium iodide, calcium nitrate, calcium nitrite, cobalt (III) acetate, cobalt (III) bromide, cobalt (III) chlorate, cobalt (III) chloride, cobalt (III) iodide, cobalt (III) nitrate, cobalt (III) sulfate, copper (II) acetate, copper (II) bromide, copper (II) chlorate, copper (II) chloride, copper (II) fluoride, copper (II) nitrate, copper (II) sulfate, iron (II) acetate, iron (II) bromide, iron (II) chloride, iron (II) iodide, iron (II) nitrate, iron (II) sulfate, iron (III) bromide, iron (III) chloride, iron (III) iodide, iron (III) nitrate, iron (III) sulfate, lead (II) acetate, lead (II) chlorate, lead (II) nitrate, lead (II) nitrite, lithium acetate, lithium bromide, lithium carbonate, lithium chlorate, lithium chloride, lithium fluoride, lithium hydrogen carbonate, lithium hydroxide, lithium iodide, lithium nitrate, lithium nitrite, lithium sulfate, lithium sulfide, lithium sulfite, magnesium acetate, magnesium bromide, magnesium chlorate, magnesium chloride, magnesium iodide, magnesium nitrate, magnesium nitrite, magnesium sulfate, magnesium sulfite, nickel acetate, nickel bromide, nickel chlorate, nickel chloride, nickel fluoride, nickel iodide, nickel nitrate, nickel sulfate, potassium acetate, potassium bromide, potassium carbonate, potassium chlorate, potassium chloride, potassium fluoride, potassium hydrogen carbonate, potassium hydroxide, potassium iodide, potassium nitrate, potassium nitrite, potassium phosphate, potassium sulfate, potassium sulfide, potassium sulfite, silver chlorate, silver fluoride, silver nitrate, sodium acetate, sodium bromide, sodium carbonate, sodium chlorate, sodium chloride, sodium fluoride, sodium hydrogen carbonate, sodium hydroxide, sodium iodide, sodium nitrate, sodium nitrite, sodium phosphate, sodium sulfate, sodium sulfide, sodium sulfite, zinc acetate, zinc bromide, zinc chlorate, zinc chloride, zinc fluoride, zinc iodide, zinc nitrate, zinc sulfate, and any derivatives or combinations thereof.

Illustrative water miscible liquids or solutes useful in this disclosure include, for example, acetaldehyde, acetic acid, acetone, 1,2-butylene glycol, 1,3-butylene glycol, 1,4-butylene glycol, 2-butoxyethanol, dimethyl sulfoxide, ethanol, ethoxydiglycol, triethylene glycol, ethylene glycol, methanol, isopropanol, 1,2-propylene glycol, 1,3-propylene glycol, 1-propanol, propanoic acid, diglycerin, polyglycerol, glycerin, 1.5-pentylene glycol, hexylene glycol, and any derivatives or combinations thereof.

Other illustrative water miscible liquids or solutes include, for example, one or more of liquids or solutes according to formula E:

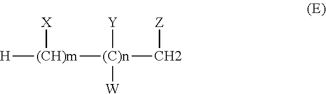

where X, Y and Z are independently —H or —OH; W is independently —H or —CH₃; m is 0 or 1; and n is an integer from 0 to 6. For example, the liquids or solutes can be mono, di, tri, tetra or penta alcohols, such as, but not limited to, methanol, ethanol, isopropyl alcohol, propanol, butanol, ethylene glycol, propylene glycol, butylene glycol, pentylene glycol, glycerol, tetritol, pentitol, 1,3 propane diol, and the like, or mixtures thereof. In embodiments, the liquids or solutes are, but are not limited to, one or more ethanol, propanol, butylene glycol, glycerol, 1,3-propane diol, or mixtures thereof. In embodiments, the liquid or solute is ethanol.

Illustrative alcohols include organic compounds that carry at least one hydroxyl functional group (—OH) bound to a saturated carbon atom. Examples include mono, di, tri, tetra or penta alcohols. Examples further include ethanol or isopropanol. Examples still further include methanol, ethanol, isopropanol, propanol, butanol, 1-octanol (capryl alcohol), caprylyl alcohol, ethylene glycol, propylene glycol, butylene glycol, pentylene glycol, hexylene glycol, glycerol or glycerin, tetritol, pentitol, 1,3 propane diol, and the like, or mixtures thereof.

The water-miscible liquid or solute of the aqueous-solute fluid can have, without limitation, concentrations greater than 10%, or 20%, or 30% wt. in the aqueous-solute fluid, or less than 95%, 90%, or 80%, or 70% wt, or a range therebetween. The water of the aqueous-solute fluid can have, without limitation, concentration greater than 4.99%, or 10%, or 20%, or 30% wt. in the aqueous-solute fluid, or less than 89.99%, or 80%, or 70% wt, or a range therebetween.

In an embodiment, in the aqueous-solute fluids of this disclosure, at least 10%, or at least 15%, or at least 20%, or at least 25%, of the water soluble liquids comprise water miscible liquids.

In reciting that a composition of the disclosure has a given percentage of water-miscible liquid or solute, it will be recognized that during formulation that total amount of the water miscible liquid or solute can be contributed from (i) a concentrated dispersion of particles of hydrophobic agent(s) having an average particle size of about 5 m or less, (ii) a separate aqueous-solute fluid that may be mixed with the concentrated dispersion, or (iii) both. Similarly, the water can come from either or both sources.

The aqueous-solute fluid is present in an amount from about 20% wt. to about 99% wt., or amount from about 25% wt. to about 95% wt., or amount from about 30% wt. to about 90% wt., or amount from about 35% wt. to about 85% wt., based on the total weight of the composition.

### Rheological Modifying Agents

The dispersions can optionally include a rheological modifying agent. Such agents are known in the art and include, but are not limited to, those set forth at www.foodadditives.org/food_gums/common.html.

Suitable rheological modifying agents include, but are not limited to, phosphorylated starch derivative, carbohydrate based rheological modifying agents, polymeric and copolymeric rheological modifying agents, inorganic rheological modifying agents, protein rheological modifying agents, polypeptide rheological modifying agents, and any derivatives or combinations of the foregoing. In accordance with this disclosure, the polymer and copolymer fragments, and any derivatives or combinations thereof, include fragments of polymeric and copolymeric rheological modifying agents. Rheological modifying agents can be added to the premix, and undergo low energy mixing and ultra-high energy mixing. Also, rheological modifying agents can be added to the second dispersion, after ultra-high energy mixing, for thickening purposes.

Examples of a phosphorylated starch derivative include, but are not limited to, starches containing a phosphate group. Suitable phosphorylated starch derivatives include, but are not limited to, hydroxyalkyl starch phosphates, hydroxyalkyl distarch phosphates, and any combination of any of the foregoing. Non-limiting examples of hydroxyalkyl starch phosphates and hydroxyalkyl distarch phosphates include: hydroxyethyl starch phosphate, hydroxypropyl starch phosphate, hydroxypropyl distarch phosphate (including sodium hydroxypropyl starch phosphate), and any derivatives or combinations of the foregoing.

Non-limiting examples of suitable carbohydrate based rheological modifying agents include algin and derivatives and salts thereof (such as algin, calcium alginate, propylene glycol alginate, and ammonium alginate); carrageenan (Chondrus crispus) and derivatives and salts thereof (such as calcium carrageenan and sodium carrageenan); agar; cellulose and derivatives thereof (such as carboxymethyl hydroxyethylcellulose, cellulose gum, hydroxyethylcellulose, hydroxypropylcellulose, hydroxypropyl methylcellulose, and ethylcellulose); chitosan and derivatives and salts thereof (such as hydroxypropyl chitosan, carboxymethyl chitosan, and chitin); gellan gum; guar (Cyamopsis tetragonoloba) and derivatives thereof (such as guar hydroxypropyltrimonium chloride and hydroxypropyl guar); hyaluronic acid and derivatives thereof (such as sodium hyaluronate); dextran and derivatives thereof; dextrin; locust bean (Ceratonia siliqua) gum; starches (such as starch polyacrylonitrile copolymer-potassium salt and starch polyacrylonitrile copolymer-sodium salt); pectin; sclerotium gum; tragacanth (Astragalus gummifer) gum; xanthan gum and derivatives thereof; and any derivatives or combinations of the foregoing.

Non-limiting examples of suitable polymeric and copolymeric rheological modifying agents include acrylates, methacrylates, acrylamides, vinyls, polyethylene and derivatives thereof, and any combination of any of the foregoing. Suitable acrylates and methacrylates include, but are not limited to, carbomer and derivatives and salts thereof, acrylates/C10-C30 alkyl acrylate crosspolymer, acrylates/ceteth-20 itaconate copolymer, acrylates/ceteth-methacrylate copolymers, acrylates/steareth-methacrylate copolymers, acrylates/steareth-20 itaconate copolymers, acrylates/steareth-50 acrylate copolymers, acrylates/VA crosspolymers, acrylates/vinyl isodecanoate crosspolymers, acrylic acid/acrylonitrogen copolymers, ammonium acrylates/acrylonitrogen copolymers, glyceryl polymethacrylate, polyacrylic acid, PVM/MA decadiene crosspolymer, sodium acrylate/vinyl isodecanoate crosspolymers, sodium carbomer, ethylene/acrylic acid copolymer, ethylene/VA copolymer, acrylates/acrylamide copolymer, acrylate copolymers, acrylates/hydroxyester acrylate copolymers, acrylate/octylarylamide copolymers, acrylates/PVP copolymers, AMP/acrylate copolymers, butylester of PVM-MA copolymer, carboxylate vinyl acetate terpolymers, diglycol/CHDM/isophthalates/SIP copolymer, ethyl ester of PVM-MA copolymer, isopropyl ester of PVM-MA copolymer, octylacrylamide/acrylate/butylaminoethyl methacrylate copolymers, polymethacrylamidopropyltrimonium chloride, polyvinylcaprolactam, PVP, PVP/dimethylaminoethylmethacrylate copolymer, PVP/DMAPA acrylate copolymers, PVP/carbamyl polyglycol ester, PVP/VA copolymer, PVP/VA vinyl propionate copolymer, PVP/vinylcaprolactam/DMAPA acrylate copolymers, sodium polyacrylate, VA/butyl maleate/isobornyl acrylate copolymers, VA/crotonates copolymer, VA/crotonates vinyl neodecanoate copolymer, VA/crotonates/vinyl propionate copolymer, vinyl caprolactam/PVP/dimethylaminoethylmethacrylate copolymer, hydroxyethyl acrylate/sodium acryloyldimethy taurate copolymer, and any derivatives or combinations of the foregoing.

Non-limiting examples of suitable inorganic rheological modifying agents include clays and derivatives thereof, silicates, silicas and derivatives thereof, and any combination of any of the foregoing. Suitable clays and derivatives thereof include, but are not limited to, bentonite and derivatives thereof, such as quaternium-18 bentonite; hectorite and derivatives thereof, such as quaterniums; montmorillonite; and any derivatives or combinations of the foregoing. Suitable silicates include, but are not limited to, magnesium aluminum silicate, sodium magnesium silicate, lithium magnesium silicate, tromethamine magnesium aluminum silicate, and any derivatives or combinations of the foregoing. Suitable silicas and derivatives thereof include, but are not limited to, hydrated silica, hydrophobic silica, spherical silica, and any derivatives or combinations of the foregoing.

Suitable protein and polypeptide rheological modifying agents include, but are not limited to, proteins and derivatives and salts thereof, polypeptides and derivatives and salts thereof, and any combination of any of the foregoing. Non-limiting examples of protein and polypeptide rheological modifying agents include albumin, gelatin, keratin and derivatives thereof, fish protein and derivatives thereof, milk protein and derivatives thereof, wheat protein and derivatives thereof, soy protein and derivatives thereof, elastin and derivatives thereof, silk protein and derivatives thereof, and any derivatives or combinations of the foregoing.

Particularly suitable rheological modifying agents include, but are not limited to, carbomer, acrylate/alkyl acrylate crosspolymers, acrylate/vinyl isododecanoate crosspolymer, xanthan gum, hydroxyethyl cellulose, locust bean gum, guar gum, and any combination of any of the foregoing. A suitable combination of rheological modifying agents comprises carbomer and an acrylate/alkyl acrylate copolymer, such as an acrylates/C10-C30 alkyl acrylate crosspolymer. According to the International Cosmetic Ingredient Dictionary and Handbook (7th ed., The Cosmetic, Toiletry, and Fragrance Association), carbomer is a homopolymer of acrylic acid crosslinked with an allyl ether of pentaerythritol, an allyl ether of sucrose, or an allyl ether of propylene. The term “acrylate/alkyl acrylate crosspolymer” includes, but is not limited to, copolymers of alkyl acrylates with one or more monomers of acrylic acid, methacrylic acid, or one of their short chain (i.e. C1-4 alcohol) esters, wherein the crosslinking agent is, for example, an allyl ether of sucrose or pentaerytritol. Preferably, the alkyl acrylates are C10-C30 alkyl acrylates. Examples of such copolymers include, but are not limited to, those commercially available as Ultrez-21, Ultrez-20, Carbopol™ 1342, Carbopol™ 1382, Pemulen™ TR-1, and Pemulen™ TR-2, from Novion, Cleveland, Ohio.

Particularly suitable rheological modifying agents include, but are not limited to, hydrophilic gelling agents, such as carboxyvinyl polymers (carbomer), acrylic copolymers (e.g., acrylate/alkyl acrylate copolymers), polyacrylamides, hydroxyethyl acrylate/sodium acryloyldimethyl taurate copolymers, polysaccharides (e.g. hydroxypropylcellulose), natural gums (e.g., xanthan gum), clays, and any derivatives or combinations of the foregoing.

Examples of fatty acids providing fatty acyl components, or which provide hydrophobic agents include, without limitation, for example: Butyric acid, Caproic Acid, Caprylic Acid, Capric Acid, Lauric Acid, Myristic Acid, Palmitic Acid, Palmitoleic Acid, Stearic Acid, Oleic Acid, Ricinoleic acid, Vaccenic Acid, Linoleic Acid, Alpha-Linolenic Acid (ALA), Gamma-Linolenic Acid (GLA), Arachidic Acid, Gadoleic Acid, Arachidonic Acid (AA), EPA, Behenic acid, Erucic acid, DHA, and Lignoceric acid.

Fatty acyl compositions of some oils useful in the invention, include without limitation: Almond Oil, Beef Tallow, Butterfat (cow), Butterfat (goat), Butterfat (human), Canola Oil, Cocoa Butter, Cod Liver Oil, Coconut Oil, Corn Oil (Maize Oil), Cottonseed Oil, Flaxseed Oil, Grape seed Oil, Illipe, Lard (Pork fat), Olive Oil, Palm oil, Palm Olein, Palm Kernel Oil, Peanut Oil, Safflower Oil, Sesame Oil, Shea nut, soybean Oil, Sunflower Oil, Walnut Oil.

The rheological modifying agent can be present in the composition or in the dispersion of hydrophobic agents in an amount from 0.01 to 10% wt, or 0.1 to 5%, or 0.2 to 2%. Rheological modifying agents are added in particular to help immobilize the particles of hydrophobic agents for still longer-term stability of the dispersions.

### Humectants

The dispersions can optionally include a humectant. Humectants are materials that bind water through hydrogen bonding. Humectants generally have multiple hydroxyl groups or amino groups. Mono, di, and poly carbohydrate or reduced carbohydrate molecules are particularly good humectants. Three-carbon trihydroxy compounds like glycerin are also particularly good.

Most 5 carbon and 6 carbon mono-, di, and poly saccharide will bind water. Glucose, ribose, fructose, xylose, xylitol, mannitol, and sucrose will bind water which is released into dry skin to improve its functionality. Polysaccharides composed of a long-chain of either a mono-carbohydrate, di-carbohydrates, or poly-carbohydrates are also suitable humectants. Glycogen, hyaluronic acid, honey, and chondroitin sulfate are examples of Poly carbohydrates that can complex copious amount of water. They release moisture into the skin to keep it hydrated and supple for extended periods. Other examples of humectants are proteins, amino acids, and ammonium lactate. The aforementioned humectants are exemplary and not intended to be limiting.

The humectant can be present in the composition or in the dispersion of hydrophobic agents in conventional amounts, for example, an amount from 0.01 to 10% wt, or 0.1 to 5%, or 0.2 to 2%.

### Edible Hydrophobic Agents

Examples of edible hydrophobic agents include but are not limited to mono, di, tri, or poly alkyl (or alkenyl) esters or ethers of a di, tri, or polyhydroxy compound, such as glycerin, sorbitol or other polyol compound. Examples of such esters or ethers include but are not limited to, saturated and unsaturated, linear and branched vegetable oils, such a soybean oil, almond oil, castor oil, canola oil, cottonseed oil, grapeseed oil, rice bran oil, palm oil, coconut oil, palm kernel oil, olive oil, linseed oil, sunflower oil, safflower oil, peanut oil and corn oil. Useful saturated and unsaturated oils include those having 90% or more (molar) fatty acyl components with 6 to 30 carbon atoms, such as 6 to 24 carbons, or 12 to 24 carbons.

Examples of fatty acids providing fatty acyl components, or which provide hydrophobic agents include, without limitation, for example those listed in Table A above.

Fatty acyl compositions of some oils useful in the invention, reciting the rounded wt percentage of some leading natural fatty acids, include without limitation those listed in Table B above.

In embodiments, without limitation, about 51% wt or more of the edible hydrophobic agent(s) are one or more of the oils identified above. In embodiments, without limitation, about 51% wt or more of the edible hydrophobic agent(s) are avocado oil, canola oil, corn oil, cottonseed oil, flaxseed oil, grape seed oil, peanut oil, safflower oil, sesame oil, soybean oil, sunflower oil, walnut oil, olive oil, peppermint oil, orange oil or a mixture thereof.

The edible hydrophobic agents can be present in the hydrophobic agent dispersion composition in an amount of 0.01% wt. to 70%, or 5% to 65%, or 10% to 60%, by weight.

### Edible Polymer and Copolymer Fragments, and any Derivatives or Combinations Thereof

Edible polymers are useful in a variety of applications, for example, food, biomedicine and cosmetics. Edible polymers refer to the polymeric materials which can be consumed by human beings, animals or microorganisms without any noxious effects towards health. They can be generally categorized into polysaccharides, proteins and lipids. The edible polymers have gained increasing application mainly in functional food industries (food packaging and nutrients protection) and biomedical fields.

Illustrative characteristics of edible polymers include the following: they can be consumed by humans and animals without any noxious health effects; polysaccharides, lipids and proteins are three main sources of edible polymers(EP); food, biomedicine and cosmetics are main applications of EP based products; EP can reduce contamination and produce eco-friendly/recyclable materials; and nanotechnology and functional additives are used in smart/active EP based products.

Edible polymers are mainly composed of polysaccharides, proteins and lipids, are nature based materials, which can be easily consumed by animals and human without any harmful effect on health. With the attractive features of the edible polymers such as biodegradability and bio/environmental compatibility over non-biodegradable synthetic polymers, these polymers have found wide-spread applications in food, agricultural industries, and biomedical fields. Edible polymers can be used to extend the shelf life of food, achieve maximum therapeutics outcome through the development of innovative edible electronics, sensors, delivery systems, and green cosmetics.

The main sources of edible polymers are polysaccharides, lipids (particularly C10 or greater saturated alkyl linear esters or ethers), and proteins. Illustrative lipids include, for example, waxes such as beeswax, candelilla, carnauba wax; phospholipids, fatty acids, triglycerides, glycolipids, and the like. Illustrative polysaccharides include, for example, starch, pectin, agar, alginate, cellulose derivatives, carrageenan, chitosan, xanthan gum, guar gum, gum Arabic, pullulan, and the like. Illustrative proteins include, for example, albumin, collagen, gelatin, milk, zein, wheat gluten, soy, peanut, pea, nut proteins, and the like.

The edible polymer and copolymer fragments, and any derivatives or combinations thereof as used in this disclosure exhibit at least a 10%, or 15%, or 20%, or 25%, or 30%, or 35%, or 40%, or 45%, or 50%, or 55%, or 60%, or 65%, or 70%, or 75%, or 80%, or 85%, or 90%, reduction in viscosity, as compared to the viscosity of the same unfragmented edible polymers or edible copolymers, or combinations thereof, under standard conditions. The viscosity of the edible polymer or copolymer fragments, or combinations thereof, is less than or equal to 90%, or 85%, or 80%, or 75%, or 70%, or 65%, or 60%, or 55%, or 50%, of the same unfragmented edible polymers or copolymers or combinations thereof, under standard conditions. In accordance with this disclosure, the edible polymer and copolymer fragments, and any derivatives or combinations thereof are formed by ultra-high energy mixing using, for example, a high-pressure high-shear device, high-pressure sonicator, or combinations thereof.

The molecular weight and size of the edible polymer and copolymer fragments, and any derivatives or combinations thereof, can vary over a wide range depending on the particular polymers and copolymers. The edible polymer and copolymer fragments are polydisperse in that they contain edible polymer and copolymer fragment chains of unequal length, and so the molecular weight and size are not simple values. The edible polymer and copolymer fragments exist as a distribution of chain lengths and molecular weights. The molecular weight of the edible polymer and copolymer fragments is described as an average molecular weight calculated from the molecular weights of all the edible polymer and copolymer fragment chains.

The edible polymer and copolymer fragments, and any derivatives or combinations thereof as used in this disclosure exhibit at least a 5%, or 10%, or 25%, or 40%, or 45%, or 50%, or 60%, or 75%, or greater, reduction in Mn, as compared to the Mn of the same unfragmented edible polymers or edible copolymers, or combinations thereof, under standard conditions.

The edible polymer and copolymer fragments, and any derivatives or combinations thereof as used in this disclosure exhibit at least a 5%, or 10%, or 25%, or 40%, or 45%, or 50%, or 60%, or 75%, or greater, reduction in Mw, as compared to the Mw of the same unfragmented edible polymers or edible copolymers, or combinations thereof, under standard conditions.

The edible polymer and copolymer fragments, and any derivatives or combinations thereof as used in this disclosure exhibit at least a 5%, or 10%, or 15%, or 20%, or 25%, greater PI, as compared to the PI of the same unfragmented edible polymers or edible copolymers, or combinations thereof, under standard conditions.

The edible polymer and copolymer fragments, and any derivatives or combinations thereof as used in this disclosure have an average size less than about 5000 nm.

The one or more edible polymer or copolymer fragments, or combinations thereof, are sufficient to stabilize the particles of one or more edible hydrophobic agent(s) having an average particle size of about 5 m or less in the dispersions, at a level from about 0.05% w/w to about 70% w/w, or from about 0.1% w/w to about 70% w/w, or from about 0.5% w/w to about 70% w/w, or from about 1% w/w to about 70% w/w, or from about 5% w/w to about 70% w/w, or from about 10% w/w to about 70% w/w, in the dispersions. Weight/weight equivalents can be calculated as known in the art.

### Edible Rheological Modifying Agents

The dispersion can optionally include edible rheological modifying agents. Such agents are known in the art and include, without limitation, those set forth in the following Table C adapted from www.foodadditives.org/food_gums/common.html.

TABLE C
Rheological Agents

Agar-agar - a gum consisting of two repeating units of polysaccharides: alpha-D-
galactopyranosyl and 3,6-anhydro-alpha-L-glactopyranosyl derived from red seaweed.
Traditional agar-agar can bind approximately 100 times its weight in water when boiled,
forming a strong gel that is often used as a stabilizer or thickener. A recent application of agar-
agar is replacing gelatin as the gelling agent in dairy products, such as yogurt. Agar-agar is a
non-animal gel source which is suitable for vegetarians and people with religious dietary
restrictions (Kosher/Halal).
Alginate - is a polysaccharide, like starch and cellulose, and is derived from brown seaweed.
Alginate provides properties in processed foods and beverages such as gelling, viscosifying,
suspending and stabilizing. Alginate gelling may be achieved using calcium under controlled
conditions. It employs the combination of alginate, a slowly soluble calcium salt and a suitable
calcium sequestrant, such as a phosphate or citrate. The process may be performed at neutral
or acid pH.
Carrageenan - a water soluble gum derived from red seaweeds, such as Eucheuma, Gigartina,
and Chondrus. Carrageenan is a sulfated linear polysaccharide of D-galactose and it has a
strong negative charge, thereby allowing it to stabilize gels or act as a thickener. Carrageenan
is found in numerous products, ranging from toothpaste to soy milk. It is used to suspend cocca
solids in beverages, for example, and can be used in meats to reduce cooking losses.
Cassia Gum - is a naturally occurring galactomannan found in the endosperm of cassia tora
and obtusifolia seeds. It is an effective thickener and stabilizer for a broad range of food
applications. Cassia gum has excellent retort stability and forms strong synergistic gels with
other hydrocolloids including carrageenan and xanthan gum. Human food grade cassia gum is
specially processed to meet rigorous purity standards.
Cellulose Gum - Carboxymethyl Cellulose (CMC), or cellulose gum is an abundant and
natural polysaccharide found in all plants. Cellulose gum is a water-soluble gum that is based
on cellulose. Cellulose gum has been used in food products for over 50 years as a thickener
and stabilizer. Typical uses are in instant beverages, where it provides texture, baked goods,
where it prevents staling, and ice-cream, where it prevents the formation of ice-crystals that
can be formed from frequent freezing and rethawing.
Gellan Gum - a food gum that is primarily used as a gelling or thickening agent. It can be used
in fortified beverages to suspend protein, minerals, vitamins, fiber and pulp. Gellan gum also
suspends milk solids in diluted milk drinks. Gellan gum can act as a fluid gel, having a wide
range of textures, and can exist as a light pourable gel or a thick, spreadable paste. Gellan gum
is a non-animal gel source which is suitable for vegetarians and people with religious dietary
restrictions (Kosher/Halal).
Guar Gum - a carbohydrate consisting of mannose and galactose at a 2:1 ratio that can swell
in cold water. Guar gum is one of the most highly efficient water-thickening agents available
to the food industry and is widely used as a binder and volume enhancer. Its high percentage
of soluble dietary fiber (80 to 85%), means that it is often added to bread to increase its soluble
dietary fiber content. Guar gum is also commonly used to thicken and stabilize salad dressings
and sauces and help improve moisture retention in finished baked goods.
Hydroxypropyl cellulose - cellulose is an abundant and natural polysaccharide found in all
plants. Hydroxypropyl cellulose is based on cellulose and is used in many food products to
provide good foam stability. Hydroxypropyl cellulose is commonly found in whipped toppings
where it stabilizes the foam and provides a long lasting whipped topping with dairy-like eating
quality.
Konjac Gum- a polysaccharide from a plant known as elephant yam, which is commonly
found in Asia. This gum can be used as a vegan substitute for gelatin and other thickeners. Its
texture makes it ideal for jellies because of its high viscosity.
Locust Bean Gum - also called Carob bean gum, locust bean gum is derived from the seeds
of the carob bean. Locust bean gum is used for thickening, water-binding, and gel
strengthening in a variety of foods. It has synergistic interactions with other gums, such as
xanthan or carrageenan, and can be used in applications such as dairy, processed cream cheese,
and dessert gels.
Methylcellulose and Hydroxypropyl Methylcellulose - cellulose is an abundant and natural
polysaccharide found in all plants. Methylcellulose and hydroxypropyl methylcellulose are
based on cellulose and are used in many food products to provide texture, certain mouth feels
and other desirable qualities. These gums are commonly found in soy burgers where they add
meat-like texture to the vegetable proteins, in fried appetizers like mozzarella cheese sticks
and onion rings where they create firm texture by reducing the uptake of frying oils, and in
whipped toppings where they stabilize the foam structure to give long lasting creams.
Microcrystalline cellulose (MCC) - is a polysaccharide derived from naturally occurring
cellulose similar to that found in fruits and vegetables. MCC can be used as a bulking agent,
source of fiber and moisture regulator in processed foods. MCC may also be co-processed with
carboxymethyl cellulose (CMC) to impart shear-thinning and heat stable properties.
Additional properties in food and beverages from MCC/CMC co-processed products include
gelling, viscosifying, suspending and stabilizing.
Pectin - a polysaccharide derived from plant material, mainly citrus fruit peels, apple peels, or
sugar beets. Pectin is widely used to impart gel formation, thickening, and physical stability to
a wide range of foods. It is mostly used in fruit-based products, including jams, jellies,
confectioneries, and fruit drinks, but is also used in dairy applications such as drinking and
spoonable yogurt.
Xanthan Gum - a highly branched polysaccharide of D-glucose, D-mannose, and D-
glucuronic acid produced via bacterial fermentation using nutrient sources. Xanthan gum,
which is considered natural, is an excellent emulsion stabilizer in salad dressings and sauces
and also is used in bakery fillings to prevent water migration from the filling to the pastry
(which has strong water-binding properties). Xanthan gum can often be used to improve the
shelf life of a product.

The edible rheological modifying agent can be present in the composition or in the dispersion of edible hydrophobic agents in an amount from 0.01 to 10% wt, or 0.1 to 5%, or 0.2 to 2%. Edible rheological modifying agents are added in particular to help immobilize the particles of edible hydrophobic agents for still longer-term stability of the dispersions.

### Adjuvants and Additives

The hydrophobic agent dispersion composition can contain suitable adjuvants which may include, but are not limited to, pH adjusters, emollients, conditioning agents, chelating agents, colorants, fragrances, flavors, odor masking agents, non-dispersed actives, UV stabilizers, preservatives, neutralizing agents, surfactants, and any combination of any of the foregoing. Suitable pH adjusters include, but are not limited to, aminomethyl propanol, aminomethylpropane diol, triethanolamine, citric acid, sodium hydroxide, acetic acid, potassium hydroxide, lactic acid, and any combination of any of the foregoing. Suitable conditioning agents include, but are not limited to, cyclomethicone, petrolatum, dimethicone, dimethiconol, silicone, quaternary amines, and any combination of any of the foregoing. The formulation can for example contain less than about 4.0% by weight of preservatives, based upon weight of total formulation, or from about 0.25% to about 3% by weight of preservatives, based upon weight of total formulation.

The rheological modifying adjuvants can be present in the composition or in the dispersion of hydrophobic agents in conventional amounts, for example, an amount from 0.01 to 10% wt, or 0.1 to 5%, or 0.2 to 2%.

### Delivery Vehicle for Active or Therapeutic Ingredients

In accordance with this disclosure, each dispersion can provide a multifunctional delivery vehicle for active or therapeutic ingredients, including one or more: anti-acne agents, antimicrobial agents, anti-inflammatory agents, analgesics, anti-erythemal agents, antipruritic agents, antiedemal agents, anti-psoriatic agents, antifungal agents, skin protectants, sunscreen agents, vitamins, antioxidants, anti-irritants, anti-bacterial agents, antiviral agents, antiaging agents, photoprotection agents, exfoliating agents, wound healing agents, sebum modulators, immunomodulators, hormones, botanicals, moisturizing agents, hand sanitizing agents, astringents, sensates, antibiotics, anesthetics, steroids, tissue healing substances, tissue regenerating substances, amino acids, peptides, minerals, ceramides, hyaluronic acids, skin bleaching ingredients, pre-biotics, probiotics, hemp oils, cannabinoids, and any derivatives or combinations of the foregoing. Advantageously, the dispersions provide such a multifunctional delivery vehicle without the need for conventional surfactants or heating which can alter or damage actives.

Anti-acne agents include, but are not limited to, salicylic acid, retinoic acid, alpha hydroxy acid, benzoyl peroxide, sodium sulfacetamide, clindamycin, hydrocortisone, tetrahydrozoline, and any derivatives or mixtures thereof.

Antimicrobial agents include, but are not limited to, benzalkonium chloride, benzethonium chloride, chlorhexidine gluconate, chloroxylenol, clindamycin, cloflucarban, erythromycin, fluorosalan, hexachlorophene, hexylresorcinol, iodine complex, iodine tincture, para-chloromercuriphenol, phenylmercuric nitrate, thimerosal, vitromersol, zyloxin, triclocarban, triclosan, methyl-benzethonium chloride, nonyl phenoxypoly (ethyleneoxy) ethanol-iodine, para-chloro-meta-xylenol, providone-iodine complex, poloxamer-iodine complex, undecoylium chloride-iodine complex, and any derivatives or combinations of the foregoing.

Anti-inflammatory agents include, but are not limited to, alidoxa, allantoin, aloe vera, aluminum acetate, aluminum hydroxide, bismuth subnitrate, boric acid, calamine, casein, microporous cellulose, cholecalciferol, cocoa butter, cod liver oil, colloidal oatmeal, cysteine hydrochloride, dexpanthenol, dimethicone, glycerin, alpha-bisabolol, sea whip extract, glycyrrhetinic acid and its salts and derivatives, kaolin, lanolin, live yeast cell derivative, mineral oil, peruvian balsam, petrolatum, protein hydrolysate, racemethionine, shark liver oil, sodium bicarbonate, sulfur, talc, tannic acid, topical starch, vitamin A, vitamin E, white petrolatum, zinc acetate, zinc carbonate, zinc oxide, hydrocortisone, betamethasone, ibuprofen, indomethacin, acetylsalicylic acid, tacrolimus, fluocinolone acetonide, sodium sulfacetamide, and any derivatives or combinations of the foregoing.

Analgesics include, but are not limited to, diphenhydramine, tripelennamine, benzocaine, dibucaine, lidocaine, tetracaine, camphor, menthol, phenol, resorcinol, matacresol, juniper tar, methylsalicylate, turpentine oil, capsicum, methyl nicotinate, beta-glucan, and any derivatives or combinations of the foregoing.

Anti-erythemal agents include, but are not limited to, tetrahydrozoline and hydrocortisone, and any derivatives or combinations of the foregoing.

Antipruritic agents include, but are not limited to, diphenhydramine, pramoxine, antihistamines, and any derivatives or combinations of the foregoing.

Anti-edema agents include, but are not limited to, pregnenolone acetate, tannin glycosides, and any derivatives or combinations of the foregoing.

Anti-psoriatic agents include, but are not limited to, calcipotriene, coal tar, anthralin, vitamin A, hydrocortisone, retinoic acid, alpha hydroxy acid, dovonex, salicylic acid, sunscreen agents, indomethacin, urea; anthralin, and any derivatives or combinations of the foregoing.

Antifungal agents include, but are not limited to, clioquinol, haloprogin, miconazole nitrate, clotrimazole, metronidazole, tolnaftate, undecylenic acid, iodoquinol, and any derivatives or combinations of the foregoing.

Skin protectants include, but are not limited to, cocoa butter, dimethicone, petrolatum, white petrolatum, glycerin, shark liver oil, allantoin, and any derivatives or combinations of the foregoing.

Sunscreen agents include, but are not limited to, ethylhexyl methoxycinnamate, avobenzone, benzophenones, octocrylene, ethylhexyl salicylate, homomethyl salicylate, triethanolamine salicylate, menthyl anthranilate, PABA, octyl dimethyl PABA, 2-ethoxyethyl p-methoxycinnamate, phenylbenzimidazole sulfonic acid, titanium dioxide, zinc oxide, and any derivatives or combinations of the foregoing.

Antioxidants include, but are not limited to, scavengers for lipid free radicals and peroxyl radicals, quenching agents, astaxanthin, tocopherol, butylated hydroxytoluene (BHT), beta carotene, vitamin A, ascorbic acid and aliphatic derivatives, ubiquinol, ferulic acid, azelaic acid, thymol, catechin, sinapic acid, ethylenediaminetetraacetic acid (EDTA), lactoferrin, rosmariquinone, hydroxytyrosol, sesamol, 2-thioxanthine, nausin, malvin, carvacone, chalcones, glutathione isopropyl ester and other aliphatic derivatives, xanthine, melanin, guanisone, loporphyrins, 8-hydroxyxanthine, 2-thioxanthione, vitamin B12, plant alkaloids, catalase, quercetin, superoxide dismutase (SOD), cysteine, methionine, genistein, nordihydroguaiaretic acid (NDGA), procyanidin, hamamelitannin, ubiquinone, trolox, licorice extract, propyl gallate, and any derivatives or combinations of the foregoing.

Vitamins include, but are not limited to, vitamin E, vitamin A palmitate, vitamin D, vitamin F, vitamin B6, vitamin B3, vitamin B12, vitamin C (ascorbic acid or water soluble derivatives of ascorbic acid), ascorbyl palmitate, vitamin E acetate, biotin, niacin, dl-panthenol, and any derivatives or combinations of the foregoing.

Sensates include, but are not limited to, menthol, isopulegole, 3-(1-menthoxy)propan-1 2-diol, p-menthan-3,8-diol, 6-isopropyl-9-methyl-1,4-dioxaspiro-(45)-decane-2-methanol, menthyl succinate, alkaline earth salts of menthyl succinate, trimethyl cyclohexanol, N-ethyl-2-isopropyl-5-methylcyclohexane carboxamide, 3-(1-menthoxy)-2-methyl-propan-1,2-diol, mint oil, peppermint oil, wintergreen, menthone, menthone glycerin ketal, menthyl lactate, [1′R,2′S,5′R]-2-(5′-methyl-2′-(meithylethyl)cyclohexyloxy)ethan-1-ol, [1′R,2′S 5′R]-3-(5′˜metlyl-2′-(methylethyl)cyclohexyloxy)propan-1-ol, [1′R,2′S 5′R]-4-(5′-metlyl-2′-(methylethyl)cyclohexyloxy)butan-1-ol, spearmint, and any derivatives or mixtures thereof. Other sensates include, for example, vanillyl ethyl ether, vanillyl propyl ether, vanillin propylene glycol acetal, ethyl vanillin propylene glycol acetal, capsaicin, gingerol, vanillyl butyl ether, 4-(I-methoxy-methyl)-2-pheny)-1-1,3-dioxolane, 4-(I-menthoxy-rethyl)-2-(3′,4′-dihydroxy-phenyl)-1,3-dioxolane, 4-(I-menthoxy-methyl)-2-(2′-hydroxy-3′-methoxy-phenyl)-1,3-dioxolane, 4-(I-menthoxy-methyl-2-(4′-methoxyphenyl)-1,3-dioxolane, 4-(I-menthoxy-methyl)-2-(3′4′ methylenedioxy-phenyl)-1,3-dioxolane, hot pepper oil, capsicum oleoresin, ginger oleoresin, nonyl acid vanillylamine, 4-(1-menthoxy-methyl)-2-(3′-methoxy-4′-hydroxyphenyl)-1,3-dioxolane, and any derivatives or mixtures thereof. Still other sensates include, for example, Jambu Oleoresin, Zanthoxylum peperitum saanshool-I, saanshool II, sanshoamide, Spilanthol, and any derivatives or mixtures thereof.

Pre-biotics include, but are not limited to, inulin, chicory, resistant starches, pectin, and any derivatives or mixtures thereof. Pre-biotics are typically polymers of three carbon, four carbon, five carbon, and six carbon carbohydrates. When pre-biotics are broken down by microorganisms, different short-chain fatty acids are created depending on the kind of pre-biotic. As a result, these short-chain fatty acids do a number of thing like provide energy, and aid in controlling inflammation and enhancing immunity.

Hemp oils and cannabinoids include, but are not limited to, cannabidiol (CBD), delta-9-tetrahydrocannabinol (THC), cannabinol (CBN), cannabigerol (CBG), cannabichromene oil (CBC), any hemp oil or cannabinoid species (e.g., sativa, indica, ruderalis, and the like), and any variants, hybrids, derivatives or combinations thereof. Hemp oils and cannabinoids can do a number of things like reduce risks of illnesses like Alzheimer's disease, cardiovascular disease, skin issues, stress, inflammation in the body, and the like. Other benefits of hemp oils and cannabinoids include, but are not limited to, lowering blood pressure, preventing relapse in drug and alcohol addiction, treating anxiety disorders, treating gastrointestinal disorders, preventing seizures, fighting cancer, and the like.

Material and thus particle size variations can yield different textures by simply adding to the base dispersion, for example, texturizing modifiers, like wax, stearic acid, and stearyl alcohols.

### Stability

It is the small size of the dispersion particles (i.e., dispersions of particles of one or more hydrophobic agents having an average particle size of about 5 m or less), together with a sufficient amount of polymer or copolymer fragments, or combinations thereof, that impart stability. From Stokes' Law, the small size minimizes the rate of sedimentation or creaming of the hydrophobic particles. Further, the uniform distribution or disperstivity of the particles minimizes the tendency of the hydrophobic particles to coalesce as would be predicted by the Ostwald Ripening equation. Still further, the presence of polymer or copolymer fragment provides a matrix or organized fluid that reduces Newtonian flow thereby reducing the number of particle collisions and potential coalescence. The commercially viable stability described above (30 days or more) allows a useful amount of time in which to store compositions (e.g., topical compositions) to maintain product integrity.

For determining a matrix, viscosity is measured in terms of a ratio of shearing stress to the velocity gradient in a fluid. If a sphere of known density is dropped into a fluid, the viscosity can be determined using the following formula:

$$η = 2 a {g}^{2} ( Δρ ) / 9 v$$

where Δρ is the density difference between fluid and sphere tested, a is the radius of the sphere, g is the acceleration due to gravity and v is the velocity of the sphere. To determine a matrix, a comparison can be made of the polymer and copolymer non-fluidized first dispersion, and the polymer and copolymer fragment fluidized second dispersion, against the aqueous solute fluid.

In pre and post microfluidization, a matrix will be established. The polymer and polymer fragments will achieve their viscosity when neutralized to 1-2 pH units above the pKa. Most cases this will be between pH 6 to pH 7. Even though the polymer system without fluidization will be thicker than the polymer and copolymer fragment system with fluidization, both will have a velocity greater than the system with no polymer. Thus, referring to the above formula, a different number greater than 1 will be obtained indicating a matrix has been formed. The surprising element will be that, even though the matrix has been reduced, the fluidized polymer and copolymer fragments, along with the small, uniform, and negative hydrophobic particle charge, keeps the system stable.

In determining a matrix, the very small particles produced by the ultra high shear and the polymer and copolymer fragments resulting from the process of this disclosure have tremendous surface area (referring to both volume of particles and increased volume of polymer/copolymer fragments). The volume relevant of each of these constituents (particles of hydrophobe and fragments) creates a matrix that is sufficient to support the weight or mass of the individual hydrophobic particles moving around the fluid, which then are affected by Stokes' Law, Ostwald Ripening, and Zeta Potential. All of these elements within the final matrix allow the hydrophobic particles and polymer and copolymer fragments to be stable and live together without separation for a long period of time.

In an embodiment, the compositions of this disclosure can be for a subphase of a 35% concentrated hydrophobic (or blend of hydrophobic) agent(s) dispersion that is then diluted in the finished good bulk. That dispersion may be used in the final finished goods bulk at a 1% level. Therefore, the hydrophobe is at 0.3%.

The particles of one or more hydrophobic agent(s) of this disclosure have an average particle size from about 100 nm to about 5 m, and are 85% or more of a size within ±2.0 standard deviations, or within ±1.9 standard deviations, or within ±1.8 standard deviations, or within ±1.7 standard deviations, or within ±1.6 standard deviations, or within ±1.5 standard deviations, of the average particle size.

The one or more polymer or copolymer fragments, or combinations thereof, are sufficient to stabilize the particles of one or more hydrophobic agent(s) in the dispersions, at a level from about 0.05% w/w to about 70% w/w, or from about 0.1% w/w to about 65% w/w, or from about 0.15% w/w to about 60% w/w, or from about 0.2% w/w to about 55% w/w, or from about 0.25% w/w to about 50% w/w, or from about 0.3% w/w to about 45% w/w, in the dispersions. Weight/weight equivalents can be calculated as known in the art.

The stability is further manifested in that two or more distinct dispersions can be mixed without decreasing the stability of the various component hydrophobic agent particles, or a dispersion can be diluted into aqueous fluid or aqueous-solute fluid without decreasing the stability of the component hydrophobic agent particles.

Further, if hydrophobic agent A were not compatible with hydrophobic agent B when mixed, nonetheless a dispersion of the disclosure of hydrophobic agent A can be mixed with a dispersion of hydrophobic B, since the individual particles maintain their integrity. Silicone Oil and olive oil exemplify such incompatible hydrophobic agents.

### Utility

The dispersions of this disclosure are useful in nutritional, pharmaceutical, biomedical, cosmetic, food, animal care, household, pet care, veterinary health, and other applications.

In the methods of the disclosure, animals treated can include, without limitation, humans, domesticated animals(such as dogs, cats, hamsters, gerbils, guinea pigs, cattle, pigs, sheep, goats, horses, zebus, donkeys, mules, buffalos, camels, yaks, mice, rats, other rodents, gayals, rabbits, alpacas, vicunas, llamas, poultry, other domesticated birds, and the like), wild animals, and the like.

A “cosmetic” material according to the disclosure is one that is generally recognized as safe for application to improve the appearance or odor of human or animal skin or mucosa. A “dermatologically appropriate material” is one that is generally recognized as safe for application to human or animal skin or mucosa. In embodiments, all the materials of a composition containing a dispersion of hydrophobic agents are dermatologically appropriate materials.

### Hand Sanitizer

In certain embodiments of the disclosure formulated as hand sanitizers, the composition can include for example:

| | Embodiment A | Embodiment B |
|---|---|---|
| | (by wt. %) | (by wt. %) |
| Ethanol or isopropyl alcohol | 50-89.99 | 50-89.99% wt |
| Water miscible solvent(s) of | [1-25] | [2-15] |
| Formula A that that are not | | |
| ethanol or isopropyl alcohol | | |
| Hydrophobic dispersions | [−20] | [5-15] |
| (5 μm or less average particle | | |
| size) of polymer or | | |
| copolymer fragments, or | | |
| combinations thereof, that are | | |
| skin conditioning or | | |
| modifying agents | | |
| Antioxidant | [0-0.2] | [0.02-0.1] |
| Thickener (rheological agent) | [0-3] | [0.1-1.75] |
| Hydrophobic dispersions | [0.01-10] | [1-7.5] |
| (5 μm or less average particle | | |
| size) of polymer or | | |
| copolymer fragments, or | | |
| combinations thereof, that are | | |
| therapeutic or functional | | |
| agents | | |
| Hydrophobic dispersions | [1-20] | [5-15] |
| (5 μm or less average particle | | |
| size) of polymer or | | |
| copolymer fragments, or | | |
| combinations thereof, that are | | |
| aesthetic modifying agents | | |
| Water | [5-40] | [10-30] |
| Neutralizing agent | [0-7.5] | [0.2-5] |

### Body Lotion

In certain embodiments of the disclosure formulated as body lotions, the composition can include for example:

| | Embodiment A | Embodiment B |
|---|---|---|
| | (by wt. %) | (by wt. %) |
| Hydrophobic dispersions | | |
| (5 μm or less average particle | | |
| size) of polymer or | | |
| copolymer fragments, or | | |
| combinations thereof, that are | | |
| aesthetic modifying agents | | |
| Water miscible solvent | [1-40] | [1-25] |
| Thickener (rheological agent) | [0-7.5] | [0.2-5] |
| Soothing Agent | [0-0.8] | [0.1-0.5] |
| Hydrophobic dispersions | [2-20] | [8-15] |
| (5 μm or less average particle | | |
| size) of polymer or | | |
| copolymer fragments, or | | |
| combinations thereof, that are | | |
| skin conditioning agents | | |
| Water | [20-90] | [30-70] |
| Neutralizing agent | [0-7.5] | [0.2-5] |

In an embodiment, the dispersion compositions of this disclosure can be formulated as a sunscreen. The dispersion compositions can contain active sunscreen agents, for example, ethylhexyl methoxycinnamate, avobenzone, benzophenones, octocrylene, homosalate, ethylhexyl salicylate, homomenthyl salicylate, triethanolamine salicylate, menthyl anthranilate, PABA, octyl dimethyl PABA, 2-ethoxyethyl p-methoxycinnamate, phenylbenzimidazole sulfonic acid, titanium dioxide, zinc oxide, and any derivatives or combinations thereof. The active sunscreen agents and other additives are present in the dispersion compositions in an amount from about 1 wt % to about 70 wt %, or from about 2 wt % to about 65 wt %, or from about 3 wt % to about 60 wt %, or from about 4 wt % to about 55 wt %, based on the total weight of the dispersion composition. The sunscreen formulation has a viscosity from about 1 cps to about 150,000 cps, or from about 2 cps to about 125,000 cps, or from about 3 cps to about 100,000 cps, or from about 4 cps to about 75,000 cps.

In an embodiment, the dispersion compositions of this disclosure can be formulated as a toner or toner/sunscreen. The dispersion compositions can contain toner ingredients, for example, witch hazel, alpha-hydroxy acids such as glycolic acid, lactic acid, and mandelic acid; beta-hydroxy acids such as salicylic acid; glycerin, hyaluronic acid, panthenol, niacinamide, allantoin, citric acid, hydrogenated starch hydrolysate, rosewater, orange flower water, tocopherol (vitamin E), vitamin C, vitamin CG, and the like. The toner formulation can also include active sunscreen agents as described above. The toner ingredients and other additives are present in the dispersion compositions in an amount from about 0.01 wt % to about 90 wt %, or from about 0.05 wt % to about 80 wt %, or from about 0.1 wt % to about 70 wt %, or from about 0.15 wt % to about 65 wt %. The toner formulation has a viscosity from about 1 cps to about 2,500 cps, or from about 2 cps to about 2,250 cps, or from about 3 cps to about 2,000 cps, or from about 4 cps to about 1,500 cps.

Other additives include, for example, antioxidants, buffering agents (to control pH); therapeutic agents including, for example, humectants, exfoliating agents, skin lightening agents, anti-wrinkle actives, anti-atrophy actives, moisturizing agents, anti-cellulite agents, skin soothing agents; chelating agents, neutralizing agents, freezing point lowering agents, odor control/fragrance, flavors, preservatives, rheological modifier, antifoams, and the like.

All ranges recited herein include ranges therebetween and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more.

The following are preferred embodiments of the compositions of this disclosure.

Embodiment 1. A composition comprising:
a dispersion comprising (i) particles of one or more hydrophobic agent(s) having an average particle size of about 5 m or less, (ii) one or more polymer or copolymer fragments, or combinations thereof, and (iii) an aqueous-solute fluid;
wherein the one or more polymer or copolymer fragments, or combinations thereof, are present in an amount sufficient to stabilize the particles of one or more hydrophobic agent(s) in said dispersion.

Embodiment 2. The composition of embodiment 1 wherein the particles of one or more hydrophobic agent(s) are present in an amount from about 0.01% wt. to about 70% wt., based on the total weight of the composition.

Embodiment 3. The composition of embodiment 1 wherein the aqueous-solute fluid is present in an amount from about 1.0% wt. to about 98.5% wt., based on the total weight of the composition.

Embodiment 4. The composition of embodiment 1 wherein the one or more polymer or copolymer fragments, or combinations thereof, are present in an amount from about 0.01% wt. to about 10% wt., based on the total weight of the composition.

Embodiment 5. The composition of embodiment 1 wherein the particles of one or more hydrophobic agent(s) (i) have an average particle size from 100 nm to 5 m, (ii) have a solubility of less than about 0.1% by weight in water under standard conditions, and (iii) are 85% or more of a size within ±2.0 standard deviations, or within ±1.9 standard deviations, or within ±1.8 standard deviations, or within ±1.7 standard deviations, or within ±1.6 standard deviations, or within ±1.5 standard deviations, of the average particle size.

Embodiment 6. The composition of embodiment 1 wherein the particles of one or more hydrophobic agent(s) (i) have an average particle size from about 100 nm to about 5 m, (ii) have a solubility of less than about 0.1% by weight in water under standard conditions, and (iii) are 90% or more of a size within ±2.0 standard deviations, or within ±1.9 standard deviations, or within ±1.8 standard deviations, or within ±1.7 standard deviations, or within ±1.6 standard deviations, or within ±1.5 standard deviations, of the average particle size.

Embodiment 7. The composition of embodiment 1 wherein the one or more polymer or copolymer fragments, or combinations thereof, are sufficient to stabilize the particles of one or more hydrophobic agent(s) in said dispersion, at a level from about 0.05% w/w to about 70% w/w of said one or more hydrophobic agent(s), in said dispersion.

Embodiment 8. The composition of embodiment 1, which is prepared by a process comprising:
preparing a premix comprising (i) one or more hydrophobic agent(s), (ii) one or more polymers, one or more copolymers, or combinations thereof, (iii) an aqueous-solute fluid, and optionally (iv) one or more additive(s);
subjecting the premix to low energy mixing to form a first dispersion; and
subjecting the first dispersion to ultra-high energy mixing to form a second dispersion.

Embodiment 9. The composition of embodiment 8, wherein the low energy mixing is mechanical and performed at ambient temperature and pressure.

Embodiment 10. The composition of embodiment 8, wherein the low energy mixing is performed using a homogenizer, rotor stator, vacuum homogenizer, media mill, colloid mill, or combinations thereof.

Embodiment 11. The composition of embodiment 8, wherein the first dispersion has an average particle size of one or more hydrophobic agent(s) of less than 200 microns, or less than 150 microns, or less than 100 microns.

Embodiment 12. The composition of embodiment 8, wherein the ultra-high energy mixing is performed using a high-pressure high-shear device, sonicator, or combinations thereof.

Embodiment 13. The composition of embodiment 8 wherein the ultra-high energy mixing is non-mechanical and performed at a temperature from about 15° C. to about 30° C. and until about 85 wt. % of the total hydrophobic particles of one or more hydrophobic agent(s) in the second dispersion are within ±2.0 standard deviations, or within ±1.9 standard deviations, or within ±1.8 standard deviations, or within ±1.7 standard deviations, or within ±1.6 standard deviations, or within ±1.5 standard deviations, of the average particle size of the second dispersion, the average particle size of the one or more hydrophobic agent(s) in the second dispersion being from about 100 nm to about 5 μm.

Embodiment 14. The composition of embodiment 8, wherein the ultra-high energy mixing is performed at a temperature from about 20° C. to about 25° C.

Embodiment 15. The composition of embodiment 8, wherein the ultra-high energy mixing is performed until 85 wt. % of the total hydrophobic particles in the dispersion are within ±2.0 standard deviations, or within ±1.9 standard deviations, or within ±1.8 standard deviations, or within ±1.7 standard deviations, or within ±1.6 standard deviations, or within ±1.5 standard deviations, of the average particle size of the second dispersion.

Embodiment 16. The composition of embodiment 8, wherein 90 wt. % of the total hydrophobic particles in the second dispersion are within ±2.0 standard deviations, or within ±1.9 standard deviations, or within ±1.8 standard deviations, or within ±1.7 standard deviations, or within ±1.6 standard deviations, or within ±1.5 standard deviations, of the average particle size of the second dispersion.

Embodiment 17. The composition of embodiment 8, wherein the average particle size of the second dispersion is from about 150 nm to about 5 m.

Embodiment 18. The composition of embodiment 8, wherein the average particle size of the second dispersion is from about 170 nm to about 2.5 m.

Embodiment 19. The composition of embodiment 8, wherein the ultra-high energy mixing is performed until 90 wt. % of the total hydrophobic particles in the second dispersion are within ±2.0 standard deviations, or within ±1.9 standard deviations, or within ±1.8 standard deviations, or within ±1.7 standard deviations, or within ±1.6 standard deviations, or within ±1.5 standard deviations, of the average particle size of the second dispersion.

Embodiment 20. The composition of embodiment 8, wherein the ultra-high energy mixing is performed until 93 wt. % of the total hydrophobic particles in the second dispersion are within ±2.0 standard deviations, or within ±1.9 standard deviations, or within ±1.8 standard deviations, or within ±1.7 standard deviations, or within ±1.6 standard deviations, or within ±1.5 standard deviations, of the average particle size of the second dispersion.

Embodiment 21. The composition of embodiment 8, wherein the ultra-high energy mixing is performed until 95 wt. % of the total hydrophobic particles in the second dispersion are within ±2.0 standard deviations, or within ±1.9 standard deviations, or within ±1.8 standard deviations, or within ±1.7 standard deviations, or within ±1.6 standard deviations, or within ±1.5 standard deviations, of the average particle size of the second dispersion.

Embodiment 22. The composition of embodiment 8, wherein the ultra-high energy mixing is performed until 95 wt. % of the total hydrophobic particles in the second dispersion are within ±2.0 standard deviations, or within ±1.75 standard deviations, or within ±1.5 standard deviations, of the average particle size of the second dispersion.

Embodiment 23. The composition of embodiment 8, wherein after ultra-high energy mixing, the average particle size of the one or more hydrophobic agent(s) in the second dispersion ranges from about 100 nm to about 2.5 m, or from about 125 nm to about 900 nm, or from about 150 nm to about 800 nm, or from about 175 nm to about 750 nm, or from about 200 nm to about 700 nm, or from about 250 nm to about 600 nm.

Embodiment 24. The composition of embodiment 8, wherein the one or more polymer or copolymer fragments, or combinations thereof, of the composition exhibit at least a 10%, or 20%, or 30%, or 40%, or 50%, or 60%, or 70%, or 80%, or 90%, reduction in viscosity, as compared to the viscosity of the same one or more polymers, copolymers, or combinations thereof, of the premix, under standard conditions.

Embodiment 25. The composition of embodiment 1, wherein the one or more polymer or copolymer fragments, or combinations thereof, have a sufficient water dispersibility, alone or when modified by an organic or inorganic substituent, to modify the rheological properties of the aqueous-solute fluid.

Embodiment 26. The composition of embodiment 1, wherein the one or more polymer or copolymer fragments, or combinations thereof, include, but are not limited to: fragments of the following: acrylates, methacrylates, acrylamides, vinyls, polyethylene and derivatives thereof, and any derivatives or combinations thereof.

Embodiment 27. The composition of embodiment 1, wherein the one or more polymer or copolymer fragments, or combinations thereof, include, but are not limited to: fragments of the following: carbomer and derivatives and salts thereof, acrylates/C10-C30 alkyl acrylate crosspolymer, acrylates/ceteth-20 itaconate copolymer, acrylates/ceteth-methacrylate copolymers, acrylates/steareth-20 methacrylate copolymers, acrylates/steareth-20 itaconate copolymers, acrylates/steareth-50 acrylate copolymers, acrylates/VA crosspolymers, acrylates/vinyl isodecanoate crosspolymers, acrylic acid/acrylonitrogen copolymers, ammonium acrylates/acrylonitrogen copolymers, glyceryl polymethacrylate, polyacrylic acid, PVM/MA decadiene crosspolymer, sodium acrylate/vinyl isodecanoate crosspolymers, sodium carbomer, ethylene/acrylic acid copolymer, ethylene/VA copolymer, acrylates/acrylamide copolymer, acrylate copolymers, acrylates/hydroxyester acrylate copolymers, acrylate/octylarylamide copolymers, acrylates/PVP copolymers, AMP/acrylate copolymers, butylester of PVM-MA copolymer, carboxylate vinyl acetate terpolymers, diglycol/CHDM/isophthalates/SIP copolymer, ethyl ester of PVM-MA copolymer, isopropyl ester of PVM-MA copolymer, octylacrylamide/acrylate/butylaminoethyl methacrylate copolymers, polymethacrylamidopropyltrimonium chloride, polyvinylcaprolactam, PVP, PVP/dimethylaminoethylmethacrylate copolymer, PVP/DMAPA acrylate copolymers, PVP/carbamyl polyglycol ester, PVP/VA copolymer, PVP/VA vinyl propionate copolymer, PVP/vinylcaprolactam/DMAPA acrylate copolymers, sodium polyacrylate, VA/butyl maleate/isobornyl acrylate copolymers, VA/crotonates copolymer, VA/crotonates vinyl neodecanoate copolymer, VA/crotonates/vinyl propionate copolymer, vinyl caprolactam/PVP/dimethylaminoethylmethacrylate copolymer, hydroxyethyl acrylate/copolymer, sodium acrylate/sodium acryloyldimethyl taurate copolymer (and) C15-C19 alkane (and) polyglyceryl-6 laurate (and) polyglycerin-6, polyquarternium-37, ammonium acryloyldimethyltaurate/VP copolymer, hydroxyethyl acrylate/sodium acryloyldimethyl taurate copolymer, polyacrylate crosspolymer-6, and any derivatives or combinations thereof.

Embodiment 28. The composition of embodiment 1 wherein the one or more polymer or copolymer fragments, or combinations thereof, are present in an amount from about 0.05% wt. to about 5% wt., or from about 0.1% wt. to about 4% wt., or from about 0.2% wt. to about 3% wt., based on the total weight of the composition.

Embodiment 29. The composition of embodiment 1, wherein the one or more hydrophobic agent(s) include, but are not limited to: mono, di, tri, or poly alkyl (or alkenyl) esters, ethers, acetals and hemiacetals of a di-, tri-, or polyhydroxy compound, and any derivatives or combinations thereof; saturated and unsaturated, linear and branched natural or vegetable oils including, but not limited to soybean oil, almond oil, castor oil, canola oil, cottonseed oil, grapeseed oil, rice bran oil, palm oil, coconut oil, palm kernel oil, olive oil, linseed oil, sunflower oil, safflower oil, peanut oil, corn oil, and any derivatives or combinations thereof; water insoluble silicone materials including, but not limited to polyalkylsiloxanes, polyarylsiloxanes, polyalkylarylsiloxanes, polysiloxane gums, polyethersiloxane copolymers, silicone crosspolymers, and any derivatives or combinations thereof; and water insoluble materials including, but not limited to diglycerides, triglycerides, lauramine oleate, isopropyl palmitate, mineral oil, petrolatum, and any derivatives or combinations thereof.

Embodiment 30. The composition of embodiment 1, wherein the one or more hydrophobic agent(s) comprise one or more therapeutic agent(s).

Embodiment 31. The composition of embodiment 8, further comprising: adding one or more functional agent(s) to the premix, or post-treating the first dispersion with one or more functional agent(s), or post-treating the second dispersion with one or more functional agent(s).

Embodiment 32. The composition of embodiment 30, wherein the one or more therapeutic agent(s) comprise one or more hydrophobic agent(s) or one or more hydrophilic agent(s), and include, but are not limited to: anti-acne agents, antimicrobial agents, anti-inflammatory agents, analgesics, anti-erythemal agents, anti-pruritic agents, anti-edemal agents, anti-psoriatic agents, anti-fungal agents, skin protectants, sunscreen agents, vitamins, antioxidants, scavengers, anti-irritants, anti-bacterial agents, antiviral agents, antiaging agents, photoprotection agents, hair growth enhancers, hair growth inhibitors, hair removal agents, antidandruff agents, anti-seborrheic agents, exfoliating agents, wound healing agents, anti-ectoparasitic agents, sebum modulators, immunomodulators, hormones, botanicals, moisturizing agents, hand sanitizing agents, astringents, sensates, antibiotics, anesthetics, steroids, tissue healing substances, tissue regenerating substances, amino acids, peptides, minerals, ceramides, biohyaluronic acids, bleaching ingredients, and any derivatives or combinations thereof.

Embodiment 33. The composition of embodiment 30, wherein the one or more therapeutic agent(s) comprise at least one sunscreen agent including, but not limited to ethylhexyl methoxycinnamate, avobenzone, benzophenones, octocrylene, homosalate, ethylhexyl salicylate, homomenthyl salicylate, triethanolamine salicylate, menthyl anthranilate, PABA, octyl dimethyl PABA, 2-ethoxyethyl p-methoxycinnamate, phenylbenzimidazole sulfonic acid, titanium dioxide, zinc oxide, and any derivatives or combinations thereof.

Embodiment 34. The composition of embodiment 1, wherein the one or more hydrophobic agent(s) comprise at least one aesthetic modifying agent.

Embodiment 35. The composition of embodiment 8, further comprising treating or post-treating the premix, or post-treating the first dispersion, or post-treating the second dispersion, such that: pH is raised or lowered by the addition of an alkali or acid, respectively; viscosity is increased or decreased by the addition of a thickening agent or salt, respectively; specific gravity is adjusted by the addition of one or more of an antifoam, centrifugation, vacuum, and reduction in viscosity with sweeping mixing; refractive index is adjusted up or down by the addition of a high refractive index solvent/solute or water; or active levels are adjusted by the addition of a hydrophobic active dispersion.

Embodiment 36. The composition of embodiment 1, wherein the one or more hydrophobic agent(s) comprise at least one compound including, but not limited to:
(b) a compound having the formula A

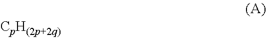

wherein p is a positive integer greater than or equal to 6 and q is 0 or a positive, even integer no greater than p;
(b) a compound having the formula B or C

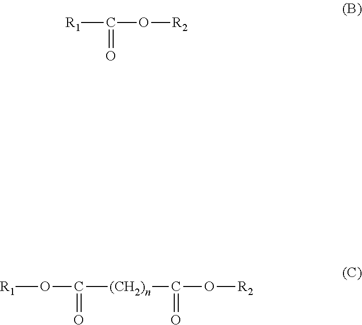

wherein R1 is a saturated or unsaturated, linear, branched or cyclic C₁-C₂₃ acyl moiety having 0, 1, or more substituent groups; and R₂ is hydrogen or a saturated or unsaturated, linear, branched or cyclic C₁-C₂₄ acyl moiety having 0, 1, or more substituent groups, and n is an integer from 0 to 20;
(c) a compound having the formula D

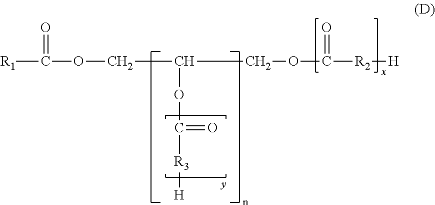

wherein R1 is a C₆ to C₂₄ acyloate group comprising saturated, unsaturated, cyclic, branched, substituted, oxidized, reduced, polymeric, or copolymeric hydrocarbon(s); R2 and R3 are independently a C₃ to C₂₄ acyloate group comprising saturated, unsaturated, cyclic, branched, substituted, oxidized, reduced, polymeric, or copolymeric hydrocarbon(s), less 1 hydrogen at the omega carbon; x is a value of 0 or 1; y is a value of 0 to n; and n is a value of 1 to 6; and
(d) a silicone or modified polysiloxane.

Embodiment 37. The composition of embodiment 34, wherein the one or more aesthetic modifying agent(s) comprise at least one compound including, but not limited to: polysiloxanes, cyclic siloxanes, polyalkylsiloxanes, polyarylsiloxanes, polyalkylarylsiloxanes, polysiloxane gums, polyethersiloxane copolymers, and silicone crosspolymers.

Embodiment 38. The composition of embodiment 34, wherein the one or more aesthetic modifying agent(s) comprise one or more of C₂-C₂6 alkanes substituted with 2-24 hydroxyls, wherein the hydroxyls of the foregoing compounds are independently acylated with a saturated, unsaturated, linear, branched or cyclic C₁-C₂₄ alkane, rendering the substituted alkyls hydrophobic agents.

Embodiment 39. The composition of embodiment 1, wherein the one or more hydrophobic agent(s) comprise colorants including, but not limited to: annatto oil, paprika oil, chlorophyll, lycopene, carotenoids, xanthophylls, and any derivatives or combinations thereof.

Embodiment 40. The composition of embodiment 1, wherein the one or more hydrophobic agent(s) comprise essential nutrients including, but not limited to: vitamins such as vitamin D) and its derivatives, vitamin A and its derivatives, vitamin E and its derivatives, vitamin K, vitamin F, vitamin P, lipoic acid, lycopene, phospholipids, ceramides, ubiqinone, sterols, flavonoids, cholesterol, sphingolipids, prostaglandins, docosahexaenoic acid, and any derivatives or combinations thereof.

Embodiment 41. The composition of embodiment 1, wherein the one or more hydrophobic agent(s) comprise fragrances or flavors including, but not limited to: terpenes, isoterpenenes, alkyl lactones, essential oils, natural oils, vanilla, and any derivatives or combinations thereof, Embodiment 42. The composition of embodiment 1, wherein the one or more hydrophobic agent(s) comprise one or more therapeutic agent(s) and at least one aesthetic modifying agent.

Embodiment 43. The composition of embodiment 31, wherein the one or more functional agent(s) include, but are not limited to surfactants, neutralizing agents, chelating agents, foaming agents, rheological modifying agents, sensates, and any derivatives or combinations thereof.

Embodiment 44. The composition of embodiment 1, wherein the one or more hydrophobic agent(s) are present in an amount of 0.05% wt, to 70% wt, or 0,1% wt. to 60% wi, or 0.5% wt. to 55% wt., or 1% wt to 50% w., based on the total weight of the composition.

Embodiment 45. The composition of embodiment 8, wherein the particles of hydrophobic agent(s), after low energy mixing, have an average particle size in the range between about 5 microns to about 100 microns, or between about 5 microns to about 50 microns.

Embodiment 46. The composition of embodiment 8, wherein the particles of hydrophobic agent(s), after ultra-high energy mixing, have an average particle size in the range between about 125 nm to about 5 mμ or between about 150 nm to about 2.5 m.

Embodiment 47. The composition of embodiment 1, wherein the aqueous-solute fluid comprises water or a combination of water and one or more polar solute(s).

Embodiment 48. The composition of embodiment 47, wherein the one or more polar solutes have a water solubility of 0.1% or greater at a temperature of 23° C. and a pressure of 100 kPa (1 bar).

Embodiment 49. The composition of embodiment 47, wherein the one or more polar solutes have a dielectric constant of greater than 10 at a temperature of 23° C. and a pressure of 100 kPa (1 bar).

Embodiment 50. The composition of embodiment 47, wherein the one or more polar solutes include, but are not limited to: water soluble solids at a temperature of 23° C. and a pressure of 100 kPa (1 bar), water soluble liquids at a temperature of 23° C. and a pressure of 100 kPa (1 bar), and any derivatives or combinations thereof.

Embodiment 51. The composition of embodiment 50, wherein the water soluble solids include, but are not limited to: carbohydrates; amino acids, peptides, and proteins; vitamins; minerals; and any derivatives or combinations thereof.

Embodiment 52. The composition of embodiment 51, wherein the carbohydrates include, but not limited to: monosaccharides, reduced sugar alcohols, sugar acids, substituted monosaccharides, disaccharides, triglycerides, and polysaccharides (glycans), and any derivatives or combinations thereof.

Embodiment 53. The composition of embodiment 52, wherein the monosaccharides include, but are not limited to: 1, 3-dihydroxy-2-propanone, arabinose, ribose, xylose, lyxose, ribulose, xylulose, psicose, sorbose, tagatose, threose, erythrulose, glucose, fructose, mannose, galactose, allose, altrose, gulose, indose, talose, dihydroxacetone, and any derivatives or combinations thereof.

Embodiment 54. The composition of embodiment 52, wherein the reduced sugar alcohols include, but are not limited to: threitol, arabitol, xylitol, ribitol, mannitol, sorbitol, galactitol, fucitol, iditol, inositol, volemitol, isomalt, maltitol, lactitol, and any derivatives or combinations thereof.

Embodiment 55. The composition of embodiment 52, wherein the sugar acids include, but are not limited to: aldonic acids including glyceric acid, xylonic acid, gluconic acid, threonic acid, and ascorbic acid; ulosonic acids including neuraminic acid (5-amino-3,5-dideoxy-D-glycero-D-galacto-non-2-ulosonic acid), and ketodeoxyoctulosonic acid (KDO or 3-deoxy-D-manno-oct-2-ulosonic acid); uronic acids including glucuronic acid, galacturonic acid, lactobionic acid, and iduronic acid; and aldaric acids including tartaric acid, meso-galactaric acid (mucic acid), and D-glucaric acid (saccharic acid); and any derivatives or combinations thereof.

Embodiment 56. The composition of embodiment 52, wherein the substituted monosaccharides include, but are not limited to sugar esters including phosphate sugar esters, amino sugar esters, acylate sugar esters, and any derivatives or combinations thereof.

Embodiment 57. The composition of embodiment 56, wherein the phosphate sugar esters include, but are not limited to glucose-1-phosphate, fructose-1,6-diphosphate, and any derivatives or combinations thereof.

Embodiment 58. The composition of embodiment 56, wherein the amino sugar esters include, but are not limited to 2-glucosamine, 2-galactosamine, N-acetylglucosamine, N-acetylmannosamine, neuraminic acid, N-acetyltalosaminuronic acid, and any derivatives or combinations thereof.

Embodiment 59. The composition of embodiment 56, wherein the acylate sugar esters include, but are not limited to methyl-glucoside, muramic acid, N-acetyl-neuraminic acid, N-glycosyl-neuraminic acid, pangamic acid, and any derivatives or combinations thereof.

Embodiment 60. The composition of embodiment 52, wherein the disaccharides include, but are not limited to sucrose (fructose-glucose), lactose (galactose-glucose), maltose (glucose-glucose), isomaltose, maltobiose, trehalose, cellobiose, and any derivatives or combinations thereof.

Embodiment 61. The composition of embodiment 52, wherein the triglycerides include, but are not limited to raffinose (glucose-fructose-galactose), melizitose, and any derivatives or combinations thereof.

Embodiment 62. The composition of embodiment 52, wherein the polysaccharides (glycans) include, but are not limited to starch, glycogen, amylopectin, amylose, cellulose, dextran, chitan, alginic acid, agarose, glycosylaminoglycans including chondroitin sulfate, heparin, hyaluronic acid, dermatan sulfate, keratan sulfate, ascorbic acid (vitamin C), the 3-D form of glucuronic acid, and any derivatives or combinations thereof.

Embodiment 63. The composition of embodiment 51, wherein the amino acids, peptides, and proteins include, but are not limited to: alpha-amino acids including alanine, arginine, asparagine, aspartate, cysteine, glutamate, glutamine, glycine, histidine, isoleucine, leucine, lysine, methionine, phenylalanine, proline, serine, threonine, tryptophan, valine, selenocysteine, pipecolic acid, pyrrolysine, and any derivatives or combinations thereof; beta-amino acids including beta-alanine, beta-aminoisobutyric acid, and any derivatives or combinations thereof; gamma-amino acids including gamma-amino butyric acid, carnitine, and any derivatives or combinations thereof; dipeptides including cysteinyl-thionine, glycyl-glycine, alanyl-histidine, cysteinyl-glycine, and any derivatives or combinations thereof; tripeptides including glutathione, glycyl-glycyl-glycine, lysyl-lysyl-lysine, glutamyl-histeinyl-glycine, and any derivatives or combinations thereof; substituted amino acids and peptides including acetyl carnitine, acetyl cysteine, methylglycinate, glutathione methyl ester, and any derivatives or combinations thereof; proteins including enzymes, cytokines, growth factors, structural proteins such as collagen, elastin, keratin, and any derivatives or combinations thereof.

Embodiment 64. The composition of embodiment 51, wherein the vitamins include, but are not limited to: vitamin C (ascorbic acid or water soluble derivatives of ascorbic acid), vitamin B1 (thiamin), vitamin B2 (riboflavin), vitamin B3 (niacin), vitamin B5 (pantothenic acid), vitamin B6 (pyridoxin), vitamin B7 (biotin), vitamin B9 (folate), vitamin B12 (cobalamin), and any derivatives or combinations thereof.

Embodiment 65. The composition of embodiment 51, wherein the minerals include, but are not limited to: aluminum bromide, aluminum chlorate, aluminum chloride, aluminum nitrate, aluminum sulfate, ammonium acetate, ammonium bromide, ammonium carbonate, ammonium chlorate, ammonium chloride, ammonium fluoride, ammonium hydrogen carbonate, ammonium iodide, ammonium nitrate, ammonium phosphate, ammonium sulfate, ammonium sulfide, ammonium sulfite, barium acetate, barium bromide, barium chlorate, barium chloride, barium hydroxide, barium iodide, barium nitrate, barium nitrite, calcium acetate, calcium bromide, calcium chlorate, calcium chloride, calcium iodide, calcium nitrate, calcium nitrite, cobalt (III) acetate, cobalt (III) bromide, cobalt (III) chlorate, cobalt (III) chloride, cobalt (III) iodide, cobalt (III) nitrate, cobalt (III) sulfate, copper (II) acetate, copper (II) bromide, copper (II) chlorate, copper (II) chloride, copper (II) fluoride, copper (II) nitrate, copper (II) sulfate, iron (II) acetate, iron (II) bromide, iron (II) chloride, iron (II) iodide, iron (II) nitrate, iron (II) sulfate, iron (III) bromide, iron (III) chloride, iron (III) iodide, iron (III) nitrate, iron (III) sulfate, lead (II) acetate, lead (II) chlorate, lead (II) nitrate, lead (II) nitrite, lithium acetate, lithium bromide, lithium carbonate, lithium chlorate, lithium chloride, lithium fluoride, lithium hydrogen carbonate, lithium hydroxide, lithium iodide, lithium nitrate, lithium nitrite, lithium sulfate, lithium sulfide, lithium sulfite, magnesium acetate, magnesium bromide, magnesium chlorate, magnesium chloride, magnesium iodide, magnesium nitrate, magnesium nitrite, magnesium sulfate, magnesium sulfite, nickel acetate, nickel bromide, nickel chlorate, nickel chloride, nickel fluoride, nickel iodide, nickel nitrate, nickel sulfate, potassium acetate, potassium bromide, potassium carbonate, potassium chlorate, potassium chloride, potassium fluoride, potassium hydrogen carbonate, potassium hydroxide, potassium iodide, potassium nitrate, potassium nitrite, potassium phosphate, potassium sulfate, potassium sulfide, potassium sulfite, silver chlorate, silver fluoride, silver nitrate, sodium acetate, sodium bromide, sodium carbonate, sodium chlorate, sodium chloride, sodium fluoride, sodium hydrogen carbonate, sodium hydroxide, sodium iodide, sodium nitrate, sodium nitrite, sodium phosphate, sodium sulfate, sodium sulfide, sodium sulfite, zinc acetate, zinc bromide, zinc chlorate, zinc chloride, zinc fluoride, zinc iodide, zinc nitrate, zinc sulfate, and any derivatives or combinations thereof.

Embodiment 66. The composition of embodiment 50, wherein the water soluble liquids comprise flowable, non-viscous, semi-viscous, or viscous liquids.

Embodiment 67. The composition of embodiment 66, wherein the water soluble liquids include, but are not limited to: glyceraldehyde, erythrose, erythrulose, sedoheptulose, and any derivatives or combinations thereof.

Embodiment 68. The composition of embodiment 66, wherein the water soluble liquids comprise water miscible liquids.

Embodiment 69. The composition of embodiment 68, wherein the water miscible liquids include, but are not limited to: acetaldehyde, acetic acid, acetone, 1,2-butylene glycol, 1,3-butylene glycol, 1,4-butylene glycol, 2-butoxyethanol, dimethyl sulfoxide, ethanol, ethoxydiglycol, triethylene glycol, ethylene glycol, methanol, isopropanol, 1,2-propylene glycol, 1,3-propylene glycol, 1-propanol, propanoic acid, diglycerin, polyglycerol, glycerin, 1.5-pentylene glycol, hexylene glycol, and any derivatives or combinations thereof.

Embodiment 70. The composition of embodiment 50, wherein at least 10%, or at least 15%, or at least 20%, or at least 25%, of the water soluble liquids comprise water miscible liquids.

Embodiment 71. The composition of embodiment 1, wherein the water, or combination of water and one or more polar solute(s), is present in an amount from about 5% wt. to about 98% wt., or from about 10% wt. to about 95% wt., or from about 20% wt. to about 95% wt., based on the total weight of the composition.

Embodiment 72. The composition of embodiment 8, wherein the one or more additive(s) comprise one or more active or therapeutic ingredient(s); wherein the one or more active or therapeutic ingredient(s) include, but are not limited to: anti-acne agents, antimicrobial agents, anti-inflammatory agents, analgesics, anti-erythemal agents, antipruritic agents, antiedemal agents, anti-psoriatic agents, antifungal agents, skin protectants, sunscreen agents, vitamins, antioxidants, anti-irritants, anti-bacterial agents, antiviral agents, antiaging agents, photoprotection agents, exfoliating agents, wound healing agents, sebum modulators, immunomodulators, hormones, botanicals, moisturizing agents, hand sanitizing agents, astringents, sensates, antibiotics, anesthetics, steroids, tissue healing substances, tissue regenerating substances, amino acids, peptides, minerals, ceramides, hyaluronic acids, skin bleaching ingredients, pre-biotics, probiotics, hemp oils, cannabinoids, and humectants.

Embodiment 73. The composition of embodiment 72, wherein the one or more additive(s) are present in an amount from about 0.005% wt. to about 90% wt., or from about 0.01% wt. to about 75% wt., based on the total weight of the composition.

Embodiment 74. The composition of embodiment 1, which has a viscosity from about 1 cps to about 200,000 cps.

Embodiment 75. The composition of embodiment 1, which has a viscosity from about 10 cps to about 200,000 cps.

Embodiment 76. The composition of embodiment 1, which has a pH from about 3 to about 9, or from about 3.5 to about 8.5, or from about 4 to about 8.

Embodiment 77. The composition of embodiment 8, wherein the one or more polymers, copolymers, or combinations thereof, of the premix have a molecular weight from about 5000 daltons to about 2 million daltons, or from about 10,000 daltons to about 1.5 million daltons, or from about 15,000 daltons to about 1 million daltons.

Embodiment 78. The composition of embodiment 8, which is post-treated to modify physical, visual, tactile, oral, or olfactory properties.

Embodiment 79. The composition of embodiment 78, wherein the post-treatment of the composition comprises: pH is raised or lowered by the addition of an alkali or acid, respectively; viscosity is increased or decreased by the addition of a thickening agent or salt, respectively; specific gravity is adjusted by the addition of one or more of an antifoam, centrifugation, vacuum, and reduction in viscosity with sweeping mixing; refractive index is adjusted up or down by the addition of a high refractive index solvent/solute or water; or active levels are adjusted by the addition of a hydrophobic active dispersion.

Embodiment 80. The composition of embodiment 1 for use in one or more of nutritional, pharmaceutical, biomedical, cosmetic, food, animal care, veterinary health, household, and pet care applications.

Embodiment 81. The composition of embodiment 1 for application to skin, hair or external mucosa, or other surfaces and substrates.

Embodiment 82. The composition of embodiment 1, which is in the form of, or formulated as, a light liquid, heavy liquid, gel, light or soft cream, heavy or rich cream, light lotion, viscous lotion, butter, balm, stick, spray, mist, light fluid, rich fluid, liquid/toner, fluid/serum, or foam.

Embodiment 83. The composition of embodiment 1, which is formulated as a sunscreen lotion, a sunscreen spray, a sunscreen toner, a moisturizing hand sanitizer, a body lotion, a body spray, a natural oil liquid, a natural oil spray, a natural oil toner, an exfoliating mask, a lubricating shaving gel, a cream, or cannabinoids and their derivatives.

Embodiment 84. A composition comprising the first dispersion of embodiment 8.

Embodiment 85. A composition comprising the second dispersion of embodiment 8.

Embodiment 86. The composition of embodiment 8 wherein the second dispersion is further diluted with additional aqueous-solute fluid to achieve preset or desired aesthetic and performance properties.

Embodiment 87. The composition of embodiment 8 which is a mixture of two or more second dispersions to achieve preset or desired aesthetic and performance properties.

Embodiment 88. The composition of embodiment 8 in which one or more of viscosity, pH, specific gravity, refractive index, and active levels in the second dispersion are adjusted to achieve preset or desired aesthetic and performance properties.

Embodiment 89. The composition of embodiment 8, further comprising adding to the second dispersion one or more rheological modifying agent(s) to adjust viscosity of the second dispersion.

Embodiment 90. The composition of embodiment 1, wherein the dispersion provides a multifunctional delivery vehicle for active or therapeutic ingredients to a human or animal, said active or therapeutic ingredients including, but not limited to: anti-acne agents, antimicrobial agents, anti-inflammatory agents, analgesics, anti-erythemal agents, antipruritic agents, antiedemal agents, anti-psoriatic agents, antifungal agents, skin protectants, sunscreen agents, vitamins, antioxidants, anti-irritants, anti-bacterial agents, antiviral agents, antiaging agents, photoprotection agents, exfoliating agents, wound healing agents, sebum modulators, immunomodulators, hormones, botanicals, moisturizing agents, hand sanitizing agents, astringents, sensates, antibiotics, anesthetics, steroids, tissue healing substances, tissue regenerating substances, amino acids, peptides, minerals, ceramides, hyaluronic acids, skin bleaching ingredients, pre-biotics, probiotics, hemp oils, cannabinoids, and any derivatives or combinations thereof.

Embodiment 91. The composition of embodiment 1, wherein the one or more hydrophobic agent(s) comprise edible hydrophobic agent(s).

Embodiment 92. The composition of embodiment 91, wherein the edible hydrophobic agent(s) include, but are not limited to canola oil, corn oil, cottonseed oil, flaxseed oil, grape seed oil, peanut oil, safflower oil, sesame oil, soybean oil, sunflower oil, walnut oil, olive oil, peppermint oil, orange oil, and any derivatives or mixtures thereof.

Embodiment 93. The composition of embodiment 1, wherein the one or more polymer or copolymer fragments, or combinations thereof comprise edible polymer or copolymer fragments, or combinations thereof.

Embodiment 94. The composition of embodiment 93, wherein the edible polymer or copolymer fragments, or combinations thereof, include, but are not limited to polysaccharide fragments, and protein fragments.

Embodiment 95. The composition of embodiment 93 wherein the edible polymer or copolymer fragments, or combinations thereof, include, but are not limited to fragments of: waxes such as beeswax, candelilla, carnauba wax; phospholipids, fatty acids, triglycerides, glycolipids, starch, pectin, agar, alginate, cellulose derivatives, carrageenan, chitosan, xanthan gum, guar gum, gum Arabic, pullulan, albumin, collagen, gelatin, milk, zein, wheat gluten, soy, peanut, pea, and nut proteins.

Embodiment 96. The composition of embodiment 8, wherein the one or more polymers or copolymers, or combinations thereof, in the premix, after undergoing ultra-high energy mixing, are transformed into the one or more polymer or copolymer fragments, or combinations thereof, in the second dispersion.

Embodiment 97. The composition of embodiment 1, wherein the polymer and copolymer fragments, and any derivatives or combinations thereof, include fragments of polymeric and copolymeric rheological modifying agents.

Embodiment 98. The composition of embodiment 8, wherein one or more rheological modifying agents are added to the premix before ultra-high energy mixing.

Embodiment 99. The composition of embodiment 98, wherein the one or more rheological modifying agents comprise one or more polymeric and copolymeric rheological modifying agents.

Embodiment 100. The composition of embodiment 8, wherein one or more rheological modifying agents are added to the second dispersion after ultra-high energy mixing.

Embodiment 101. The composition of embodiment 2 where a hydrophobic active or therapeutic agent, or combination of hydrophobic active or therapeutic agents, are first dissolved into a second hydrophobic agent, or combination of hydrophobic agents, prior to the premix phase.

Embodiment 102. A composition comprising:
a dispersion comprising (i) particles of one or more hydrophobic agent(s), (ii) one or more polymer or copolymer fragments, or combinations thereof, and (iii) an aqueous-solute fluid;
wherein the particles of one or more hydrophobic agent(s) are present in an amount from about 0.01% wt. to about 70% wt., the aqueous-solute fluid is present in an amount from about 1.0% wt. to about 98.5% wt., and the one or more polymer or copolymer fragments, or combinations thereof are present in an amount from about 0.01% wt. to about 10% wt., all based on the total weight of the composition;
wherein the particles of one or more hydrophobic agent(s) (i) have an average particle size from about 100 nm to about 5 m, (ii) have a solubility of less than about 0.1% by weight in water under standard conditions, and (iii) are 85% or more of a size within ±2.0 standard deviations, or within ±1.9 standard deviations, or within ±1.8 standard deviations, or within ±1.7 standard deviations, or within ±1.6 standard deviations, or within ±1.5 standard deviations, of the average particle size; and
wherein the one or more polymer or copolymer fragments, or combinations thereof, are sufficient to stabilize the particles of one or more hydrophobic agent(s) in said dispersion, at a level from about 0.05% w/w to about 70% w/w of said one or more hydrophobic agent(s), in said dispersion.

Embodiment 103. A process comprising:
preparing a premix comprising (i) one or more hydrophobic agent(s), (ii) one or more polymers, one or more copolymers, or combinations thereof, (iii) an aqueous-solute fluid, and optionally (iv) one or more additive(s);
subjecting the premix to low energy mixing to form a first dispersion; and
subjecting the first dispersion to ultra-high energy mixing to form a second dispersion;
wherein said second dispersion comprises (i) particles of one or more hydrophobic agent(s) having an average particle size of about 5 m or less, (ii) one or more polymer or copolymer fragments, or combinations thereof, and (iii) an aqueous-solute fluid; and
wherein the one or more polymer or copolymer fragments, or combinations thereof, are present in an amount sufficient to stabilize the particles of one or more hydrophobic agent(s) in said second dispersion.

Embodiment 104. A composition comprising:
a dispersion comprising (i) particles of one or more hydrophobic agent(s) having an average particle size of about 5 m or less, (ii) one or more polymer or copolymer fragments, or combinations thereof, and (iii) an aqueous-solute fluid;
wherein the one or more polymer or copolymer fragments, or combinations thereof, are present in an amount sufficient to stabilize the particles of one or more hydrophobic agent(s) in said dispersion;
said composition produced by a process comprising:
preparing a premix comprising (i) one or more hydrophobic agent(s), (ii) one or more polymers or copolymers, or combinations thereof, (iii) an aqueous-solute fluid, and optionally (iv) one or more additive(s);
subjecting the premix to low energy mixing to form a first dispersion; and
subjecting the first dispersion to ultra-high energy mixing to form a second dispersion;
wherein said composition comprises said second dispersion.

Embodiment 105. A composition comprising:
a dispersion comprising (i) particles of one or more hydrophobic agent(s), (ii) one or more polymer or copolymer fragments, or combinations thereof, and (iii) an aqueous-solute fluid;
wherein the particles of one or more hydrophobic agent(s) are present in an amount from about 0.01% wt. to about 70% wt., the aqueous-solute fluid is present in an amount from about 1.0% wt. to about 98.5% wt., and the one or more polymer or copolymer fragments, or combinations thereof, are present in an amount from about 0.01% wt. to about 10% wt., all based on the total weight of the composition;
wherein the particles of one or more hydrophobic agent(s) (i) have an average particle size from about 100 nm to about 5 m, (ii) have a solubility of less than about 0.1% by weight in water under standard conditions, and (iii) are 85% or more of a size within ±2.0 standard deviations, or within ±1.9 standard deviations, or within ±1.8 standard deviations, or within ±1.7 standard deviations, or within ±1.6 standard deviations, or within ±1.5 standard deviations, of the average particle size; and
wherein the one or more polymer or copolymer fragments, or combinations thereof, are sufficient to stabilize the particles of one or more hydrophobic agent(s) in said dispersion, at a level from about 0.05% w/w to about 70% w/w of said one or more hydrophobic agent(s), in said dispersion;
said composition produced by a process comprising:
preparing a premix comprising (i) one or more hydrophobic agent(s), (ii) one or more polymers or copolymers, or combinations thereof, (iii) an aqueous-solute fluid, and optionally (iv) one or more additive(s);
subjecting the premix to low energy mixing to form a first dispersion; and
subjecting the first dispersion to ultra-high energy mixing to form a second dispersion;
wherein said composition comprises said second dispersion.

Embodiment 106. A method of treating disorders of human or animal skin, hair or mucosal tissue, said method comprising:
applying to the skin, hair or external mucosa of a human or animal a composition comprising:
a dispersion comprising (i) particles of one or more hydrophobic agent(s) having an average particle size of about 5 m or less, (ii) one or more polymer or copolymer fragments, or combinations thereof, and (iii) an aqueous-solute fluid;
wherein the one or more hydrophobic agent(s) comprise one or more therapeutic agent(s); and
wherein the one or more polymer or copolymer fragments, or combinations thereof, are present in an amount sufficient to stabilize the particles of one or more hydrophobic agent(s) in said dispersion.

Embodiment 107. A method of imparting a desirable tactile, olfactory, or visual property to a skin, hair, or mucosal surface of a human or animal, or to a surfaces or substrate, said method comprising:
applying to the skin, hair or external mucosa of a human or animal, or to the surface or substrate, a composition comprising:
a dispersion comprising (i) particles of one or more hydrophobic agent(s) having an average particle size of about 5 m or less, (ii) one or more polymer or fragments, or combinations thereof, and (iii) an aqueous-solute fluid;
wherein the one or more hydrophobic agent(s) comprise one or more aesthetic modifying agent(s); and
wherein the one or more polymer fragments or copolymer fragments, or combinations thereof, are present in an amount sufficient to stabilize the particles of one or more hydrophobic agent(s) in said dispersion.

Embodiment 108. A method of delivering one or more active or therapeutic ingredients to a human or animal, said method comprising:
providing a dispersion comprising (i) particles of one or more hydrophobic agent(s) having an average particle size of about 5 m or less, (ii) one or more polymer or copolymer fragments, or combinations thereof, and (iii) an aqueous-solute fluid;
wherein the one or more polymer or copolymer fragments, or combinations thereof, are present in an amount sufficient to stabilize the particles of one or more hydrophobic agent(s) in said dispersion;
wherein the dispersion acts as a multifunctional delivery vehicle for active or therapeutic ingredients, and
using the multifunctional delivery vehicle to deliver the one or more active or therapeutic ingredients to a human or animal.

Embodiment 109. A method for reducing transient flora on skin and improving the condition of the skin, said method comprising:
applying to the skin of a human or animal a composition comprising:
a dispersion comprising (i) particles of one or more hydrophobic agent(s) having an average particle size of about 5 m or less, (ii) one or more polymer or copolymer fragments, or combinations thereof, and (iii) an aqueous-solute fluid;
wherein the one or more hydrophobic agent(s) comprise one or more therapeutic agent(s) and/or one or more aesthetic modifying agent(s); and
wherein the one or more polymer or copolymer fragments, or combinations thereof, are present in an amount sufficient to stabilize the particles of one or more hydrophobic agent(s) in said dispersion.

Embodiment 110. The method of embodiment 109 wherein the dispersion further comprises one or more antibiotics, bactericides, or antifungal agents, or one or more humectants.

Embodiment 111. A method of using a composition to enhance a physical, chemical, nutritional and/or sensory property of a food, said method comprising:
applying an edible composition into or onto the food, said edible composition comprising:
a dispersion comprising (i) particles of one or more edible hydrophobic agent(s) having an average particle size of about 5 m or less, (ii) one or more edible polymer or copolymer fragments, or combinations thereof, and (iii) an aqueous-solute fluid;
wherein the one or more edible hydrophobic agent(s) comprise one or more edible therapeutic agent(s) and/or one or more edible aesthetic modifying agent(s); and
wherein the one or more edible polymer or copolymer fragments, or combinations thereof, are present in an amount sufficient to stabilize the particles of one or more edible hydrophobic agent(s) in said dispersion;
wherein the particles of the one or more edible hydrophobic agent(s) in the dispersion increase an extent of penetration of the dispersion throughout a water phase of a substrate of the food; and
thereby producing a bloom effect distributing the particles of the one or more edible hydrophobic agent(s) uniformly throughout the water phase of the substrate that enhances a physical, chemical, nutritional and/or sensory property of the food.

Embodiment 112. The method of embodiment 111 wherein the dispersion further comprises one or more edible rheological agents.

Embodiment 113. A method of using a composition to enhance a physical, chemical, nutritional and/or sensory property of a beverage, said method comprising:
applying an edible composition into the beverage, the edible composition comprising:
a dispersion comprising (i) particles of one or more edible hydrophobic agent(s) having an average particle size of about 5 m or less, (ii) one or more edible polymer or copolymer fragments, or combinations thereof, and (iii) an aqueous-solute fluid;
wherein the one or more edible hydrophobic agent(s) comprise one or more edible therapeutic agent(s) and/or one or more edible aesthetic modifying agent(s); and
wherein the one or more edible polymer or copolymer fragments, or combinations thereof, are present in an amount sufficient to stabilize the particles of one or more edible hydrophobic agent(s) in said dispersion;
wherein the particles of the one or more edible hydrophobic agent(s) in the dispersion increase an extent of penetration of the dispersion throughout a water phase of the beverage; and
thereby producing a bloom effect distributing the particles of the one or more edible hydrophobic agent(s) uniformly throughout the water phase of the beverage that enhances a physical, chemical, nutritional and/or sensory property of the beverage.

Embodiment 114. The method of embodiment 113 wherein the dispersion further comprises one or more edible rheological agents.

Embodiment 1115 A method of enhancing food, said method comprising:
contacting the food with a composition comprising:
a dispersion comprising (i) particles of one or more edible hydrophobic agent(s) having an average particle size of about 5 m or less, (ii) one or more edible polymer or copolymer fragments, or combinations thereof, and (iii) an aqueous-solute fluid.

Embodiment 116. The method of embodiment 115 wherein the dispersion further comprises one or more edible rheological agents.

Other illustrative embodiments useful in this disclosure are described, for example, in U.S. Pat. Nos. 9,357,770, 9,980,886, and 10,531,674, and also U.S. patent application Ser. Nos. 13/835,642, 16/701,477, and 17/512,251, all of which are incorporated herein by reference in their entirety.

The following embodiments are intended to demonstrate the versatility of hydrophobic agent dispersions of this disclosure. These examples can be utilized as presented or can be diluted in water or water miscible solvent to a concentration that is optimized for a given application. They can also be combined in various ratios to provide multiple benefits to the consumer.

### EXAMPLES

### General Compositions

A composition formulated as a liquid/toner was prepared and summarized below.

| | | Liquid/Toner |
|---|---|---|
| | | 10 cts |
| | | Dimethicone |
| | | D1 |
| Phase | Ingredient | Percentage |
| A | Water | 58.10 |
| A | Glycerin | 10.00 |
| A | Phenoxyethanol, Ethylhexylglycerin | 0.67 |
| A | Potassium Sorbate | 0.20 |
| A | Sodium Benzoate | 0.20 |
| B | Water & Glycerin & Phenoxyethanol & | 30.83 |
| | Ethylhexylglycerin& Potassium Sorbate & | |
| | Sodium Benzoate & | |
| | Dimethicone & Citric Acid & | |
| | Acrylates/C10-30 Alkyl Acrylate | |
| | Crosspolymer & Sodium Hydroxide (A5) | |
| | Total | 100.00 |

The composition D1 was prepared in accordance with the processes of this disclosure, in particular, Phase A ingredients were combined into a main vessel and propeller mixing started; Phase B was added slowly into the main vessel and mixing continued; then switched to a homogenizer and homogenized at high shear for about 5 min.

A composition formulated as a fluid/serum was prepared and summarized below.

| | | Fluid/Serum |
|---|---|---|
| | | 10 cts |
| | | Dimethicone |
| | | D2 |
| Phase | Ingredient | Percentage |
| A | Water | 65.28 |
| C | Glycerin | 1.00 |
| C | Benzyl Alcohol | 1.00 |
| C | Phenoxyethanol | 1.00 |
| C | 10 cst Dimethicone | 30.00 |
| C | Citric Acid, Water | 0.62 |
| B | Acrylates/C10-30 Alkyl Acrylate | 0.35 |
| | Crosspolymer | |
| B | Hydroxyethyl Acrylate/Sodium | 0.36 |
| | Acryloyldimethyl Taurate Copolymer | |
| D | Aminomethyl Propanol | 0.39 |
| | Total | 100.00 |

The composition D2 was prepared in accordance with the processes of this disclosure, in particular, D.I. water was added into a beaker; Phase B polymers were blended together and sifted into water; then mixed until fully hydrated; Phase C ingredients were added into Phase AB and mixed well and homogenized; Phase ABC made 2 passes through a microfluidizer; and pH adjusted pH 7.00-8.00.

A composition formulated as a lotion was prepared and summarized below.

| | | Lotion |
|---|---|---|
| | | 10 cts |
| | | Dimethicone |
| | | D3 |
| Phase | Ingredient | Percentage |
| A | Water | 56.40 |
| A | Glycerin | 10.00 |
| A | Phenoxyethanol, Ethylhexylglycerin | 1.00 |
| A | Potassium Sorbate | 0.25 |
| A | Sodium Benzoate | 0.25 |
| B | 10 cst Dimethicone | 30.00 |
| C | Citric Acid, Water | 1.00 |
| D | Acrylates/C10-30 Alkyl Acrylate | 0.80 |
| | Crosspolymer | |
| E | Sodium Hydroxide, Water | 0.30 |
| | Total | 100.00 |

The composition D3 was prepared in accordance with the processes of this disclosure, in particular, Phase A and Phase C were combined into a main vessel with propeller mixing; then switched to a homogenizer and Phase D added slowly; then Phase B was added; passed through a microfluidizer twice with cooling; switched main vessel to homogenizer and added Phase F; switched main vessel to propeller mixer and adjusted pH with Phase E; adjusted to pH 6.00-6.50.

A composition formulated as a soft cream was prepared and summarized below.

| | | Soft Cream |
|---|---|---|
| | | 10 cts |
| | | Dimethicone |
| | | D4 |
| Phase | Ingredient | Percentage |
| A | Water | 56.40 |
| A | Glycerin | 10.00 |
| A | Phenoxyethanol, Ethylhexylglycerin | 1.00 |
| A | Potassium Sorbate | 0.25 |
| A | Sodium Benzoate | 0.25 |
| B | 10 cst Dimethicone | 30.00 |
| C | Citric Acid, Water | 1.00 |
| D | Acrylates/C10-30 Alkyl Acrylate | 0.80 |
| | Crosspolymer | |
| E | Sodium Hydroxide, Water | 0.30 |
| | Total | 100.00 |

The composition D4 was prepared in accordance with the processes of this disclosure, in particular, Phase A and Phase C were combined into a main vessel with propeller mixing; switched main vessel to homogenizer and added Phase D slowly; then added Phase B; passed through a microfluidizer twice with cooling; switched main vessel to homogenizer and added Phase F; switched main vessel to propeller mixer and adjusted pH with Phase E; adjusted to pH 6.00-6.50.

A composition formulated as a rich cream was prepared and summarized below.

| | | Rich Cream |
|---|---|---|
| | | 10 cts |
| | | Dimethicone |
| | | D5 |
| Phase | Ingredient | Percentage |
| A | Water | 56.40 |
| A | Glycerin | 10.00 |
| A | Phenoxyethanol, Ethylhexylglycerin | 1.00 |
| A | Potassium Sorbate | 0.25 |
| A | Sodium Benzoate | 0.25 |
| B | 10 cst Dimethicone | 30.00 |
| C | Citric Acid, Water | 1.00 |
| D | Acrylates/C10-30 Alkyl Acrylate | 0.80 |
| | Crosspolymer | |
| E | Sodium Hydroxide, Water | 0.30 |
| | Total | 100.00 |

The composition D5 was prepared in accordance with the processes of this disclosure, in particular, Phase A and Phase C were combined into a main vessel with propeller mixing; switched main vessel to homogenizer and added Phase D slowly; added Phase B; passed through a microfluidizer twice with cooling; switched main vessel to homogenizer and added Phase F; switched main vessel to propeller mixer and adjusted pH with Phase E; adjusted to pH 6.00-6.50.

A composition formulated as a rich cream was prepared and summarized below.

| | | Rich Cream |
|---|---|---|
| | | 10 cts |
| | | Dimethicone |
| | | D6 |
| Phase | Ingredient | Percentage |
| A | Water | 53.90 |
| A | Glycerin | 10.00 |
| A | Phenoxyethanol, Ethylhexylglycerin | 1.00 |
| A | Potassium Sorbate | 0.25 |
| A | Sodium Benzoate | 0.25 |
| B | 10 cst Dimethicone | 30.00 |
| B | Citric Acid, Water | 1.30 |
| C | Acrylates/C10-30 Alkyl Acrylate | 0.60 |
| | Crosspolymer (Fluidized) | |
| D | Acrylates/C10-30 Alkyl Acrylate | 0.20 |
| | Crosspolymer (Non-fluidized) | |
| E | Sodium Hydroxide, Water | 2.50 |
| | Total | 100.00 |

The composition D6 was prepared in accordance with the processes of this disclosure, in particular, Phase A was added to a main kettle and propeller mixing started until uniform; Phase B charged into main kettle and continued mixing; Phase C charged into main kettle and then slowly sifted Phase D into main kettle under homogenization; mixed until uniform and then passed through homogenizer for 2 passes.

A composition formulated as a spray was prepared and summarized below.

| | | Spray |
|---|---|---|
| | | 10 cts |
| | | Dimethicone |
| | | D7 |
| Phase | Ingredient | Percentage |
| A | Water | 58.10 |
| A | Glycerin | 10.00 |
| A | Phenoxyethanol, Ethylhexylglycerin | 0.67 |
| A | Potassium Sorbate | 0.20 |
| A | Sodium Benzoate | 0.20 |
| B | Water & Glycerin & Phenoxyethanol & | 30.83 |
| | Ethylhexylglycerin& Potassium Sorbate & | |
| | Sodium Benzoate & | |
| | Dimethicone & Citric Acid & | |
| | Acrylates/C10-30 Alkyl Acrylate | |
| | Crosspolymer & Sodium Hydroxide (A4) | |
| | Total | 100.00 |

The composition D7 was prepared in accordance with the processes of this disclosure, in particular, Phase A was combined into a main vessel and propeller mixing started; Phase B was added slowly into main vessel and continued mixing; switched to homogenizer and homogenized at high shear for about 5 min.

A composition formulated as a spray was prepared and summarized below.

| | | Spray |
|---|---|---|
| | | 10 cts |
| | | Dimethicone |
| | | D8 |
| Phase | Ingredient | Percentage |
| A | Water | 73.36 |
| C | Glycerin | 2.50 |
| C | Benzyl Alcohol | 0.50 |
| C | Phenoxyethanol | 0.50 |
| C | Phenyl Trimethicone | 8.25 |
| C | Dimethicone- High Viscosity Silicone | 8.25 |
| | (Gransil 530) | |
| C | Dimethicone- Low Viscosity Silicone | 5.13 |
| | (Botanisil DM-80) | |
| F | Citric Acid, Water | 0.57 |
| B | Acrylates/C10-30 Alkyl Acrylate | 0.25 |
| | Crosspolymer | |
| B | Hydroxyethyl Acrylate/Sodium | 0.26 |
| | Acryloyldimethyl Taurate Copolymer | |
| F | Aminomethyl Propanol | 0.44 |
| | Total | 100.00 |

The composition D8 was prepared in accordance with the processes of this disclosure, in particular, D.I. water was measured into a main beaker; polymers wee blended together, added into water, and mixed until fully hydrated; ingredients of Phase C were added into Phase AB, and mixed well; Phase ABCD made 2 passes through a microfluidizer; adjusted pH; added D.I. water, and homogenized.

A composition formulated as a liquid/toner was prepared and summarized below.

| | | Liquid/Toner |
|---|---|---|
| | | Caprylic/capric |
| | | triglyceride |
| | | D9 |
| Phase | Ingredient | Percentage |
| A | Water | 58.10 |
| A | Glycerin | 10.00 |
| A | Phenoxyethanol, Ethylhexylglycerin | 0.67 |
| A | Potassium Sorbate | 0.20 |
| A | Sodium Benzoate | 0.20 |
| B | Water & Glycerin & Phenoxyethanol & | 30.83 |
| | Ethylhexylglycerin & Potassium Sorbate & | |
| | Sodium Benzoate & Caprylic/Capric | |
| | Triglyceride & Citric Acid & Acrylates/C10- | |
| | 30 Alkyl Acrylate Crosspolymer & Sodium | |
| | Hydroxide (A3) | |
| | Total | 100.00 |

The composition D9 was prepared in accordance with the processes of this disclosure, in particular, started mixing Phase A with propeller mixer; added Phase B to a main vessel; added Phase C to main vessel; took the batch and filled a spray bottle; sprayed.

A composition formulated as a fluid/serum was prepared and summarized below.

| | | Fluid/Serum |
|---|---|---|
| | | Caprylic/capric |
| | | triglyceride |
| | | D10 |
| Phase | Ingredient | Percentage |
| A | Water | 64.90 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | Caprylic/capric triglyceride | 20.00 |
| B | Citric Acid, Water | 2.50 |
| D | Acrylates/C10-30 Alkyl | 0.80 |
| | Acrylate Crosspolymer | |
| F | Sodium Hydroxide, Water | 0.30 |
| | Total | 100.00 |

The composition D10 was prepared in accordance with the processes of this disclosure, in particular, Phase A and Phase B were combined into a main vessel with propeller mixing; switched from main vessel to homogenizer and added Phase D slowly; added Phase C; passed through a microfluidizer twice with cooling; switched main vessel to homogenizer and added Phase E; switched main vessel to propeller mixer and adjusted pH with Phase F; adjusted to pH 6.00.

A composition formulated as a lotion was prepared and summarized below.

| | | Lotion |
|---|---|---|
| | | Caprylic/capric |
| | | triglyceride |
| | | D11 |
| Phase | Ingredient | Percentage |
| A | Water | 56.40 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | Caprylic/capric triglyceride | 30.00 |
| B | Citric Acid, Water | 1.00 |
| D | Acrylates/C10-30 Alkyl | 0.80 |
| | Acrylate Crosspolymer | |
| E | Sodium Hydroxide, Water | 0.30 |
| | Total | 100.00 |

The composition D11 was prepared in accordance with the processes of this disclosure, in particular, Phase A and Phase were combined into a main vessel with propeller mixing; switched main vessel to homogenizer and added Phase D slowly; added Phase C; passed through a microfluidizer twice with cooling; switched main vessel to homogenizer and added Phase F; switched main vessel to propeller mixer and adjusted pH with Phase E; adjusted to pH 6.00-6.50.

A composition formulated as a soft cream was prepared and summarized below.

| | | Soft Cream |
|---|---|---|
| | | Caprylic/capric |
| | | triglyceride |
| | | D12 |
| Phase | Ingredient | Percentage |
| A | Water | 56.40 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | Caprylic/capric triglyceride | 30.00 |
| B | Citric Acid, Water | 1.00 |
| D | Acrylates/C10-30 Alkyl | 0.80 |
| | Acrylate Crosspolymer | |
| E | Sodium Hydroxide, Water | 0.30 |
| | Total | 100.00 |

The composition D12 was prepared in accordance with the processes of this disclosure, in particular, Phase A and Phase B were combined into a main vessel with propeller mixing; switched main vessel to a homogenizer and added Phase D slowly; added Phase C; passed through a microfluidizer twice with cooling; switched main vessel to homogenizer and added Phase F; switched main vessel to propeller mixer and adjusted pH with Phase E; adjusted to pH 6.00-6.50.

A composition formulated as a rich cream was prepared and summarized below.

| | | Rich Cream |
|---|---|---|
| | | Caprylic/capric |
| | | triglyceride |
| | | D13 |
| Phase | Ingredient | Percentage |
| A | Water | 56.40 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | Caprylic/capric triglyceride | 30.00 |
| B | Citric Acid, Water | 1.00 |
| D | Acrylates/C10-30 Alkyl | 0.80 |
| | Acrylate Crosspolymer | |
| E | Sodium Hydroxide, Water | 0.30 |
| | Total | 100.00 |

The composition D13 was prepared in accordance with the processes of this disclosure, in particular, Phase A and Phase B were combined into a main vessel with propeller mixing; switched main vessel to a homogenizer and added Phase D slowly; added Phase C; passed through a microfluidizer twice with cooling; switched main vessel to homogenizer and added Phase F; switched main vessel to propeller mixer and adjusted pH with Phase E; adjusted to pH 6.00-6.50.

A composition formulated as a spray was prepared and summarized below.

| | | Spray |
|---|---|---|
| | | Caprylic/capric |
| | | triglyceride |
| | | D14 |
| Phase | Ingredient | Percentage |
| A | Water | 58.10 |
| A | Glycerin | 10.00 |
| A | Phenoxyethanol, | 0.67 |
| | Ethylhexylglycerin | |
| A | Potassium Sorbate | 0.20 |
| A | Sodium Benzoate | 0.20 |
| B | Water & Glycerin & Phenoxyethanol & | 30.83 |
| | Ethylhexylglycerin & Potassium Sorbate & | |
| | Sodium Benzoate & Caprylic/Capric | |
| | Triglyceride & Citric Acid & Acrylates/C10- | |
| | 30 Alkyl Acrylate Crosspolymer & Sodium | |
| | Hydroxide (A2) | |
| | Total | 100.00 |

The composition D14 was prepared in accordance with the processes of this disclosure, in particular, started mixing Phase A with a propeller mixer; added Phase B to a main vessel; added Phase C to main vessel; filled a spray bottle with the batch and sprayed.

A composition formulated as a liquid/toner was prepared and summarized below.

| | | Liquid/Toner |
|---|---|---|
| | | Olive Oil |
| | | D15 |
| Phase | Ingredient | Percentage |
| A | Water | 57.20 |
| C | Glycerin | 10.00 |
| C | Benzyl Alcohol | 1.00 |
| D | Jojoba Oil | 30.00 |
| D | Tocopherol Acetate | 1.00 |
| E | Citric Acid, Water | Q.S. |
| B | Acrylates/C10-30 Alkyl | 0.40 |
| | Acrylate Crosspolymer | |
| B | Hydroxyethyl Acrylate/Sodium | 0.40 |
| | Acryloyldimethyl Taurate Copolymer | |
| E | Aminomethyl Propanol | Q.S. |
| | Total | 100.00 |

The composition D15 was prepared in accordance with the processes of this disclosure, in particular, measured D.I. water into beaker; blended polymers together and added into water; mixed until fully hydrated; added ingredients of Phase C into Phase AB; mixed well; premix Phase D; added into Phase ABC by portions; homogenized well with three passes through a microfluidizer; adjusted pH in a range 6.75-7.25.

A composition formulated as a fluid/serum was prepared and summarized below.

| | | Fluid/Serum |
|---|---|---|
| | | Olive Oil |
| | | D16 |
| Phase | Ingredient | Percentage |
| A | Water | 58.85 |
| C | Glycerin | 7.50 |
| C | Benzyl Alcohol | 1.00 |
| C | Phenoxyethanol | 1.00 |
| C | 10 cst Dimethicone | 15.00 |
| C | Soybean Oil | 12.50 |
| C | Stearic Acid | 0.15 |
| D | Echium Plantagineum Seed Oil, Tocopherol, | 2.50 |
| | Lecithin, Ascorbyl Palmitate, Glyceryl | |
| | Stearate, Glyceryl Oleate, Citric Acid | |
| | Tocopherol | 0.50 |
| E | Citric Acid, Water | Q.S |
| B | Acrylates/C10-30 Alkyl | 0.50 |
| | Acrylate Crosspolymer | |
| B | Hydroxyethyl Acrylate/Sodium | 0.50 |
| | Acryloyldimethyl Taurate Copolymer | |
| E | Aminomethyl Propanol | Q.S |
| | Total | 100.00 |

The composition D16 was prepared in accordance with the processes of this disclosure, in particular, D.I. water was measured in to main beaker; blended polymers together and added into water; mixed until fully hydrated; ingredients of Phase C were added into Phase AB; mixed well; combined Phase D and heated up to 72° C. to melt stearic acid; added Phase D into Phase ABC, and homogenized 3 passes through a microfluidizer; adjusted pH up to 6.85-7.50.

A composition formulated as a lotion was prepared and summarized below.

| | | Lotion |
|---|---|---|
| | | Olive Oil |
| | | D17 |
| Phase | Ingredient | Percentage |
| A | Water | 50.20 |
| A | Glycerin | 5.00 |
| A | Propanediol | 10.00 |
| A | Phenoxyethanol, | 1.00 |
| | Ethylhexylglycerin | |
| A | Potassium Sorbate | 0.25 |
| A | Sodium Benzoate | 0.25 |
| B | Olive Oil | 30.00 |
| C | Citric Acid, Water | 1.00 |
| D | Acrylates/C10-30 Alkyl | 0.80 |
| | Acrylate Crosspolymer | |
| E | Sodium Hydroxide, Water | 1.50 |
| | Total | 100.00 |

The composition D17 was prepared in accordance with the processes of this disclosure, in particular, Phase A and Phase C were combined into a main vessel with propeller mixing; switched main vessel to a homogenizer and added Phase D slowly; added Phase B; passed through a microfluidizer twice with cooling; switched main vessel to homogenizer and added Phase F; switched main vessel to propeller mixer and adjusted pH with Phase E; adjusted to pH 6.00-6.50.

A composition formulated as a soft cream was prepared and summarized below.

| | | Soft Cream |
|---|---|---|
| | | Olive Oil |
| | | D18 |
| Phase | Ingredient | Percentage |
| A | Water | 50.20 |
| A | Glycerin | 5.00 |
| A | Propanediol | 10.00 |
| A | Phenoxyethanol, | 1.00 |
| | Ethylhexylglycerin | |
| A | Potassium Sorbate | 0.25 |
| A | Sodium Benzoate | 0.25 |
| B | Olive Oil | 30.00 |
| C | Citric Acid, Water | 1.00 |
| D | Acrylates/C10-30 Alkyl | 0.80 |
| | Acrylate Crosspolymer | |
| E | Sodium Hydroxide, Water | 1.50 |
| | Total | 100.00 |

The composition D18 was prepared in accordance with the processes of this disclosure, in particular, Phase A and Phase C were combined into a main vessel with propeller mixing; switched main vessel to homogenizer and added Phase D slowly; added Phase B; passed through a microfluidizer twice with cooling; switched main vessel to homogenizer and added Phase F; switched main vessel to propeller mixer and adjusted pH with Phase E; adjusted to pH 6.00-6.50.

A composition formulated as a rich cream was prepared and summarized below.

| | | Rich Cream |
|---|---|---|
| | | Olive Oil |
| | | D19 |
| Phase | Ingredient | Percentage |
| A | Water | 50.20 |
| A | Glycerin | 5.00 |
| A | Propanediol | 10.00 |
| A | Phenoxyethanol, | 1.00 |
| | Ethylhexylglycerin | |
| A | Potassium Sorbate | 0.25 |
| A | Sodium Benzoate | 0.25 |
| B | Olive Oil | 30.00 |
| C | Citric Acid, Water | 1.00 |
| D | Acrylates/C10-30 Alkyl | 0.80 |
| | Acrylate Crosspolymer | |
| E | Sodium Hydroxide, Water | 1.50 |
| | Total | 100.00 |

The composition D19 was prepared in accordance with the processes of this disclosure, in particular, Phase A and Phase C were combined into a main vessel with propeller mixing; switched main vessel to a homogenizer and added Phase D slowly; added Phase B; passed through a microfluidizer twice with cooling; switched main vessel to homogenizer and added Phase F; switched main vessel to propeller mixer and adjusted pH with Phase E; adjusted to pH 6.00-6.50.

A composition formulated as a spray was prepared and summarized below.

| | | Spray |
|---|---|---|
| | | Olive Oil |
| | | D20 |
| Phase | Ingredient | Percentage |
| A | Water | 57.20 |
| C | Glycerin | 10.00 |
| C | Benzyl Alcohol | 1.00 |
| D | Jojoba Oil | 30.00 |
| D | Tocopherol Acetate | 1.00 |
| E | Citric Acid, Water | Q.S |
| B | Acrylates/C10-30 Alkyl | 0.40 |
| | Acrylate Crosspolymer | |
| B | Hydroxyethyl Acrylate/Sodium | 0.40 |
| | Acryloyldimethyl | |
| | Taurate Copolymer | |
| E | Aminomethyl Propanol | Q.S |
| | Total | 100.00 |

The composition D20 was prepared in accordance with the processes of this disclosure, in particular, D.I. water was measured into a beaker; blended polymers together and added into water; mixed until fully hydrated; added ingredients of Phase C into Phase AB; mixed well; premix Phase D; added in to Phase ABC by portions; homogenized well; 3 passes made through microfluidizer; adjusted pH in a range 6.75-7.25.

A composition formulated as a liquid/toner was prepared and summarized below.

| | | Liquid/Toner |
|---|---|---|
| | | C12-15 Alkyl |
| | | Benzoate |
| | | D21 |
| Phase | Ingredient | Percentage |
| A | Water | 54.90 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | C12-15 Alkyl Benzoate | 30.00 |
| B | Citric Acid, Water | 2.50 |
| D | Acrylates/C10-30 Alkyl | 0.80 |
| | Acrylate Crosspolymer | |
| F | Sodium Hydroxide, Water | 0.30 |
| | Total | 100.00 |

The composition D21 was prepared in accordance with the processes of this disclosure, in particular, Phase A and Phase B were combined into a main vessel with propeller mixing; switched main vessel to a homogenizer and added Phase D slowly; added Phase C; passed through the a microfluidizer twice with cooling; switched main vessel to homogenizer and added Phase E; switched main vessel to propeller mixer and adjusted pH with Phase F; adjusted to pH 6.00.

A composition formulated as a liquid/toner was prepared and summarized below.

| | | Liquid/Toner |
|---|---|---|
| | | C12-15 Alkyl |
| | | Benzoate |
| | | D22 |
| Phase | Ingredient | Percentage |
| A | Water | 60.48 |
| C | Glycerin | 7.50 |
| C | Benzyl Alcohol | 1.00 |
| D | C12-15 Alkyl Benzoate | 30.00 |
| E | Citric Acid, Water | Q.S |
| B | Acrylates/C10-30 Alkyl | 0.75 |
| | Acrylate Crosspolymer | |
| E | Aminomethyl Propanol | 0.28 |
| | Total | 100.00 |

The composition D22 was prepared in accordance with the processes of this disclosure, in particular, D.I. water was measured and added into a beaker; blended polymers together and added into water; mixed until fully hydrated and homogenized; added ingredients of Phase C into Phase AB; mixed well; added Phase D in to Phase ABC; mixed for 20 min. and homogenized well; two passes made through microfluidizer; adjusted pH in a range 5.20-5.35.

A composition formulated as a fluid/serum was prepared and summarized below.

| | | Fluid/Serum |
|---|---|---|
| | | C12-15 Alkyl |
| | | Benzoate |
| | | D23 |
| Phase | Ingredient | Percentage |
| A | Water | 54.90 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | C12-15 Alkyl Benzoate | 30.00 |
| B | Citric Acid, Water | 2.50 |
| D | Acrylates/C10-30 Alkyl | 0.80 |
| | Acrylate Crosspolymer | |
| F | Sodium Hydroxide, Water | 0.30 |
| | Total | 100.00 |

The composition D23 was prepared in accordance with the processes of this disclosure, in particular, Phase A and Phase B were combined into a main vessel with propeller mixing; switched main vessel to a homogenizer and added Phase D slowly; added Phase C; passed through a microfluidizer twice with cooling; switched main vessel to homogenizer and added Phase E; switched main vessel to propeller mixer and adjusted pH with Phase F; adjusted to pH 6.00.

A composition formulated as a lotion was prepared and summarized below.

| | | Lotion |
|---|---|---|
| | | C12-15 Alkyl |
| | | Benzoate |
| | | D24 |
| Phase | Ingredient | Percentage |
| A | Water | 60.64 |
| A | Propanediol | 5.00 |
| B | Pentylene Glycol | 4.50 |
| A | Benzyl Alcohol | 0.75 |
| A | Silica | 1.83 |
| A | Trehalose | 2.00 |
| A | Sucrose | 2.00 |
| A | Benzoyl Peroxide | 13.12 |
| B | Tetrasodium ethylenediamine | 0.05 |
| | tetraacetate, Sodium | |
| | Hydroxide Water | |
| A | Butylene Glycol, Water, | 0.10 |
| | Centella Astatica Extract, | |
| | Madecassoside, Asiatlcoside | |
| B | Xanthan Gum | 0.54 |
| C | Monosodium Citrate | 0.17 |
| D | Water (and) Acrylates/C10-30 Alkyl | 9.30 |
| | Acrylate Crosspolymer (and) Glycerin (and) | |
| | Benzyl Alcohol (and) C12-15 Alkyl | |
| | Benzoate (and) Aminomethyl Propanol (and) | |
| | Citric Acid (A1) | |
| | Total | 100.00 |

The composition D24 was prepared in accordance with the processes of this disclosure, in particular, ingredients of Phase A were combined together; mixed well for 10 min; premix Phase B added to water phase; mixed 30 min; heated Phase C up to 50° C.-65° C. while mixing until clear; added Phase D; mixed well for 20 min. and homogenized.

A composition formulated as a soft cream was prepared and summarized below.

| | | Soft Cream |
|---|---|---|
| | | C12-15 Alkyl |
| | | Benzoate |
| | | D25 |
| Phase | Ingredient | Percentage |
| A | Water | 54.90 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | C12-15 Alkyl Benzoate | 30.00 |
| B | Citric Acid, Water | 2.50 |
| D | Acrylates/C10-30 | 0.80 |
| | Alkyl Acrylate | |
| | Crosspolymer | |
| F | Sodium Hydroxide, Water | 0.30 |
| | Total | 100.00 |

The composition D25 was prepared in accordance with the processes of this disclosure, in particular, Phase A and Phase B were combined into a main vessel with propeller mixing; switched main vessel to a homogenizer and added Phase D slowly; added Phase C; passed through a microfluidizer twice with cooling; switched main vessel to homogenizer and added Phase E; switched main vessel to propeller mixer and adjusted pH with Phase F; adjusted to pH 6.00.

A composition formulated as a soft cream was prepared and summarized below.

| | | Soft Cream |
|---|---|---|
| | | C12-15 Alkyl |
| | | Benzoate |
| | | D26 |
| Phase | Ingredient | Percentage |
| A | Water | 49.83 |
| B | Glycerin | 4.50 |
| B | Pentylene Glycol | 4.50 |
| D | Benzyl Alcohol | 0.75 |
| A | Trehalose | 2.00 |
| A | Sucrose | 2.00 |
| C | Benzoyl Peroxide | 26.22 |
| A | Tetrasodium ethylenediamine tetraacetate, | 0.05 |
| | Sodium Hydroxide Water | |
| A | Butylene Glycol, Water, Centella Astatica | 0.10 |
| | Extract, Madecassoside, Asiatlcoside | |
| D | Carbomer | 0.75 |
| D | Water (and) Acrylates/C10-30 Alkyl | 9.30 |
| | Acrylate Crosspolymer (and) Glycerin (and) | |
| | Benzyl Alcohol (and) C12-15 Alkyl | |
| | Benzoate (and) Aminomethyl Propanol (and) | |
| | Citric Acid (A1) | |
| | Total | 100.00 |

The composition D26 was prepared in accordance with the processes of this disclosure, in particular, the ingredients of Phase A were combined together; mixed well for 10 min; premix Phase B added to water phase; mixed 30 min; added Phase C; mixed for 10 min; added Phase D; mixed well for 20 min. and homogenized.

A composition formulated as a rich cream was prepared and summarized below.

| | | Rich Cream |
|---|---|---|
| | | C12-15 Alkyl |
| | | Benzoate |
| | | D27 |
| Phase | Ingredient | Percentage |
| A | Water | 61.98 |
| B | Glycerin | 5.00 |
| B | Pentylene Glycol | 4.50 |
| D | Benzyl Alcohol | 0.75 |
| A | Trehalose | 2.00 |
| A | Sucrose | 2.00 |
| C | Benzoyl Peroxide | 13.12 |
| A | Tetrasodium ethylenediamine tetraacetate, | 0.05 |
| | Sodium Hydroxide Water | |
| A | Butylene Glycol, Water, Centella Astatica | 0.10 |
| | Extract, Madecassoside, Asiatlcoside | |
| B | Carbomer | 1.20 |
| D | Water (and) Acrylates/C10-30 Alkyl | 9.30 |
| | Acrylate Crosspolymer (and) Glycerin (and) | |
| | Benzyl Alcohol (and) C12-15 Alkyl | |
| | Benzoate (and) Aminomethyl Propanol (and) | |
| | Citric Acid (A1) | |
| | Total | 100.00 |

The composition D27 was prepared in accordance with the processes of this disclosure, in particular, the ingredients of Phase A were combined together; mixed well for 10 min; premix Phase B added to water phase; mixed 30 min; added Phase C; mixed for 10 min; added Phase D; mixed well for 20 min. and homogenized.

A composition formulated as a spray was prepared and summarized below.

| | | Spray |
|---|---|---|
| | | C12-15 Alkyl |
| | | Benzoate |
| | | D28 |
| Phase | Ingredient | Percentage |
| A | Water | 58.10 |
| A | Glycerin | 10.00 |
| A | Phenoxyethanol, Ethylhexylglycerin | 0.67 |
| A | Potassium Sorbate | 0.20 |
| A | Sodium Benzoate | 0.20 |
| B | Water & Glycerin & Phenoxyethanol & | 30.83 |
| | Ethylhexylglycerin& Potassium Sorbate & | |
| | Sodium Benzoate & | |
| | Dimethicone & Citric Acid & | |
| | Acrylates/C10-30 Alkyl | |
| | Acrylate Crosspolymer & | |
| | Sodium Hydroxide (A4) | |
| | Total | 100.00 |

The composition D28 was prepared in accordance with the processes of this disclosure, in particular, started mixing Phase A with propeller mixer; added Phase B to a main vessel; took the batch, filled a spray bottle, and sprayed.

A composition formulated as a liquid/toner was prepared and summarized below.

| | | Liquid/Toner |
|---|---|---|
| | | Chemical |
| | | UV filters |
| | | D29 |
| Phase | Ingredient | Percentage |
| A | Water | 55.88 |
| A | Glycerin | 7.50 |
| A | Sodium Benzoate | 0.50 |
| A | Benzyl Alcohol | 1.10 |
| B | Octocrylene | 32.00 |
| B | Diethylhexyl Syringylidenemalonate (and) | 1.67 |
| | Caprylic/Capric Triglyceride | |
| D | Citric Acid, Water | 0.10 |
| A | Acrylates/C10-30 Alkyl | 0.75 |
| | Acrylate Crosspolymer | |
| C | Hydroxyethyl Acrylate/Sodium | 0.50 |
| | Acryloyldimethyl Taurate Copolymer | |
| | Total | 100.00 |

The composition D29 was prepared in accordance with the processes of this disclosure, in particular, Phase A ingredients were combined in a main vessel and homogenizing was started with a fine emulsification screen; added Phase B to Phase A and continued homogenizing until uniform; passed through a microfluidizer twice with cooling; slowly sprinkled Phase C into the main vessel under homogenization and continued homogenizing for about 5-10 minutes at moderate speed (6.0-7.0); mixed with propeller for an additional 30 minutes or until completely dispersed and hydrated; adjusted the pH to about 4.50-5.0 using Phase D.

A composition formulated as a fluid/serum was prepared and summarized below.

| | | Fluid/Serum |
|---|---|---|
| | | Chemical |
| | | UV filters |
| | | D30 |
| Phase | Ingredient | Percentage |
| A | Water | 41.66 |
| A | Glycerin | 3.00 |
| A | Sodium Benzoate | 0.38 |
| A | Phenoxyethanol | 1.10 |
| B | Octocrylene | 18.18 |
| B | Ethylhexyl Salicylate | 9.09 |
| B | Diethylhexyl Syringylidenemalonate (and) | 1.50 |
| | Caprylic/Capric Triglyceride | |
| B | Homosalate | 18.18 |
| B | Butyl Methoxydibenzoylmethane | 5.46 |
| D | Citric Acid, Water | 0.20 |
| A | Acrylates/C10-30 Alkyl | 0.75 |
| | Acrylate Crosspolymer | |
| C | Hydroxyethyl Acrylate/Sodium | 0.50 |
| | Acryloyldimethyl Taurate Copolymer | |
| | Total | 100.00 |

The composition D30 was prepared in accordance with the processes of this disclosure, in particular, Phase A ingredients were combined in a main vessel and homogenizing was started with the fine emulsification screen; in a separate vessel, heated (approx. 60° C.-75° C.) and mixed Phase B until completely dissolved and clear; cooled below 45° C.; added Phase B (45° C. or below) to Phase A and continued homogenizing until uniform; passed through a microfluidizer three times with cooling; slowly sprinkled Phase C into the main vessel under homogenization and continued homogenizing for about 5-10 minutes at moderate speed (6.0-7.0); mixed with propeller for an additional 30 minutes or until completely dispersed and hydrated.

A composition formulated as a lotion was prepared and summarized below.

| | | Lotion |
|---|---|---|
| | | Chemical |
| | | UV filters |
| | | D31 |
| Phase | Ingredient | Percentage |
| A | Water | 52.96 |
| B | Glycerin | 10.00 |
| B | Potassium Sorbate | 0.20 |
| B | Phenoxyethanol | 1.25 |
| B | Diethylhexyl Syringylidenemalonate (and) | 3.50 |
| | Caprylic/Capric Triglyceride | |
| B | Polyester-8 | 10.00 |
| B | Ethylhexyl Methoxycrylene | 20.00 |
| C | Citric Acid, Water | 0.42 |
| A | Acrylates/C10-30 Alkyl | 0.75 |
| | Acrylate Crosspolymer | |
| C | Aminomethyl Propanol | 0.93 |
| | Total | 100.00 |

The composition D31 was prepared in accordance with the processes of this disclosure, in particular, sifted Pemulen TR-2 in to water, mixed for 45-60 min until hydrated; added Phase B ingredients into gel Phase A and mixed for 60 min; added citric acid and mixed for another 45 min; Phases ABC mixed well with no homogenizing until smooth lotion is obtained; 2 passes through a microfluidizer; adjusted pH.

A composition formulated as a soft cream was prepared and summarized below.

| | | Soft Cream |
|---|---|---|
| | | Chemical |
| | | UV filters |
| | | D32 |
| Phase | Ingredient | Percentage |
| A | Water | 48.746 |
| A | Glycerin | 5.000 |
| B | Potassium Sorbate | 0.250 |
| B | Sodium Benzoate | 0.250 |
| C | Octocrylene | 13.340 |
| C | Ethylhexyl Salicylate | 6.667 |
| C | Diethylhexyl Syringylidenemalonate (and) | 1.340 |
| | Caprylic/Capric Triglyceride | |
| C | Homosalate | 9.340 |
| C | Butyl Methoxydibenzoylmethane | 4.000 |
| C | Butyloctyl Salicylate | 6.667 |
| E | Acrylates/C10-30 Alkyl | 0.400 |
| | Acrylate Crosspolymer | |
| F | Caprylhydroxamic Acid (and) 1,2- | 2.000 |
| | Hexanediol (and) Propanediol | |
| D | Caprylhydroxamic Acid (and) Caprylyl | 1.000 |
| | Glycol (and) Propanediol | |
| G | Tromethamine (and) Water | 1.000 |
| | Total | 100.000 |

The composition D32 was prepared in accordance with the processes of this disclosure, in particular, added Phase A to a main kettle and began propeller mixing; added phase B and continued propeller mixing; in a side kettle, added Phase C and began heating to about 60-70° C. and propeller mixed until side kettle was fully homogenous; while side kettle was mixing, heated main kettle slightly to about 30-40° C.; once side kettle was homogenous, added it to the main kettle and began cooling the main kettle; sifted Phase D into kettle and allowed to propeller mix first before homogenizing batch until product looked homogenous; processed batch through a microfluidizer for 2 passes and returned batch to propeller mixing; sifted Phase E into kettle under propeller mixing then homogenized until uniform, at about 65-90° C.; cooled batch down once again when this temperature was reached and phase E added into the batch; once batch was under about 30° C., added phase G.

A composition formulated as a rich cream was prepared and summarized below.

| | | Rich Cream |
|---|---|---|
| | | Chemical |
| | | UV filters |
| | | D33 |
| Phase | Ingredient | Percentage |
| A | Water | 69.00 |
| A | Glycerin | 5.00 |
| A | Benzyl Alcohol | 0.95 |
| B | Octocrylene | 6.00 |
| B | Ethylhexyl Salicylate | 4.00 |
| B | Diethylhexyl Syringylidenemalonate (and) | 1.00 |
| | Caprylic/Capric Triglyceride | |
| B | Homosalate | 5.00 |
| B | Butyl Methoxydibenzoylmethane | 2.00 |
| B | Butyloctyl Salicylate | 4.00 |
| A | Citric Acid, Water | 1.00 |
| A | Acrylates/C10-30 Alkyl | 0.40 |
| | Acrylate Crosspolymer | |
| D | Hydroxyethyl Acrylate/Sodium | 0.40 |
| | Acryloyldimethyl Taurate Copolymer | |
| D | Xanthan Gum | 0.30 |
| C | Aminomethyl Propanol | 0.95 |
| | Total | 100.00 |

The composition D33 was prepared in accordance with the processes of this disclosure, in particular, added D.I. water Phase A to main beaker and began propeller mixing; sifted acrylates/C₁₀-30 alkyl acrylate crosspolymer. mix for 30 min to hydrate polymer; added remaining ingredients of Phase A; combined Phase B together in side beaker; heated up to 70° C.-75° C., and mixed well until clear; cooled to about 30-40° C.; added Phase B into Phase A; homogenized for 2 min. and run through a microfluidizer twice; adjusted pH after; sifted xanthan gum and hydroxyethyl acrylate/sodium acryloyldimethyl taurate copolymer of Phase D into side beaker; hydrated well while mixing; added Phase ABC into Phase D and mixed well; adjusted pH and homogenized.

A composition formulated as a spray was prepared and summarized below.

| | | Spray |
|---|---|---|
| | | Chemical |
| | | UV filters |
| | | D34 |
| Phase | Ingredient | Percentage |
| A | Water | 42.81 |
| B | Glycerin | 15.00 |
| B | Potassium Sorbate | 0.20 |
| A | Benzyl Alcohol | 1.00 |
| C | C12-15 Alkyl Benzoate | 5.00 |
| B | Citric Acid, Water | 3.34 |
| B | Acrylates/C10-30 Alkyl | 0.75 |
| | Acrylate Crosspolymer | |
| C | Cyclopentasioxane (and) titanium dioxide | 30.00 |
| | (and) polyglyceryl-3- | |
| | polydimethylsiloxylethyl dimethicone (and) | |
| | aluminum hydroxide (and) stearic acid | |
| D | Aminomethyl Propanol | 1.70 |
| | Total | 100.00 |

The composition D34 was prepared in accordance with the processes of this disclosure, in particular, charged Deionized Water to an appropriate size beaker, stock pot pail or drum and began propeller mixing; to a main vessel, charged: benzyl alcohol, potassium sorbate, sodium benzoate and citric acid 30% aqueous and then mixed with homogenizer and propeller mixing until completely uniform; added Phase B in a main vessel; to the main vessel, charged: glycerin, and mixed with a homogenizer and propeller for 10 minutes until completely uniform; adjusted the speed of the main vessel for mixing and slowly sifted: Pemulen TR-2 (B070) into the batch and then mixed for 45-60 minutes with propeller and recirculating homogenizer mixing; after completely uniform, stopped the homogenizer; continued propeller mixing slowly to mix out the foam before proceeding; charged SPD T-5 or CMF640TV slowly into the main batch and propeller mixed for at least 30 minutes; after the batch had been passed 2×through a microfluidizer, returned to batching vessel and mixed using propeller mixer; began cooling the vessel and adjusted the pH to between 7.20-8.00 utilizing the AMP Ultra PC 2000 and propeller mixed for at least 30 minutes until completely uniform; continued cooling the batch and mixed until cooled below 30° C.

A dispersion was prepared for use in the compositions of this disclosure and summarized below.

| | Dispersion Identification | A1 |
|---|---|---|
| Phase | Ingredient | Percentage |
| A | Water | 60.475 |
| C | Glycerin | 7.500 |
| E | Aminomethyl propanol | 0.275 |
| C | Benzyl Alcohol | 1.000 |
| E | Citric Acid, Water | Q.S. |
| D | C12-15 Alkyl Benzoate | 30.000 |
| B | Acrylates/C10-30 Alkyl | 0.750 |
| | Acrylate Crosspolymer | |
| | Total | 100.000 |

The dispersion A1 was prepared in accordance with the processes of this disclosure, in particular, measure D.I. water and added into a beaker; blended polymers together and added into water; mixed until fully hydrated; homogenized; added ingredients of Phase C into Phase AB; mixed well; added Phase D into Phase ABC; mixed for 20 min. and homogenized well; took 2 passes through a microfluidizer; adjusted pH in a range 5.20-5.35.

A dispersion was prepared for use in the compositions of this disclosure and summarized below.

| | Dispersion Identification | A2 |
|---|---|---|
| Phase | Ingredient | Percentage |
| A | Water | 56.40 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | Citric Acid, Water | 1.00 |
| E | Sodium Hydroxide, Water | 0.30 |
| B | Caprylic/Capric Triglyceride | 30.00 |
| D | Acrylates/C10-30 Alkyl | 0.80 |
| | Acrylate Crosspolymer | |
| | Total | 100.000 |

The dispersion A2 was prepared in accordance with the processes of this disclosure, in particular, combined Phase A and Phase C into a main vessel with propeller mixing; switched a main vessel to homogenizer and added Phase D slowly; added Phase B; passed through a microfluidizer twice with cooling; switched main vessel to homogenizer and added Phase D; switched main vessel to propeller mixer and adjusted pH with Phase E; adjusted to pH 6.00-6.50.

A dispersion was prepared for use in the compositions of this disclosure and summarized below.

| | Dispersion Identification | A3 |
|---|---|---|
| Phase | Ingredient | Percentage |
| A | Water | 56.40 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| C | Citric Acid, Water | 1.00 |
| E | Sodium Hydroxide, Water | 0.30 |
| B | Caprylic/Capric Triglyceride | 30.00 |
| D | Acrylates/C10-30 Alkyl | 0.80 |
| | Acrylate Crosspolymer | |
| | Total | 100.00 |

The dispersion A3 was prepared in accordance with the processes of this disclosure, in particular, combined Phase A and Phase C into a main vessel with propeller mixing; switched main vessel to homogenizer and added Phase D slowly; added Phase B; passed through a microfluidizer twice with cooling; switched main vessel to homogenizer and added Phase D; switched main vessel to propeller mixer and adjusted pH with Phase La Adjusted to pH 6.00-6.50.

A dispersion was prepared for use in the compositions of this disclosure and summarized below.

| | Dispersion Identification | A4 |
|---|---|---|
| Phase | Ingredient | Percentage |
| A | Water | 56.40 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| B | Dimethicone | 30.00 |
| C | Citric Acid, Water | 1.00 |
| E | Sodium Hydroxide, Water | 0.30 |
| D | Acrylates/C10-30 Alkyl | 0.80 |
| | Acrylate Crosspolymer | |
| | Total | 100.00 |

The dispersion A4 was prepared in accordance with the processes of this disclosure, in particular, combined Phase A and Phase C into a main vessel with propeller mixing; switched main vessel to homogenizer and added Phase D slowly; added Phase B; passed through a microfluidizer twice with cooling; switched main vessel to homogenizer and added Phase F; switched main vessel to propeller mixer and adjusted pH with Phase E; adjusted to pH 6.00-6.50.

A dispersion was prepared for use in the compositions of this disclosure and summarized below.

| | Dispersion Identification | A5 |
|---|---|---|
| Phase | Ingredient | Percentage |
| A | Water | 56.40 |
| A | Glycerin | 10.00 |
| A | Phenoxyethanol, | 1.00 |
| | Ethylhexylglycerin | |
| A | Potassium Sorbate | 0.25 |
| A | Sodium Benzoate | 0.25 |
| B | Dimethicone | 30.00 |
| C | Citric Acid, Water | 1.00 |
| E | Sodium Hydroxide, Water | 0.30 |
| D | Acrylates/C10-30 Alkyl | 0.80 |
| | Acrylate Crosspolymer | |
| | Total | 100.00 |

The dispersion A5 was prepared in accordance with the processes of this disclosure, in particular, combined Phase A and Phase C into a main vessel with propeller mixing; switched main vessel to homogenizer and added Phase D slowly; added Phase B; passed through a microfluidizer twice with cooling; switched main vessel to homogenizer and added Phase D; switched main vessel to propeller mixer and adjusted pH with Phase E; adjusted to pH 6.00-6.50.

Compositions of this disclosure were prepared by a process that involved preparing a premix containing (i) one or more hydrophobic agent(s), (ii) one or more polymers or copolymers, or combinations thereof, (iii) an aqueous-solute fluid, and optionally (iv) one or more additive(s); subjecting the premix to low energy mixing to form a first dispersion; and subjecting the first dispersion to ultra-high energy mixing to form a second dispersion. FIG. 1 depicts an illustrative process flow diagram for preparing the compositions of this disclosure.

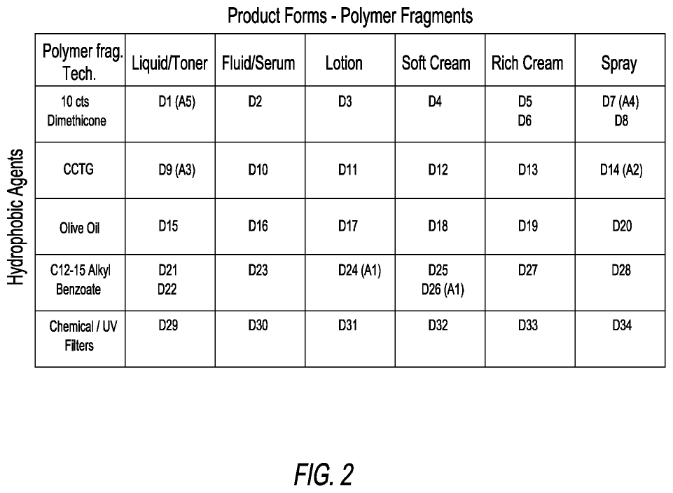

FIG. 2 shows the product type and hydrophobe for dispersion compositions of this disclosure, in accordance with the Examples.

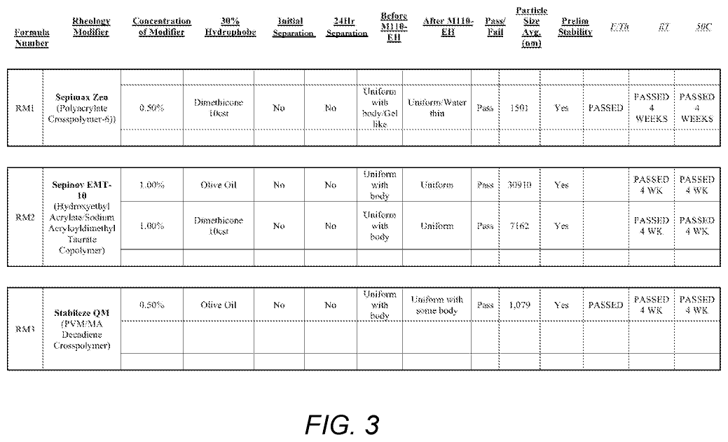

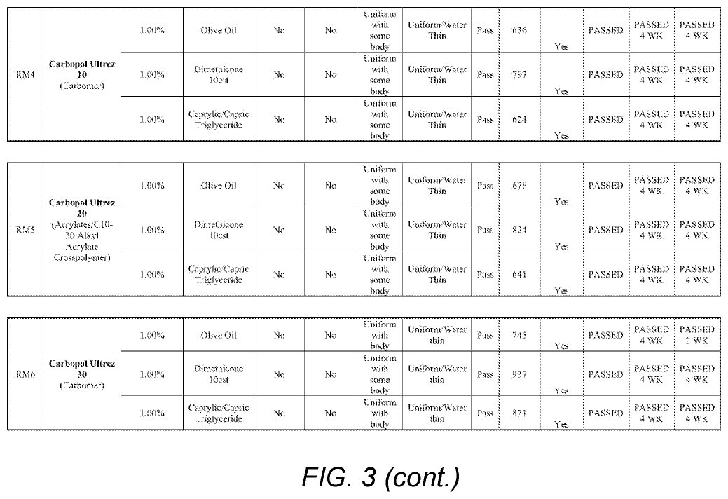

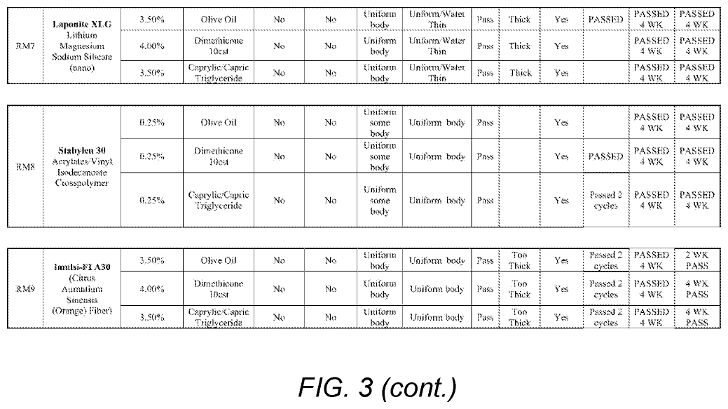

FIG. 3 shows dispersion assignment tracker data generated for compositions of this disclosure.

The data shows dispersion stability of the compositions of this disclosure.

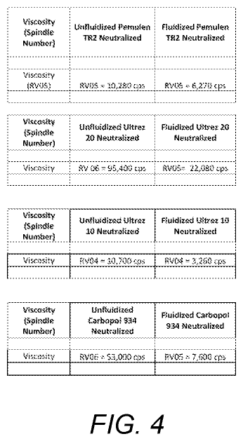

FIG. 4 shows viscosity versus shear data generated for compositions of this disclosure. The data shows that putting compositions containing the polymeric and copolymeric rheology modifiers of this disclosure through ultra-high energy mixing substantially decreases the viscosity of the polymeric and copolymeric rheology modifiers. As used in FIG. 4, Pemulen TR2 is a block copolymer of polyacrylic acid crosslinked with long-chained methacrylate, Ultrez 20 is acrylates/C10-30 alkyl acrylate crosspolymer, Ultrez 10 is carbomer or crosslinked acrylic acid polymers, and Carbopol 934 is crosslinked acrylic acid polymers.

What is claimed is:

1. A composition comprising:
a dispersion comprising (i) particles of one or more hydrophobic agent(s), (ii) one or more polymer or copolymer fragments, or combinations thereof, and (iii) an aqueous-solute fluid;
wherein the particles of one or more hydrophobic agent(s) are present in an amount from about 0.01% wt. to about 70% wt., the aqueous-solute fluid is present in an amount from about 1.0% wt. to about 98.5% wt., and the one or more polymer or copolymer fragments, or combinations thereof are present in an amount from about 0.01% wt. to about 10% wt., all based on the total weight of the composition; and
wherein the one or more polymer or copolymer fragments, or combinations thereof, are sufficient to stabilize the particles of one or more hydrophobic agent(s) in said dispersion, at a level from about 0.05% w/w to about 70% w/w of said one or more hydrophobic agent(s), in said dispersion.

2. The composition of claim 1, which is prepared by a process comprising:
preparing a premix comprising (i) one or more hydrophobic agent(s), (ii) one or more polymers or copolymers, or combinations thereof, (iii) an aqueous-solute fluid, and optionally (iv) one or more additive(s);
subjecting the premix to low energy mixing to form a first dispersion; and
subjecting the first dispersion to ultra-high energy mixing to form a second dispersion;
wherein the second dispersion comprises (i) particles of one or more hydrophobic agent(s), (ii) one or more polymer or copolymer fragments, or combinations thereof, and (iii) an aqueous-solute fluid;
wherein the particles of one or more hydrophobic agent(s) are present in an amount from about 0.01% wt. to about 70% wt., the aqueous-solute fluid is present in an amount from about 1.0% wt. to about 98.5% wt., and the one or more polymer or copolymer fragments, or combinations thereof are present in an amount from about 0.01% wt. to about 10% wt., all based on the total weight of the composition; and
wherein the one or more polymer or copolymer fragments, or combinations thereof, are sufficient to stabilize the particles of one or more hydrophobic agent(s) in said dispersion, at a level from about 0.05% w/w to about 70% w/w of said one or more hydrophobic agent(s), in said dispersion.

3. The composition of claim 1 wherein the particles of one or more hydrophobic agent(s) (i) have an average particle size from about 100 nm to about 5 m, (ii) have a solubility of less than about 0.1% by weight in water under standard conditions, and (iii) are 85% or more of a size within ±2.0 standard deviations, or within ±1.9 standard deviations, or within ±1.8 standard deviations, or within ±1.7 standard deviations, or within ±1.6 standard deviations, or within ±1.5 standard deviations, of the average particle size.

4. The composition of claim 2, wherein the one or more polymer or copolymer fragments, or combinations thereof, of the second dispersion exhibit at least a 10%, or 20%, or 30%, or 40%, or 50%, or 60%, or 70%, or 80%, or 90%, reduction in viscosity, as compared to the viscosity of the same one or more polymers, copolymers, or combinations thereof, of the first dispersion, under standard conditions.

5. The composition of claim 2, wherein the one or more polymer or copolymer fragments, or combinations thereof, of the second dispersion stabilize the particles of one or more hydrophobic agent(s) in said second dispersion for a greater period of time, when set to the same viscosity level, as compared to stabilization of the one or more hydrophobic agent(s) by the same one or more polymers, copolymers, or combinations thereof, of the first dispersion, under standard conditions.

6. The composition of claim 2 wherein the ultra-high energy mixing imparts a repulsive force that causes particles of the one or more hydrophobic agent(s) to repel or move away from each other in the second dispersion, thus enhancing the stability and dispersibility of the second dispersion.

7. The composition of claim 1 wherein the one or more polymer or copolymer fragments, or combinations thereof, provide a matrix that reduces Newtonian flow thereby reducing the number of hydrophobic particle collisions and potential coalescence.

8. The composition of claim 1, wherein the one or more polymer or copolymer fragments, or combinations thereof, are at least one selected from the group consisting of natural and synthetic polymer and copolymer fragments, and any derivatives or combinations thereof.

9. The composition of claim 8, wherein the one or more synthetic polymer or copolymer fragments, or combinations thereof, are at least one selected from the group consisting of fragments of the following: acrylates, methacrylates, acrylamides, vinyls, polyethylene and derivatives thereof, and any derivatives or combinations thereof.

10. The composition of claim 8, wherein the one or more synthetic polymer or copolymer fragments, or combinations thereof, are at least one selected from the group consisting of fragments of the following: carbomer and derivatives and salts thereof, acrylates/C₁₀-C₃₀ alkyl acrylate crosspolymer, acrylates/ceteth-20 itaconate copolymer, acrylates/ceteth-methacrylate copolymers, acrylates/steareth-20 methacrylate copolymers, acrylates/steareth-20 itaconate copolymers, acrylates/steareth-50 acrylate copolymers, acrylates/VA crosspolymers, acrylates/vinyl isodecanoate crosspolymers, acrylic acid/acrylonitrogen copolymers, ammonium acrylates/acrylonitrogen copolymers, glyceryl polymethacrylate, polyacrylic acid, PVM/MA decadiene crosspolymer, sodium acrylate/vinyl isodecanoate crosspolymers, sodium carbomer, ethylene/acrylic acid copolymer, ethylene/VA copolymer, acrylates/acrylamide copolymer, acrylate copolymers, acrylates/hydroxyester acrylate copolymers, acrylate/octylarylamide copolymers, acrylates/PVP copolymers, AMP/acrylate copolymers, butylester of PVM-MA copolymer, carboxylate vinyl acetate terpolymers, diglycol/CHDM/isophthalates/SIP copolymer, ethyl ester of PVM-MA copolymer, isopropyl ester of PVM-MA copolymer, octyl acrylamide/acrylate/butylaminoethyl methacrylate copolymers, polymethacrylamidopropyltrimonium chloride, polyvinyl caprolactam, PVP, PVP/dimethylaminoethylmethacrylate copolymer, PVP/DMAPA acrylate copolymers, PVP/carbamyl polyglycol ester, PVP/VA copolymer, PVP/VA vinyl propionate copolymer, PVP/vinylcaprolactam/DMAPA acrylate copolymers, sodium polyacrylate, VA/butyl maleate/isobornyl acrylate copolymers, VA/crotonates copolymer, VA/crotonates vinyl neodecanoate copolymer, VA/crotonates/vinyl propionate copolymer, vinyl caprolactam/PVP/dimethylaminoethylmethacrylate copolymer, hydroxyethyl acrylate/copolymer, sodium acrylate/sodium acryloyldimethyl taurate copolymer (and) C₁₅-C₁₉ alkane (and) polyglyceryl-6 laurate (and) polyglycerin-6, polyquarternium-37, ammonium acryloyldimethyltaurate/VP copolymer, hydroxyethyl acrylate/sodium acryloyldimethyl taurate copolymer, polyacrylate crosspolymer-6, and any derivatives or combinations thereof.

11. The composition of claim 8, wherein the one or more natural polymer or copolymer fragments, or combinations thereof, are at least one selected from the group consisting of fragments of the following: carbohydrate-based monomer units or derivatives thereof; proteins including casein; and any derivatives or combinations thereof.

12. The composition of claim 1, wherein the one or more hydrophobic agent(s) are at least one selected from the group consisting of: mono, di, tri, or poly alkyl (or alkenyl) esters, ethers, acetals and hemiacetals of a di-, tri-, or polyhydroxy compound, and any derivatives or combinations thereof; saturated and unsaturated, linear and branched natural or vegetable oils selected from the group consisting of soybean oil, almond oil, castor oil, canola oil, cottonseed oil, grapeseed oil, rice bran oil, palm oil, coconut oil, palm kernel oil, olive oil, linseed oil, sunflower oil, safflower oil, peanut oil, corn oil, and any derivatives or combinations thereof; water insoluble silicone materials selected from the group consisting of polyalkylsiloxanes, polyarylsiloxanes, polyalkylarylsiloxanes, polysiloxane gums, polyethersiloxane copolymers, silicone crosspolymers, and any derivatives or combinations thereof; water insoluble materials selected from the group consisting of diglycerides, triglycerides, lauramine oleate, isopropyl palmitate, mineral oil, petrolatum, and any derivatives or combinations thereof; and hemp oil and cannabinoids selected from the group consisting of cannabidiol (CBD), delta-9-tetrahydrocannabinol (THC), cannabinol (CBN), cannabigerol (CBG), cannabichromene oil (CBC), any hemp oil or cannabinoid species, and any derivatives or combinations thereof.

13. The composition of claim 1, wherein the one or more hydrophobic agent(s) comprise one or more therapeutic agent(s).

14. The composition of claim 2, further comprising: adding one or more functional agent(s) to the premix, or post-treating the first dispersion with one or more functional agent(s), or post-treating the second dispersion with one or more functional agent(s); wherein the one or more functional agent(s) are at least one selected from the group consisting of surfactants, neutralizing agents, chelating agents, foaming agents, rheological modifying agents, polypeptide rheological modifying agents, sensates, and any derivatives or combinations thereof.

15. The composition of claim 13, wherein the one or more therapeutic agent(s) comprise one or more hydrophobic agent(s) or one or more hydrophilic agent(s), and are at least one selected from the group consisting of: anti-acne agents, antimicrobial agents, anti-inflammatory agents, analgesics, anti-erythemal agents, anti-pruritic agents, anti-edemal agents, anti-psoriatic agents, anti-fungal agents, skin protectants, sunscreen agents, vitamins, antioxidants, scavengers, anti-irritants, anti-bacterial agents, antiviral agents, antiaging agents, photoprotection agents, hair growth enhancers, hair growth inhibitors, hair removal agents, antidandruff agents, anti-seborrheic agents, exfoliating agents, wound healing agents, anti-ectoparasitic agents, sebum modulators, immunomodulators, hormones, botanicals, moisturizing agents, hand sanitizing agents, astringents, sensates, antibiotics, anesthetics, steroids, tissue healing substances, tissue regenerating substances, amino acids, peptides, minerals, ceramides, biohyaluronic acids, bleaching ingredients, hemp oils, cannabinoids, and any derivatives or combinations thereof.

16. The composition of claim 1, wherein the one or more hydrophobic agent(s) comprise at least one aesthetic modifying agent.

17. The composition of claim 2, further comprising treating or post-treating the premix, or post-treating the first dispersion, or post-treating the second dispersion, such that: pH is raised or lowered by the addition of an alkali or acid, respectively; viscosity is increased or decreased by the addition of a thickening agent or salt, respectively; specific gravity is adjusted by the addition of one or more of an antifoam, centrifugation, vacuum, and reduction in viscosity with sweeping mixing; refractive index is adjusted up or down by the addition of a high refractive index solvent/solute or water; or active levels are adjusted by the addition of a hydrophobic active dispersion.

18. The composition of claim 16, wherein the one or more hydrophobic aesthetic modifying agent(s) comprise at least one compound selected from the group consisting of: polysiloxanes, cyclic siloxanes, polyalkylsiloxanes, polyarylsiloxanes, polyalkylarylsiloxanes, polysiloxane gums, polyethersiloxane copolymers, and silicone crosspolymers.

19. The composition of claim 1, wherein the aqueous-solute fluid comprises water, or a combination of water and one or more polar solute(s); wherein the one or more polar solutes have a water solubility of 0.1% or greater at a temperature of 23° C. and a pressure of 100 kPa (1 bar), and have a dielectric constant of greater than 10 at a temperature of 23° C. and a pressure of 100 kPa (1 bar).

20. The composition of claim 19, wherein the one or more polar solutes are selected from the group consisting of: water soluble solids at a temperature of 23° C. and a pressure of 100 kPa (1 bar), water soluble liquids at a temperature of 23° C. and a pressure of 100 kPa (1 bar), and any derivatives or combinations thereof.

21. The composition of claim 20, wherein the water soluble solids are at least one selected from the group consisting of: carbohydrates; amino acids, peptides, and proteins; vitamins; minerals; and any derivatives or combinations thereof; and wherein the water soluble liquids comprise flowable, non-viscous, semi-viscous, or viscous liquids.

22. The composition of claim 20, wherein the water soluble liquids are at least one selected from the group consisting of: glyceraldehyde, erythrose, erythrulose, sedoheptulose, and any derivatives or combinations thereof.

23. The composition of claim 20, wherein the water soluble liquids comprise water miscible liquids; wherein the water miscible liquids are at least one selected from the group consisting of: acetaldehyde, acetic acid, acetone, 1,2-butylene glycol, 1,3-butylene glycol, 1,4-butylene glycol, 2-butoxyethanol, dimethyl sulfoxide, ethanol, ethoxydiglycol, triethylene glycol, ethylene glycol, methanol, isopropanol, 1,2-propylene glycol, 1,3-propylene glycol, 1-propanol, propanoic acid, diglycerin, polyglycerol, glycerin, 1.5-pentylene glycol, hexylene glycol, and any derivatives or combinations thereof.

24. The composition of claim 2, wherein the one or more additive(s) comprise one or more active or therapeutic ingredient(s); wherein the one or more active or therapeutic ingredient(s) are at least one selected from the group consisting of: anti-acne agents, antimicrobial agents, anti-inflammatory agents, analgesics, anti-erythemal agents, antipruritic agents, antiedemal agents, anti-psoriatic agents, antifungal agents, skin protectants, sunscreen agents, vitamins, antioxidants, anti-irritants, anti-bacterial agents, antiviral agents, antiaging agents, photoprotection agents, exfoliating agents, wound healing agents, sebum modulators, immunomodulators, hormones, botanicals, moisturizing agents, hand sanitizing agents, astringents, sensates, antibiotics, anesthetics, steroids, tissue healing substances, tissue regenerating substances, amino acids, peptides, minerals, ceramides, hyaluronic acids, skin bleaching ingredients, pre-biotics, probiotics, hemp oils, cannabinoids, and humectants.

25. The composition of claim 1 for use in one or more of nutritional, pharmaceutical, biomedical, cosmetic, food, animal care, household, veterinary health, and pet care applications.

26. The composition of claim 1, which is in the form of, or formulated as, a light liquid, heavy liquid, gel, light or soft cream, heavy or rich cream, light lotion, viscous lotion, butter, balm, stick, spray, mist, light fluid, rich fluid, liquid/toner, fluid/serum, or foam.

27. The composition of claim 1, which is formulated as a sunscreen lotion, a sunscreen spray, a sunscreen toner, a moisturizing hand sanitizer, a body lotion, a body spray, a natural oil liquid, a natural oil spray, a natural oil toner, an exfoliating mask, a lubricating shaving gel, a cream, a hemp oil or cannabinoid or any variants, hybrids, or derivatives thereof.

28. A process comprising:
preparing a premix comprising (i) one or more hydrophobic agent(s), (ii) one or more polymers or copolymers, or combinations thereof, (iii) an aqueous-solute fluid, and optionally (iv) one or more additive(s);
subjecting the premix to low energy mixing to form a first dispersion; and
subjecting the first dispersion to ultra-high energy mixing to form a second dispersion;
wherein said second dispersion comprises (i) particles of one or more hydrophobic agent(s) having an average particle size of about 5 m or less, (ii) one or more polymer or copolymer fragments, or combinations thereof, and (iii) an aqueous-solute fluid; and
wherein the one or more polymer or copolymer fragments, or combinations thereof, are present in an amount sufficient to stabilize the particles of one or more hydrophobic agent(s) in said second dispersion.
